(19) United States
(12) Patent Application Publication
(10) Pub. No.: US 2026/0042836 A1
(43) Pub. Date: Feb. 12, 2026

(54) BTN2A1 BINDING PEPTIDE

(71) Applicant: UMC Utrecht Holding B.V., Utrecht (NL)

(72) Inventors: Jürgen Herbert Ernst Kuball, Utrecht (NL); Dennis Beringer, Utrecht (NL); Eline Van Diest, Utrecht (NL)

(21) Appl. No.: 19/140,075

(22) PCT Filed: Dec. 19, 2023
(86) PCT No.: PCT/EP2023/086699

§ 371 Date: Jun. 17, 2025

(30) Foreign Application Priority Data
Dec. 19, 2022 (EP) 22214633.4

(51) Int. Cl. C07K 16/28

(52) U.S. Cl. CPC C07K 16/2803; C07K 16/2809; C07K 2317/31; C07K 2317/92

(57) ABSTRACT

Disclosed is a Butyrophilin Subfamily 2 Member A1 (BTN2A1) binding peptide with an amino acid sequence comprising an amino acid other than glutamic acid, for example, tryptophan, at a position corresponding to position 22 shown in SEQ ID NO: 1; an amino acid other than arginine, for example, tyrosine, at a position corresponding to position 73 shown in SEQ ID NO: 1; and/or an amino acid other than threonine, for example, histidine or tryptophan, at a position corresponding to position 81 shown in SEQ ID NO: 1. The BTN2A1 binding peptide may be comprised in an (exogeneous) immune receptor or extracellular domain thereof, preferably a γδ T-cell receptor or extracellular domain thereof, more preferably a γ9δ2 T-cell receptor or extracellular domain thereof. This disclosure particularly relates to use in therapy, preferably for use in treatment of a cancer and/or an infection, or for use in diagnostic methods.

### CROSS-REFERENCE TO RELATED APPLICATIONS

This application is a national phase entry under 35 U.S.C. § 371 of International Patent Application PCT/EP2023/086699, filed Dec. 19, 2023, designating the United States of America and published as International Patent Publication WO 2024/133301 A1 on Jun. 27, 2024, which claims the benefit under Article 8 of the Patent Cooperation Treaty of European Patent Application Serial No. 22214633.4, filed Dec. 19, 2022.

### STATEMENT REGARDING ELECTRONIC FILING OF A SEQUENCE LISTING

Pursuant to 37 C.F.R. § 1.831 through 1.835, a Sequence Listing XML file entitled “6Z50731-eolf-seql,” 68,351 bytes in size, generated May 13, 2025, has been submitted via EFS-Web is provided in lieu of a paper copy. This Sequence Listing is hereby incorporated by reference into the specification for its disclosures.

### TECHNICAL FIELD

This disclosure relates to BTN2A1 binding peptides, in particular, Vgamma9 TCR domains, and more, in particular Vγ9Vδ2 TCR based therapeutics, which can be used in treatment of cancer, autoimmune diseases or in treatment of infections.

### BACKGROUND

Targeting tumors with immune cells have gained great momentum over the past decades and many different immune cell subsets have been explored for immunotherapy. One of the most successful immune subset for translation have been the T cells. Many different strategies have been developed to harness the power of T cells, such as immune checkpoint blockade, bispecific T cell engagers, and genetically engineered T cells, i.e., CAR T cells [1, 2]. Many of these strategies were mainly taking the major T cell lineage, αβT cells, into account.

However, a smaller T cell lineage, the γδT cells, are currently attracting a lot of attention because of their unique features. Located on the cusp of the innate and adaptive systems they combine features of both, they have an innate-like responsiveness but are also able to form immunological memory. The γδT cell linage can be divided in two subsets, the Vγ9Vδ2 T cells and the Vδ2-negative γδT cells. The last subset has been linked to more adaptive features [3], as a result T cells belonging to the Vδ2-negative T cell subset have been shown to recognize a plethora of antigens in a clonotypic manner [4]. On the other hand, the Vγ9Vδ2 T cells are classified as invariant T cells, all using the same TCR V-genes and recognizing the same antigen complex [5], akin to iNKT cells recognizing CD1d [6] or MAIT cells recognizing MR1 [7]. A commonality of both γδT cell is that they can recognize and kill a broad spectrum of tumors, which led to many different strategies to use these γδT cells and their γδT cell receptors (TCR) as immunotherapy [8].

T cells bearing a Vγ9Vδ2 TCR can recognize intracellular phosphoantigens that originate either from microbial pathogens or from a dysregulated mevalonate pathway in the case of stressed or malignant cells [9]. These phosphoantigens bind to the intracellular domain B30.2 of BTN3A1 [10, 11] resulting in the formation of a complex between the intracellular domains of BTN3A1 and BTN2A1 [12]. Recent studies have reported that the Vγ9 domain of the Vγ9Vδ2 TCR interacts with BTN2A1 using germline encoded residues, independent of the CDR3γ residues, and that this interaction is essential for the activation of Vγ9Vδ2 T cells [13, 14]. Both studies determined the affinity of this interaction to be 40-50 μM, which is a typical TCR-ligand affinity [15], but the interaction with BTN2A1 alone may not be sufficient for T cell activation. The previously identified phosphoantigen sensing molecule BTN3A1 was, next to BTN2A1, still important for Vγ9Vδ2T cell activation [13, 14, 16]. Although the interaction between the extracellular domains of BTN3A1 and Vγ9Vδ2 TCRs has not been formally shown, it was recently reported that a fusion protein consisting of the extracellular domains of BTN2A1 and BTN3A1 was able to activate Vγ9Vδ2 T cells in presence of a costimulatory signal [17].

Vγ9Vδ2 TCRs have varying potencies when used as Vγ9Vδ2 TCR-based therapeutic or diagnostic agents [16, 18-20]. This potency is linked to the binding strength (affinity) of Vγ9Vδ2 TCRs to tumor cells [16], and ‘strong’ binding Vγ9Vδ2 TCRs lead to better and higher functional avidity, as indicated by and augmented tumor control in vitro and in xenograft mice models [19, 20]. Vγ9Vδ2 TCRs frequently are not potent enough to induce (lasting) tumor control in most pre-clinical in vivo models.

Several therapeutic strategies have been developed that use the tumor recognition capacity of the Vγ9Vδ2 TCR. One of these strategies, the TEG concept [19, 21, 22], is currently tested in a phase I clinical trial, while γδTCR-antibody fusion protein [23] and γδTCR-antiCD3 bispecific molecules [20] are in preclinical testing. For many therapeutic strategies that use TCRs, the receptor-ligand interaction is enhanced to, in general, sub-micromolar affinity to enhance tumor clearance in vivo [24-26]. As the tumor reactivity is directly linked to the binding strength of Vγ9Vδ2 TCRs to its ligand-complex, affinity maturation of Vγ9Vδ2 TCRs was considered to increase the potency of Vγ9Vδ2 TCR-based therapeutics mentioned above. However, approaches for affinity enhancement of Vγ9Vδ2 TCRs using, e.g., phage display, turned out to be a dead end due to the poor expression of these TCRs or variable TCR fragments e.g., in E. coli [27].

Therefore, there remains a need to develop new approaches for affinity enhancement of Vγ9Vδ2 and/or to identify Vγ9Vδ2 TCR variants with increased ligand-complex affinity, in particular, to identify Vγ9 domain variants with increased binding affinity to BTN2A1.

### BRIEF SUMMARY

It was considered that an alternative strategy for affinity maturation of the Vγ9 domain for the binding to BTN2A1 was needed to create more potent Vγ9Vδ2 TCR based therapeutics. Previous attempts to affinity maturate the Vγ9Vδ2TCR, e.g., by using phage display, were unsuccessful due to, for example, low expression in E. coli.

An efficient screening method was developed based on the recently developed Gamma delta TCR anti-CD3 bispecific molecules (GABs) [20] to identify more potent Vγ9Vδ2 TCR mutants for immunotherapy and characterize Vγ9Vδ2 TCR mutants that mediate an improved functional avidity as well as increased stability. This disclosure can be used to create more potent diagnostic and therapeutic agents if based on Vγ9-chain, in particular, the BTN2A1 binding peptide thereof.

In particular, a mutant BTN2A1 binding amino acid sequence (i.e., peptide) in the Vγ9 domain was identified, which can increase the affinity for BTN2A1 by -10 fold, and which allows for more potent T cell activation and killing of tumor cells, for example, through GABs or TEGs (TEG=T cell Engineered to express a defined GdTCR). It was found that modification(s) Vγ9_{E22W}, Vγ9_{T91H}, and/or Vγ9_{T81W} enhance(s) affinity of the peptide for BTN2A1 and can, for example, be used to enhance activity of GABs or other g9d2TCR based immune therapies or when the g9d2TCR is used as diagnostic tool.

Accordingly, this disclosure relates to a (combination of) mutation(s) in the BTN2A1 binding peptide in the Vgamma9 chain of a gamma-delta T cell receptor (gdTCR) that enhances the potency of gdTCR based therapeutic agents, like alpha-beta T cells engineered to express a defined gdTCR (TEGs) and gdTCR anti-CD3 bispecific molecules (GABs), and/or increase the expression levels of recombinant Vgamma9 containing gdTCRs, in the case of GABs.

The Vgamma9 chain is able to pair with all Vdelta (Vdelta1 . . . . Vdelta8) chains to form a gdTCR, but most commonly the Vgamma9 chain pairs with a Vdelta2 chain, resulting in the Vγ9Vδ2TCR. This combination of gamma and delta TCR chains senses intracellular phosphoantigen levels in cells, which can be elevated when the metabolism of the cell is disturbed, e.g., in the case of an infection or when a cell undergoes malignant transformation. An increase in intracellular phosphoantigen levels leads to the relocation and complexation of the butyrophilin molecules BTN2A1 and BTN3Al-BTN3A2/A3-heterodimers, which triggers Vγ9Vδ2TCR mediated T cell activation. Foreseen is a potent anti-cancer drug, but this disclosure may also find use as a therapeutic agent to clear infected cells, or use as a diagnostic tool, i.e., for determining cellular BTN2A1 expression.

Furthermore, a mutant BTN2A1 binding peptide was identified that increases its stability, e.g., in GABs. It was found that modification Vγ9_{R73Y} enhances stability of the BTN2A1 binding peptide in the Vγ9 chain, which in turn allows for enhanced stability of GABs. This modification can increase cellular expression and thus production yield per volume of culture, which currently is an important obstacle in the production of GABs, making the production relatively costly. The mutation increases the production yield at least 2 fold, and is also useful for stabilizing β chains.

In addition, a mutant BTN3A1/A2/A3 binding peptide was identified, which provides for enhanced binding affinity to BTN3A1/A2/A3, and/or which allows for more potent T cell activation and killing of tumor cells, for example, through GABs or TEGs. The BTN3A1/A2/A3 binding peptide according to this disclosure and the BTN2A1 binding peptide according to this disclosure may be combined, such as in a γδ T-cell receptor or extracellular domain thereof, more preferably a γ9δ2 T-cell receptor or extracellular domain thereof, wherein preferably the BTN2A1 binding peptide is a T-cell receptor γ-chain domain, preferably a T-cell receptor γ9-chain domain and/or the BTN3A1/A2/A3 binding peptide is a T-cell receptor δ-chain domain, preferably a T-cell receptor δ2-chain domain.

### BRIEF DESCRIPTION OF THE DRAWINGS

FIG. 1 analysis of the Vγ9-BTN2A1 interface for predicting affinity enhancing mutations. (A) the Vγ9-BTN2A1 interface as predicted by Haddock. BTN2A1 is in cartoon representation and the Vγ9 TCR is in surface representation. Vγ9-residues that are at the binding interface with BTN2A1 are indicated in dark gray. (B) Cartoon representation of Vγ9Vδ2TCR (pdb 1hxm) where the residues predicted to be involved in the binding with BTN2A1 are shown in dark gray and their sidechains represented as sticks. Both figure A and B were generated using PyMol. (C) Amino acid sequence of the Vγ9 domain, CDR residues are in italic font, and interface residues are indicated in black. (D) Limited overlap in the Top25 predicted mutations by the four different software packages; EvoEF, Elaspic2 (ddG and EL2 score), and mCSM-PPI2. Venn diagram was generated using R studio.

FIG. 2 Vγ9_{R73Y} mutation increases the production of GABs without affecting activity. (A) GAB expression levels in HEK293F culture media were determined by western blot 6 days after transfection. The C-terminal poly-His tag of the GAB was used for detection with a 6×His antibody. Western blots of 2 separate transfections are shown. (B) GAB yield after purification of GABs without and with the Vγ9_{R73Y} mutation. Significance was calculated using a Welch's t test in GraphPad Prism (v8.3.0). (C) IFNγ release by T cells after overnight co-culture with RPMI8226 cells in presence of GABs and 30 μM pamidonate. IFNγ concentration in the co-culture media was determined by ELISA, plotted as mean+SD (n=2). (D) GAB expression levels in HEK293F culture media were determined by western blot 6 days after transfection for additional mutations at position Vγ9_{R73}, as in A. (E) Relative quantification of GAB expression levels compared to GAB-AJ8-Vγ9_{R73Y} (n=2).

FIG. 3 Buried arginine in Vγ9 is also present in half of the V3 genes. (A) Sequence probability plot of all functional V3 genes of IMGT residue numbers 75-84. Probability plot was generated using WebLogo 3.7.4 (B) Buried arginine “R73” in TRGV9 (pdb 1hxm) and (C) the equivalent arginine in TRBV6 (pdb 2bnu). Figures were generated using PyMol.

FIG. 4 the effect of single Vγ9 mutations on GAB expression levels and GAB activity. (A) GAB expression levels in HEK293F media were analyzed by dot-blot using an anti-6×His antibody. The results are shown as relative intensity of the dot-blot signal compared to the GAB-AJ8s dot-blot signal. Each dot represents the GAB expression level of a HEK293F transfection relative to GAB-AJ8s on the same dot-blot. (B) Relative IFNγ secretion induced by the GAB-AJ8s-Vγ9 mutants compared to GAB-AJ8s. IFNγ was measured after a co-culture of T cells and tumor cells, either MZ1851RC or SCC9, in presence of GAB expression media and 30 M pamidronate. Dots represent individual measurements normalized to the corresponding GAB-AJ8s condition in the same assay.

FIG. 5 Vγ9_{E22W} mutation enhances the activity of purified GAB substantially. IFNγ release was measured after a 24 h co-culture of T cells and tumor cells using ELISA. T cells were co-cultured with three different tumor cell lines in a 1:1 E:T ratio in presence of GAB, at different concentrations, and 30 M pamidronate.

FIG. 6 Vγ9_{E22W} mutation in GAB-AJ8s induced more tumor cell killing compared to GAB-AJ8s. (A-C) T cells and indicated luciferase transduced target cells were co-cultured for 20 h in the presence of GAB and 30 M pamidronate at a 10:1 effector-target ratio. The fraction of living cells was quantified by measuring luciferase activity, signals were normalized to T cells with target cells without GAB. Mean (+S.D.) are plotted n=3. (D) For each GAB titration EC50 values were calculated in GraphPad Prism 9.3.0. The three EC50 values for GAB-AJ8s and GAB-AJ8sγE22W were plotted and significance was calculated using a ratio t-test in GraphPad Prism 9.3.0.

FIG. 7 The Vγ9_{E22W} mutation enhances the activity of GAB with different CDR3δ sequences substantially. IFNγ release was measured after a 24 h co-culture of T cells and tumor cells using ELISA. T cells were co-cultured with two different tumor cell lines in a 1:1 E:T ratio in presence of GAB, at different concentrations, and without or with 10 or 30 M pamidronate. GAB dilution series for: (A) GAB-AJ8s, which has a CDR3δ resulting in intermediate activity, (B) GAB-A3s, which has a CDR3δ resulting in strong activity, and (C) GAB-LM1s, which has a CDR3δ resulting in very low activity. Dots represent mean of two biological replicates, error bars represent standard deviation.

FIG. 8 The Vγ9_{E22W} mutation enhances the activity of αβT cells transduced with a functional γ9δ2TCR significantly. Killing of RMPI8226-lucGFP cells by αβT cells transduced with 4 different 7962TCR after 20 hours of co-culture. Dots represent mean of 2 replicates at different E:T ratio (10:1, 3:1, and 1:1) and with or without 30 μM pamidronate. Significance was calculated using a one-way ANOVA in GraphPad Prism (v8.3.0), * p<0.05, ** p<0.01, ***p<0.005.

FIG. 9 the effect of single Vγ9 mutations in important residues on GAB activity. (A) Based on the reactivity of the first set of Vγ9 mutations residues were dived in different classes. Important residues cluster together on the Vγ9 domain. (B) amino acid sequence of the Vγ9 domain, CDR residues are in italic font, Vγ9R73 is underlined, Vγ9E22 is in dark gray and bold, important residues are in dark gray., and permissive residues/non tested are in mid-gray. (C) Relative IFNγ secretion induced by the GAB-AJ8s-Vγ9 mutants compared to GAB-AJ8s. IFNγ was measured after a co-culture of T cells and tumor cells, MZ1851RC, in presence of GAB expression media and 30 M pamidronate. Dots represent individual measurements normalized to the corresponding GAB-AJ8s condition in the same assay. (D) Top 5 of Vγ9 mutations, which induced the highest relative IFNγ release.

FIG. 10 The Vγ9_{T91H} and Vγ9_{T81W} mutations in GAB-AJ8s induced better T cell response compared to GAB-AJ8s. (A) IFNγ release was measured after a 24 h co-culture of T cells and tumor cells using ELISA. T cells were co-cultured with three different tumor cell lines in a 1:1 E:T ratio in presence of GAB, at different concentrations, and 30 M pamidronate. (B) T cells and luciferase transduced target cells were co-cultured for 20 h in the presence of GAB and 30 μM pamidronate at a 5:1 effector-target ratio. The fraction of living cells was quantified by measuring luciferase activity, signals were normalized to T cells with target cells without GAB. Mean (+S.D.) are plotted n=2. (C) For each GAB titration EC50 values were calculated in GraphPad Prism 9.3.0. The EC50 values of GAB-AJ8s_{γT81H}, GAB-AJ8s_{γT81W} and GAB-AJ8s_{γT83I} mutations were compared to the EC50 values of GAB-AJ8s for significance, using a ratio t-test in GraphPad Prism 9.3.0.

FIG. 11 the effect of single Vγ9-E22 mutations on GAB activity. (A) IFNγ was measured after a co-culture of T cells and tumor cells, MZ1851RC, in presence of GAB expression media of indicated GAB-AJ8s-mutant and 30 μM pamidronate. Dots represent measurements of unique co-cultures. (B) IFNγ release was measured after a 24 h co-culture of T cells and tumor cells using ELISA. T cells were co-cultured with either RPMI8226 or SCC9 in a 1:1 E:T ratio in presence of indicated purified GAB, at different concentrations, and 30 M pamidronate.

FIG. 12 Vδ2 mutations K53S and K53S enhance the activity of GAB-AJ8s. (A) IFNγ release was measured after a 24 h co-culture of T cells and tumor cells using ELISA. T cells were co-cultured with RPMI8226-lucGFP in a 1:1 E:T ratio in presence of indicated purified GAB, at different concentrations, and 30 μM pamidronate. (B) T cells and luciferase transduced RPMI8226 tumor cells were co-cultured for 20 h in the presence of GAB and 10 M pamidronate at a 5:1 effector-target ratio. The fraction of living cells was quantified by measuring luciferase activity, signals were normalized to T cells with target cells without GAB.

FIG. 13 hydrophobic Vδ2-G31 mutations enhance GAB activity. (A) IFNγ was measured after a co-culture of T cells and tumor cells, MZ1851RC, in presence of GAB expression media of indicated GAB-AJ8s_{δG31}-mutants and 30 M pamidronate. Dots represent measurements of unique co-cultures. (B) IFNγ release was measured after a 24 h co-culture of T cells and tumor cells using ELISA. T cells were co-cultured with either RPMI8226 or SCC9 in a 1:1 E:T ratio in presence of indicated purified GAB, at different concentrations, and 30 M pamidronate.

FIG. 14 Combining Vγ9_{E22W} with Vδ2_{G31V} or Vδ2_{K53S} in GAB-AJ8s leads to superior GAB activity (A) IFNγ release was measured after a 24 h co-culture of T cells and tumor cells using ELISA. T cells were co-cultured with either RPMI8226-lucGFP or SCC9-lucGFP in a 1:1 E:T ratio in presence of indicated purified GAB, at different concentrations, and 30 M pamidronate. (B) T cells and luciferase transduced RPMI8226 or SCC9 tumor cells were co-cultured for 20 h in the presence of GAB and 10 μM pamidronate at a 5:1 effector-target ratio. The fraction of living cells was quantified by measuring luciferase activity, signals were normalized to T cells with target cells without GAB.

### DETAILED DESCRIPTION

In a first aspect, this disclosure relates to a Butyrophilin Subfamily 2 Member A1 (BTN2A1) binding peptide.

BTN2A1 is a butyrophilin and member of the butyrophilin family of transmembrane (TM) proteins of which 8 members (BTN1A1, BTN2A1/2A2, BTN3A1/3A2/3A3, MOG, and BTNL2) are located in the major histocompatibility complex (MHC) class I region of human chromosome 6.

BTN2A1 binding is important for recognizing intracellular phosphoantigens that originate either from microbial pathogens or from a dysregulated mevalonate pathway in the case of stressed or malignant cells [9], e.g., as occurring in cancer or infectious disease. These phosphoantigens bind to the intracellular domain B30.2 of BTN3A1 [10, 11] resulting in the formation of a complex between the intracellular domains of BTN3A1 and BTN2A1 [12], leading to a complex of BTN2A1-homodimer with BTN3A1-BTN3A2/A3 heterodimer [77]. Recent studies have reported that the BTN2A1 binding peptide of the Vγ9 domain of the Vγ9Vδ2 TCR interacts with BTN2A1 using germline encoded residues, independent of the CDR3y residues, and that this interaction is important for the activation of Vγ9Vδ2 T cells [13, 14]. The BTN2A1 binding peptide according to this disclosure may combined with a BTN3A1 binding moiety, e.g., a delta (2) chain, for example, together in a γδ TCR or extracellular domain thereof (e.g., Vγ9Vδ2 TCR or extracellular domain thereof).

The BTN2A1 binding peptide of this disclosure comprises an amino acid sequence that binds BTN2A1, wherein the amino acid sequence preferably has at least 50, 60, 70, 80, 90, 91, 92, 93, 94, 95, 96, 97, 98, 99, 100% sequence identity with SEQ ID NO: 1, wherein the amino acid sequence comprises an amino acid other than glutamic acid at a position corresponding to position 22 as shown in SEQ ID NO: 1.

In addition or alternatively, the BTN2A1 binding peptide may comprise an amino acid other than arginine at a position corresponding to position 73 as shown in SEQ ID NO: 1.

In addition or alternatively, the BTN2A1 binding peptide may comprise an amino acid other than threonine at a position corresponding to position 81 as shown in SEQ ID NO: 1.

In addition or alternatively, the BTN2A1 binding peptide of this disclosure comprises an amino acid sequence that binds BTN2A1, wherein the amino acid sequence preferably has at least 50, 60, 70, 80, 90, 91, 92, 93, 94, 95, 96, 97, 98, 99, 100% sequence identity with SEQ ID NO: 43, wherein the amino acid sequence comprises an amino acid other than glutamic acid at a position corresponding to position 22 as shown in SEQ ID NO: 43.

In addition or alternatively, the BTN2A1 binding peptide may comprise an amino acid other than arginine at a position corresponding to position 73 as shown in SEQ ID NO: 43.

In addition or alternatively, the BTN2A1 binding peptide may comprise an amino acid other than threonine at a position corresponding to position 81 as shown in SEQ ID NO: 43.

It was found that mutating the amino acid at position 22 and/or 81 as naturally occurring in the BTN2A1 binding peptide in human TCRy9 chain variable domain (SEQ ID NO:1 or 43) surprisingly increases affinity of the peptide for BTN2A1, in case of mutating at position 22 the affinity for BTN2A1 is increased more than 10-fold. The term affinity refers to the strength of a binding reaction between a binding domain and an epitope, e.g., the peptide and BTN2A1. It is the sum of the attractive and repulsive forces operating between the binding domain and the epitope. The term affinity, as used herein, refers to the apparent binding affinity, which is determined as the equilibrium dissociation constant (Kd). A high affinity binding domain preferably has a binding affinity Kd for its specific epitope of less than 10⁻⁸ M, preferably less than 10⁻⁹ M. A low affinity binding domain preferably has a binding affinity Kd for its specific epitope of more than 10⁻⁸ M, preferably more than 10⁻⁷ M.

In addition, it was found that mutating the amino acid at position 73 as naturally occurring in the BTN2A1 binding peptide in human TCRy9 chain variable domain (SEQ ID NO:1 or 43) surprisingly increases stability of the BTN2A1 binding peptide, and can increase production yield (e.g., in grams of BTN2A1 binding peptide or construct comprising said) per volume of culture by more than 2-fold.

This disclosure particularly relates to the BTN2A1 binding peptide as described herein, wherein the amino acid other than glutamic acid at a position corresponding to position 22 is tryptophan (or conservative substitution thereof), for example, the amino acid other than glutamic acid at a position corresponding to position 22 is a tryptophan or phenylalanine, most preferably the amino acid other than glutamic acid at a position corresponding to position 22 is tryptophan.

In addition or alternatively, the amino acid other than arginine at a position corresponding to position 73 is tyrosine (or conservative substitution thereof), for example, the amino acid other than arginine at a position corresponding to position 73 is tyrosine, tryptophan or phenylalanine.

In addition or alternatively, the amino acid other than arginine at a position corresponding to position 81 is histidine (or conservative substitution thereof), or tryptophan (or conservative substitution thereof), for example, the amino acid other than arginine at a position corresponding to position 81 is histidine, arginine, lysine, tryptophan, phenylalanine or tyrosine.

In addition or alternatively, the BTN2A1 binding peptide according to this disclosure comprises:
a threonine (or conservative substitution thereof), for example, a threonine, alanine, serine or glycine at a position corresponding to position 18 as shown in SEQ ID NO: 1 (or 43);
an arginine (or conservative substitution thereof), for example, an arginine, histidine, or lysine at a position corresponding to position 20 as shown in SEQ ID NO: 1 (or 43);
a glutamic acid (or conservative substitution thereof), or leucine (or conservative substitution thereof), for example, a glutamic acid, aspartic acid, asparagine, and glutamine, leucine, methionine, isoleucine, valine or cysteine at a position corresponding to position 70 as shown in SEQ ID NO: 1 (or 43);
an aspartic acid (or conservative substitution thereof), for example, an aspartic acid, glutamic acid, asparagine, or glutamine at a position corresponding to position 72 as shown in SEQ ID NO: 1 (or 43);
an arginine (or conservative substitution thereof), histidine (or conservative substitution thereof), or tryptophan (or conservative substitution thereof), for example, an arginine, histidine, lysine, histidine, arginine, lysine, tryptophan, phenylalanine or tyrosine at a position corresponding to position 81 as shown in SEQ ID NO: 1 (or 43);
a threonine (or conservative substitution thereof) or isoleucine (or conservative substitution thereof), for example, a threonine, alanine, serine, glycine, isoleucine, methionine, leucine, valine or cysteine at a position corresponding to position 83 as shown in SEQ ID NO: 1 (or 43); and/or
a histidine (or conservative substitution thereof), for example, a histidine, arginine or lysine at a position corresponding to position 85 as shown in SEQ ID NO: 1 (or 43).

Preferably, the BTN2A1 binding peptide according to this disclosure comprises:
a threonine or leucine at a position corresponding to position 18 as shown in SEQ ID NO: 1;
an arginine or histidine at a position corresponding to position 20 as shown in SEQ ID NO: 1;
a glutamic acid or conservative substitution thereof chosen from aspartic acid, or leucine at a position corresponding to position 70 as shown in SEQ ID NO: 1;
an aspartic acid or asparagine, at a position corresponding to position 72 as shown in SEQ ID NO: 1;
a threonine or conservative substitution thereof chosen from alanine, serine and glycine, or a histidine; or tryptophan or phenylalanine at a position corresponding to position 81 as shown in SEQ ID NO: 1;
a threonine or conservative substitution thereof chosen from alanine, serine and glycine or isoleucine or conservative substitution thereof chosen from methionine, leucine, valine and cysteine at a position corresponding to position 83 as shown in SEQ ID NO: 1; and/or
a histidine at a position corresponding to position 85 as shown in SEQ ID NO: 1.

The (human) BTN2A1 binding peptide according to this disclosure preferably is a (human) T-cell receptor γ-chain (variable) domain, preferably a (human) T-cell receptor γ9-chain (variable) domain.

Most natural T cell receptors (TCR) comprise complete alpha (α) and beta (β) chains, but a minority of natural immune cells express an alternate receptor, formed by complete gamma (γ) and delta (δ) chains. TCR chains are typically composed of two extracellular domains: a Variable (V) domain and a Constant (C) domain, both of Immunoglobulin superfamily (IgSF) forming antiparallel β-sheets. The Constant domain is proximal to the cell membrane, followed by a transmembrane domain and a short cytoplasmic tail, while the Variable domain can bind an antigen or target moiety. The Variable domains of both the TCR α-chain and β-chain, or both the TCR γ-chain and δ-chain, each may have three hypervariable or complementarity determining regions (CDRs).

The BTN2A1 binding peptide according to this disclosure may be comprised in an (exogeneous) immune receptor or extracellular domain thereof, for example, a human (exogeneous) immune receptor or extracellular domain thereof. The immune receptor may be a T cell receptor or chimeric antigen receptor, preferably a γδ T-cell receptor or extracellular domain thereof. The γ(9)-chain (variable) domain comprising the BTN2A1 binding peptide of this disclosure may pair with any 61-8 chain.

This disclosure also provides for a Butyrophilin Subfamily 3 Member A1 (BTN3A1) and/or BTN3A2 and/or BTN3A3 binding peptide comprising an amino acid sequence that binds BTN3A1 and/or BTN3A2 and/or BTN3A3, wherein the amino acid sequence has at least 70, 75, 80, 85, 90, 95, 99, 100% sequence identity with SEQ ID NO:52 (and/or SEQ ID NO: 53, 54, 55, 56), wherein the amino acid sequence comprises:
a valine (or conservative substitution thereof), methionine (or conservative substitution thereof), alanine (or conservative substitution thereof), isoleucine (or conservative substitution thereof), leucine (or conservative substitution thereof), serine (or conservative substitution thereof) or threonine (or conservative substitution thereof), preferably a valine (or conservative substitution thereof), methionine (or conservative substitution thereof), alanine (or conservative substitution thereof) at a position corresponding to position 31 as shown in SEQ ID NO: 52 (and/or SEQ ID NO: 53, 54, 55, 56); and/or
a serine (or conservative substitution thereof) or alanine (or conservative substitution thereof) at a position corresponding to position 53 as shown in SEQ ID NO: 52 (and/or SEQ ID NO: 53, 54, 55, 56),
wherein preferably the BTN3A1/A2/A3 binding peptide is a T-cell receptor δ-chain domain, preferably a T-cell receptor δ2-chain domain.

In addition or alternatively, the modification at position 53 as shown in SEQ ID NO: 52 (and/or SEQ ID NO: 53, 54, 55, 56) is chosen from preferably lysine (K) to alanine (A) substitution, lysine (K) to cysteine (C), lysine (K) to methionine (M), a lysine (K) to serine (S) substitution, a lysine (K) to tryptophan (W) substitution, lysine (K) to valine (V), or a lysine (K) to proline (P) substitution.

The BTN3A1/A2/A3 binding peptide according to this disclosure may provide for enhanced binding affinity to BTN3A1/A2/A3 and/or may allow for more potent T cell activation and killing of tumor cells, for example, through GABs or TEGs.

The BTN3A1/A2/A3 binding peptide according to this disclosure and the BTN2A1 binding peptide according to this disclosure may be combined, such as in an (exogenous) immune receptor, T cell receptor or chimeric antigen receptor, a γδ T-cell receptor or extracellular domain thereof, more preferably a 7962 T-cell receptor or extracellular domain thereof, wherein preferably the BTN2A1 binding peptide is a T-cell receptor γ-chain domain, preferably a T-cell receptor γ9-chain domain and/or the BTN3A1/A2/A3 binding peptide is a T-cell receptor δ-chain domain, preferably a T-cell receptor δ2-chain domain.

Preferably, the (exogenous) immune receptor, the T cell receptor or chimeric antigen receptor, preferably γδ T-cell receptor or extracellular domain thereof as disclosed herein, is capable of binding, or binds, a tumor cell, e.g., an antigen present on the surface of a tumor cell.

An exogenous immune receptor according to the disclosure is preferably defined as not being an endogenous T cell receptor. For example, an exogenous immune receptor may be a particular selected γδ T cell receptor that is useful in the treatment of a cancer. The sequence may be similar to an endogenous γδ T cell receptor. The difference being that the exogenous immune receptor has been purposively selected for a specific target e.g., an antigen present on the surface of a tumor cell. The exogenous immune receptor is e.g., expressed from a transgene construct and not from endogenous loci. An exogenous immune receptor according to the disclosure may be of a different origin, i.e., from another species, as compared to the origin of the T cells that were engineered to provide for the engineered T cells with exogenous immune receptors. An exogenous immune receptor may be of the same origin, i.e., from the same species, as compared to the origin of the T cells that were engineered to provide for the engineered T cells with exogenous immune receptors. An exogenous immune receptor may also be an engineered γδ T cell receptor or an engineered ap T cell receptor.

Any of the immune receptors according to this disclosure may be a chimeric antigen receptor (CAR). Chimeric antigen receptors (CARs) are recombinant receptors that combine the specificity of an antigen-specific antibody with the T-cell's activating functions [42]. A CAR may be a fusion molecule between an antibody and a trans-membrane domain allowing expression of an antibody at the cell surface of an immune cell as well as signaling into the cell.

In one aspect of the disclosure, any of the immune receptors according to this disclosure may be selected from the group consisting of an (engineered) γδ T cell receptor, an (engineered) αβ T cell receptor, or a chimeric antigen receptor (CAR).

In particular, the BTN2A1 binding peptide according to this disclosure may be a (human) 7962 T-cell receptor or extracellular domain thereof.

This disclosure also provides for a combination or construct comprising:
i) γδ T-cell receptor or extracellular domain thereof as disclosed herein (comprising the BTN2A1 binding peptide according to this disclosure and/or BTN3A1/A2/A3 binding peptide according to this disclosure); and
ii) a toxin and/or a label.

The toxin preferably is any compound or combination of compounds effective to kill a cancer cell or an infected cell. The toxin may, for example, be a diphtheria toxin, pseudomonas toxin, and/or saporin. The toxin and/or the label may be fused to the γδ T-cell receptor or extracellular domain thereof, for example, via a linker.

The label may be any label useful in diagnostic setting. The label may allow for visualizing binding of the BTN2A1 binding peptide, to BTN2A1 and/or binding of the BTN3A1/A2/A3 binding peptide, to BTN3A1/A2/A3. The label may, for example, be a fluorophore. Fluorophores are very sensitive and generally do not affect the properties of the target molecule. The process may involves the binding of the fluorophore via the BTN2A1/BTN3A1/A2/A3 peptide of this disclosure to a BTN2A1/BTN3A1/A2/A3 protein as expressed e.g., by a cancer cell or infected cell. When the binding is complete, the fluorescence can be viewed by excitation, through a fluorescent microscope, for example. Fluorescent labeling can be used in assays such as: ELISA, FISH, and fluorescent microscopy. The label may be linked to the BTN2A1/BTN3A1/A2/A3 peptide of this disclosure by means of any linker.

This disclosure further provides a construct comprising:
i) γδ T-cell receptor or extracellular domain thereof as disclosed herein (comprising the BTN2A1 binding peptide according to this disclosure and/or BTN3A1/A2/A3 binding peptide according to this disclosure); and
ii) an (effector) cell binding domain, preferably an immune cell binding domain, preferably B-cell binding domain, macrophage binding domain or fibroblast binding domain, more preferably a T-cell binding domain and/or Natural Killer (NK) cell-binding domain.

The γδ T-cell receptor or extracellular domain thereof preferably is a 7962 T-cell receptor or extracellular domain thereof. As described herein, the term “extracellular domain” of a gamma or delta TCR chain comprises the V gamma and extracellular part of the C gamma domains, or the V delta and extracellular part of the C delta domains.

This construct combines (low) affinity TCR interaction with its ligand on tumor cells with (high) affinity interaction with e.g., T lymphocytes and/or with NK cells, preferably by binding to CD3 on T lymphocytes and/or CD 16 on NK cells. This allows first the bispecific construct to bind to a T and/or NK cell and then later to recruit this cell to a tumor. This concept may be elaborated by generating trispecific constructs for which tumor binding depends on γδTCR and a second molecule like a checkpoint ligand.

The construct according to the disclosure can attach to infected cells or cancer cells, as is indicated herein, and couple to immune cells, e.g., T-cells and/or Natural Killer (NK) cells to thereby elucidate an immune response against the infected cells or cancer cells that will reduce or even eliminate the cells. The small size of a construct according to the disclosure, together with the non-cellular nature, render the construct as an ideal treatment tool for infectious diseases and cancer. These constructs provide great promise for treatment of cancer and infectious diseases.

Fusion of the extracellular domain of a γδTCR as tumor binding domain to, for example, an anti-CD3 scFv can effectively target T cells to tumor cells without the need for engineering TEGs. γδTCR anti-CD3 bispecific molecules (abbreviated as GABs) can redirect CD 3+ effector cells toward several tumor cell lines of both hematologic and solid origin and preserve the mode of action of tumor recognition described for a particular y9δ2TCR [43, 44, 45], thereby opening a new universe of antigens to the bispecific format.

Accordingly, in the construct as described above:
the T-cell binding domain may bind cluster of differentiation 3 (CD3), CD4, CD8, CD 16, CD56, CD103, CD134, CD154 and/or CD314; and/or is a single chain Fv anti-CD3, CD4, CD8, CD 16, CD56, CD103, CD134, CD154; and/or
the Natural Killer (NK) cell-binding domain may bind CD16, NKG2D, NKp30, NKp44, NKp46, and/or DNAM, and/or is a single chain Fv anti-CD16, NKG2D, NKp30, NKp44, NKp46, and/or DNAM.

In addition or alternatively, in the construct, the binding domain might be modified so as to inhibit T cell activation. For example, the construct may bind inhibitory domain(s) of the T cell. Specifically, in the construct described above, the T cell binding domain may bind PD1 (expressed on the surface of T cells), e.g., to reduce T cell activation. This embodiment may be useful in prevention or treatment of autoimmune disease, e.g., wherein it is desired to block T cell activity and not enhance it (e.g., gdT cell or abT cell).

Accordingly, in the construct as described above:
the T-cell binding domain may bind PD1, LAG3, CTLA4, TIGIT, CD96, BTLA, VISTA, TIM3, LAIR1, (inhibitory) KIR, CD160 and/or immune receptor with an intracellular ITIM or ITSM motif; and/or may be a single chain Fv anti-PD1, LAG3, CTLA4, TIGIT, CD96, BTLA, VISTA, TIM3, LAIR1, (inhibitory) KIR, CD160 and/or immune receptor with an intracellular ITIM or ITSM motif binding domain; and/or
the Natural Killer (NK) cell binding domain may bind NKG2A, CD96, TIGIT, (inhibitory) KIR, PD1, TIM3, LAG3, CD 112R, CD160, LAIR1 and/or immune receptor with an intracellular ITIM or ITSM motif and/or may be a single chain Fv anti-NKG2A, CD96, TIGIT, (inhibitory) KIR, PD1, TIM3, LAG3, CD 112R, CD160, LAIR1 and/or immune receptor with an intracellular ITIM or ITSM motif binding domain.

The γδ T-cell receptor (or extracellular domain thereof) and the immune cell-, T-cell- and/or Natural Killer (NK) cell-binding domain are preferably fused through a linker or linking group, which preferably provides conformational flexibility so that the extracellular domain of a gamma-delta TCR can interact with its epitope, while the T-cell- and/or NK cell binding domain can interact with its cognate epitope. A preferred linker group is a linker polypeptide comprising from 1 to 60 amino acid residues, preferably from 5 to 40 amino acid residues, most preferred about 15 amino acid residues such as 10 amino acid residues, 11 amino acid residues, 12 amino acid residues, 13 amino acid residues, 14 amino acid residues, 15 amino acid residues, 16 amino acid residues, 17 amino acid residues, 18 amino acid residues, 19 amino acid residues or 20 amino acid residues. Some preferred examples of such amino acid sequences include Gly-Ser linkers, for example, of the type (Glyx Sery)z such as, for example, (Gly4 Ser)3, (Gly4 Ser)7 or (Gly3 Ser2)3, as described in WO 99/42077, and the GS30, GS15, GS9 and GS7 linkers described in, for example, WO06/040153 and WO 06/122825, as well as hinge-like regions, such as the hinge regions of naturally occurring heavy chain antibodies or similar sequences (such as described in WO 94/04678). A most preferred linker is a (Gly4 Ser)3 linker.

The immune cell-, T-cell- and/or Natural Killer (NK) cell-binding domain preferably is an antibody, preferably a single heavy chain variable domain antibody such as a camelid VHH, a shark immunoglobulin-derived variable new antigen receptor, a scFv, a tandem scFv, a scFab, an improved scFab [58], or an antibody mimetic such as a designed ankyrin repeat protein, a binding protein that is based on a Z domain of protein A, a binding protein that is based on a fibronectin type III domain, engineered lipocalin, and a binding protein that is based on a human Fyn SH3 domain [59]; [60]. For example, Liao et al, 2000 [61] describe single-chain antibodies (scFv) against CD3 that are expressed on the plasma membrane of tumor cells. In addition, single chain antibodies against CD3 are commercially available, for example, from Creative Biolabs.

A preferred single chain antibody against CD3 that is present in the (bispecific) construct according to the disclosure comprises a single chain Fv anti-CD3 binding domain. The single chain Fv anti-CD3 binding domain preferably is derived from a chimeric mouse-human OKT3 antibody, as is, for example, described in [62]. A preferred scFv derived from the OKT3 antibody has been described [63]; [64].

The construct according to this disclosure preferably is a bispecific fusion protein, e.g., the construct comprising i) γδ T-cell receptor or extracellular domain thereof as disclosed herein (comprising the BTN2A1 binding peptide according to this disclosure and/or BTN3A1/A2/A3 binding peptide according to this disclosure); and ii) a toxin and/or a label, or more preferably an (effector) cell binding domain, preferably an immune cell binding domain, more preferably a T-cell binding domain and/or Natural Killer (NK) cell-binding domain. In addition or alternatively, the γδ T-cell receptor or extracellular domain thereof is fused to the T-cell- and/or Natural Killer (NK) cell-binding domain.

The construct of this disclosure can combine tumor targeting with immune cell recruitment and thus can avoid a major drawback of engineered immune cells such as CAR-T and TEGs, which relates to the rather challenging logistic for such advanced therapy medicinal products (ATMPs). Generating ATMPs is an individualized, cumbersome, and costly process that in most cases takes week and can associate with production failures [65].

A preferred recombinant bispecific protein according to the disclosure comprises the extracellular domains of a gamma delta TCR, preferably gamma 9 delta 2 TCR. A preferred recombinant bispecific protein according to the disclosure comprises the extracellular domains of a TCR gamma chain, preferably gamma 9 (comprising the BTN2A1 peptide as disclosed herein disclosure and/or BTN3A1/A2/A3 binding peptide disclosed herein), that is coupled at its C-terminus to a CD3-binding domain, preferably a scFv derived from the OKT3 antibody as e.g., described in [64], and preferably an extracellular domain of a delta TCR, preferably a delta 2 TCR. In addition or alternatively, the extracellular domain of gamma and/or delta TCR, preferably of the delta TCR, preferably of the delta 2 TCR, may be fused at the N-terminus or C-terminus to the extracellular domain of checkpoint-related molecule such as the extracellular domain of a PD-1 receptor.

This disclosure also provides for an nucleic acid or nucleic acid combination encoding the BTN2A1 binding peptide according to the disclosure and/or BTN3A1/A2/A3 binding peptide according to this disclosure or (any of the) construct(s) according to the disclosure. The nucleic acid may be comprised in a vector and/or comprised in a cell that may or may not be an immune cell. Preferably, the nucleic acid is expressed in the cell.

An immune receptor (or extracellular domain thereof) according to this disclosure may be e.g., a gamma delta T cell receptor (or extracellular domain thereof) that comprises a first chain that is gamma and a second chain that is the delta chain. These may be provided on a single nucleic acid or on two separate nucleic acids. A first nucleic acid encoding the first chain, and a second nucleic acid encoding the second chain, or a single nucleic acid encoding both the first and second chains. The nucleic acid or nucleic acids may be DNA or RNA. As long as when it is introduced in a cell and expressed such that the amino acid sequence of the exogenous immune receptor it encodes is expressed on the surface of the cell.

Preferably, in one embodiment, the nucleic acid encoding the immune receptor (or extracellular domain thereof) encodes an immune receptor (or extracellular domain thereof) wherein the different chains, e.g., gamma and delta chains, are expressed as a single translated protein product that comprising the F2A or T2A peptide linker sequence in between the encoding sequences of the both chains resulting in self-cleavage of the translated protein such that separate chains are formed.

The nucleic acid or nucleic acids that encode the immune receptor (or extracellular domain thereof) according to the disclosure may be mRNA that can be translated directly in the immune receptor (or extracellular domain thereof) when introduced in the cytoplasm of a T cell, e.g., via transfection. Preferably, the nucleic acid (or nucleic acids) encoding e.g., a T-cell receptor chain is comprised in a genetic construct. The genetic construct (or constructs) may allow the expression of mRNA that encodes the immune receptor (or extracellular domain thereof) such that it is expressed on the surface of the engineered T cell. A genetic construct may be comprised in a DNA vector or in a viral vector. Introduction of the nucleic acid or nucleic acids may be via transfection or transduction methods depending on what type of nucleic acid or nucleic acids are used. It is understood that depending on what type of genetic construct or constructs are used, the genetic construct may consist of DNA or RNA. For example, when a genetic construct is incorporated in a retroviral or lentiviral vector the genetic construct is comprised in an RNA vector genome (i.e., the sequence that encodes the genetic construct). Retroviral and lentiviral vectors are well known in the art having an RNA genome that, when entered in a cell, is reverse transcribed into DNA that is subsequently integrated into the host genome. Reverse transcription thus results in the genetic information, i.e., the genetic construct, being transformed from RNA into double stranded DNA thereby allowing expression therefrom. Integration is advantageous as it allows for proliferation of transduced cells while maintaining the viral vector genome comprising the genetic construct. A genetic construct may also be comprised in a DNA vector, e.g., plasmid DNA. A suitable DNA vector may be a transposon. Suitable transposon systems (e.g., class I or class II based) are well known in the art. As said, when an immune receptor comprises two chains, e.g., a gamma and delta T cell receptor chain, two separate genetic constructs can be provided e.g., on a single or two separate retroviral or DNA vectors. Alternatively, a single genetic construct may also express a single mRNA encoding the two chains. Such an mRNA may encode the two chains separately, e.g., via an IRES, or via using self-cleavable peptide sequences as described herein.

The nucleic acid or nucleic acids that are used may provide for expression of the encoded immune receptor (or extracellular domain thereof). This is achieved e.g., via high levels of expression of the immune receptor (or extracellular domain thereof) by using e.g., a strong promoter.

Accordingly, this disclosure also provides for a cell expressing the BTN2A1 binding peptide according to the disclosure and/or BTN3A1/A2/A3 binding peptide according to this disclosure, the construct according to the disclosure, and/or the nucleic acid or nucleic acid combination according to the disclosure as described above.

The cell may be a bacterial cell, for example, an Escherichia coli cell, or a eukaryotic cell such as a fungal cell including a yeast cell, for example, Saccharomyces cerevisiae or a methylotrophic yeast such as Pichia pastoris, or a mammalian cell. The eukaryotic cell preferably is a cell that can easily be infected and/or transfected using standard methods known to the skilled person, such as, for example, yeast cells and chicken fibroblast cells. The eukaryotic cell preferably is an insect cell or a mammalian cell. Suitable insect cells comprise, for example, ovarian Spodoptera frugiperda cells such as Sf9 and Sf21, Drosophila Schneider 2 cells and Aedes albopictus C6/36 cells. Suitable mammalian cells comprise, for example, Baby Hamster Kidney cells, Human Embryonic Kidney cells such as HEK293 and freestyle HEK293F™ cells (Thermo Fisher Scientific), VERO cells, MDCK cells, CHO cells, HeLa and PER.C6 cells [66]. Preferred cells are Human Embryonic Kidney cells such as HEK293 and freestyle HEK293F™ cells.

The cell expressing the BTN2A1 binding peptide according to the disclosure and/or BTN3A1/A2/A3 binding peptide according to this disclosure may be an immune cell, more preferably a human T cell or human NK cell, more preferably an αβ T-cell or a γδ T-cell. The immune cells according to this disclosure may be immune cells that are engineered to comprise and preferably express an exogenous immune receptor. The immune cell according to this disclosure may be a human immune cell, preferably a human T cell or human NK cell. The exogenous immune receptor may have the same function as a corresponding endogenous T cell receptor with regard to antigen recognition and T cell action. Non-engineered immune cells are cells that express an endogenous immune receptor, i.e., T cell receptor.

Cells such as immune cells, e.g., T cells or NK cells of a subject, may be isolated or established immune cell lines may be used. The subject may suffer from cancer (a patient) or may be a healthy subject. These immune cells can be genetically modified in vitro to express the immune receptor (or extracellular domain thereof) as disclosed herein. These engineered cells may be activated and expanded in vitro to a therapeutically effective population of expressing cells. In cellular therapy these engineered cells may be infused to a recipient in need thereof as a pharmaceutical composition. The infused cells in the recipient may be able to kill (or at least stop growth of) cancerous cells expressing the antigen that is recognized by the immune receptor as disclosed herein. The recipient may be the same subject from which the cells were obtained (autologous cell therapy) or may be from another subject of the same species (allogeneic cell therapy).

Also envisaged are αβ T-cells with γδ TCRs (TEGs) according to this disclosure, which combine the strong proliferation capacity of αβ T cells (which are active even in late stage cancer patients, see [46]), with the broad tumor-reactivity of γδ TCRs.

This disclosure further relates to a method of producing the BTN2A1 binding peptide according to the disclosure and/or BTN3A1/A2/A3 binding peptide according to this disclosure, or the construct according to the disclosure, wherein the method comprises expressing the BTN2A1 binding peptide according to the disclosure and/or BTN3A1/A2/A3 binding peptide according to this disclosure, or the construct according to the disclosure in a host cell thereby producing the BTN2A1 binding peptide according to the disclosure and/or BTN3A1/A2/A3 binding peptide according to this disclosure, or the construct according to the disclosure. The BTN2A1 binding peptide according to the disclosure and/or BTN3A1/A2/A3 binding peptide according to this disclosure, or the construct according to the disclosure may be secreted into the growth medium of the host cell.

Also provided is a pharmaceutical composition comprising the BTN2A1 binding peptide according to this disclosure and/or BTN3A1/A2/A3 binding peptide according to this disclosure (or a y6 TCR or extracellular domain thereof comprising said), the construct according to this disclosure, or the cell according to this disclosure. A pharmaceutical composition preferably comprises a pharmaceutically acceptable carrier. A carrier, as used herein, means a non-toxic material that does not interfere with the effectiveness of the biological activity of the active ingredient. The term “physiologically acceptable” refers to a non-toxic material that is compatible with a biological system such as a cell, cell culture, tissue, or organism. The characteristics of the carrier will depend on the route of administration.

Formulations comprising therapeutically effective population(s) of cells or constructs according to this disclosure may include pharmaceutically acceptable excipient(s) (carrier or diluents). Excipients included in the formulations will have different purposes depending, for example, on the nature of the construct, the (sub)population of immune cells used, and the mode of administration. Examples of generally used excipients include, without limitation: saline, buffered saline, dextrose, water-for-injection, glycerol, ethanol, and combinations thereof, stabilizing agents, solubilizing agents and surfactants, buffers and preservatives, tonicity agents, bulking agents, and lubricating agents.

The formulations comprising therapeutically effective population(s) of cells or constructs according to this disclosure may be administered to a subject using modes and techniques known to the skilled artisan. Exemplary modes include, but are not limited to, intravenous injection. Other modes include, without limitation, intratumoral, intradermal, subcutaneous (s.c, s.q., sub-Q, Hypo), intramuscular (i.m.), intraperitoneal (i.p.), intra-arterial, intramedulary, intracardiac, intra- articular (joint), intrasynovial (joint fluid area), intracranial, intraspinal, and intrathecal (spinal fluids).

In general, formulations may be administered that comprise between about 1×10⁴ and about 1×10¹⁰ immune cells, or between 0.1-10, or 1-100, 10-1000 mg construct. In most cases, the formulation may comprise between about 1×10⁵ and about 1×10⁹ immune cells, from about 5×10⁵ to about 5×10⁸ immune cells, or from about 1×10⁶ to about 1×10⁷ immune cells. A physician may ultimately determine appropriate dosages to be used.

The BTN2A1 binding peptide according to this disclosure and/or BTN3A1/A2/A3 binding peptide according to this disclosure (or a y8 TCR or extracellular domain thereof comprising the peptide), the construct according to this disclosure, or the cell according to this disclosure can be administered by injection or by (gradual) infusion over time. The administration of the construct preferably is parenteral such as, for example, intravenous, intraperitoneal, intranasal, or intramuscular. Preparations for parenteral administration include sterile aqueous or non-aqueous solutions suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media.

Parenteral vehicles include sodium chloride solution, Ringer's dextrose, dextrose and sodium chloride, lactated Ringer's or fixed oils. Intravenous vehicles include fluid and nutrient replenishes, electrolyte replenishes (such as those based on Ringer's dextrose), and the like. Preservatives and other additives may also be present such as, for example, antimicrobials, anti-oxidants, chelating agents, and inert gases and the like.

This disclosure particularly provides for the BTN2A1 binding peptide according to the disclosure and/or BTN3A1/A2/A3 binding peptide according to this disclosure (or (γδ)TCT or extracellular domain thereof comprising the peptide), any one of the constructs according to this disclosure, or the cell according to this disclosure, for use in therapy, preferably for use in the treatment of a cancer and/or an infection (such as infectious disease).

Accordingly, there is provided for a method for treatment of a cancer and/or an infection (such as infectious disease), comprising administering an effective amount of the BTN2A1 binding peptide according to the disclosure and/or BTN3A1/A2/A3 binding peptide according to this disclosure (or (γδ)TCT or extracellular domain thereof comprising the peptide), any one of the constructs according to this disclosure, or the cell according to this disclosure, e.g., to a subject in need thereof. Preferably, the subject is human.

As will be clear, the γδTCR or extracellular domain thereof comprising the BTN2A1 binding peptide according to the disclosure and/or BTN3A1/A2/A3 binding peptide according to this disclosure, preferably is capable of binding, or binds, a tumor cell, e.g., an antigen present on the surface of a tumor cell.

Occasional off-target toxicity (by the tight binding of the BTN2A1/BTN3A1/A2/A3 binding peptide or the Vγ9/Vδ2 chain comprising said to BTN2A1/BTN3A1/A2/A3 on healthy tissue) can be overcome by lowering dose of the BTN2A1/BTN3A1/A2/A3 binding peptide (or the γδTCR or extracellular domain thereof comprising said).

The use or method according to this disclosure may not require co-administration of aminobisphosphonates, such as pamidronate or zoledronate, to increase the intracellular levels of phosphoantigens, e.g., in tumor cells or infected cells, or at least less aminobisphosphonates, such as pamidronate or zoledronate, are needed in such co-administration, such as at most 10, 9, 8, 7, 6, 5, 4, 3, 2, 1 mg is applied in the method or use. One of the major obstacles in current Vγ9Vδ2 TCR based therapies is the low levels of phosphoantigens in tumor cells, leading to a suboptimal activation of Vγ9Vδ2 T cells or Vγ9Vδ2-TCR based therapies. This disclosure can overcome this problem by enhancing binding to BTN2A1/BTN3A1/A2/A3.

Therapy in this disclosure may involve prophylactic administration or therapeutic administration in humans that are suffering from e.g., a cancer or an infectious disease. Thus, the BTN2A1 binding peptide according to the disclosure and/or BTN3A1/A2/A3 binding peptide according to this disclosure (or (γδ)TCT or extracellular domain thereof comprising said), any one of the constructs according to this disclosure, or the cell according to this disclosure, may be administered to an individual that is suspected of suffering from a cancer or an infection, or may be administered to an individual already evidencing active infection or cancer in order to lessen signs and symptoms of the cancer or infection.

For example, in the treatment of leukemia, a patient undergoing an allogeneic stem cell transplantation may also benefit from an infusion of the BTN2A1 binding peptide according to the disclosure and/or BTN3A1/A2/A3 binding peptide according to this disclosure (or (γδ)TCT or extracellular domain thereof comprising the peptide), any one of the constructs according to this disclosure, or the cell according to this disclosure. This way, elimination of leukemia may be promoted.

This disclosure also provides for the BTN2A1 binding peptide according to the disclosure and/or BTN3A1/A2/A3 binding peptide according to this disclosure for use in a diagnostic method, preferably wherein the use is for determining cellular BTN2A1/BTN3A1/A2/A3 expression in a sample.

In particular, disclosed is a method or use for determining cellular BTN2A1/BTN3A1/A2/A3 expression in a sample, wherein the method or use comprises:
a) providing a sample comprising cells obtained from a subject (e.g., tumor sample);
b) combining the cells with the BTN2A1 binding peptide according to this disclosure and/or BTN3A1/A2/A3 binding peptide according to this disclosure (under conditions suitable for binding); and
c) determining binding of the BTN2A1 binding peptide and/or BTN3A1/A2/A3 binding peptide to the cells, thereby determining cellular BTN2A1/BTN3A1/A2/A3 expression in the sample.

The method or use can be useful for determining e.g., susceptibility for BTN2A1 binding peptide and/or BTN3A1/A2/A3 binding peptide therapeutic strategies according to this disclosure, e.g., in a patient suffering from cancer and/or infection (infectious disease).

In particular, this disclosure provides for an in vitro method for determining cellular BTN2A1/BTN3A1/A2/A3 expression in a sample, the method comprising:
a) providing a sample comprising cells obtained from a subject (e.g., tumor sample), preferably a cancer patient or infectious disease patient;
b) combining the cells with the BTN2A1 binding peptide according to this disclosure and/or BTN3A1/A2/A3 binding peptide according to this disclosure (under conditions suitable for binding); and
c) determining binding of the BTN2A1 binding peptide and/or BTN3A1/A2/A3 binding peptide to the cells, thereby determining cellular BTN2A1/BTN3A1/A2/A3 expression in the sample, preferably wherein the binding is indicative of cellular BTN2A1/BTN3A1/A2/A3 expression, and absence of the binding is indicative of no cellular BTN2A1/BTN3A1/A2/A3 expression.

Step c) may involve determining an extent of binding of the BTN2A1 binding peptide and/or BTN3A1/A2/A3 binding peptide to the cells, thereby determining an extent cellular BTN2A1/BTN3A1/A2/A3 expression in the sample, preferably wherein the extent of binding is indicative of the extent of cellular BTN2A1/BTN3A1/A2/A3 expression.

This disclosure also provides for a p chain peptide that has at least 30, 40, 50, 60, 70, 80, 90, 95, 99, 100% sequence identity with any one of SEQ ID NOs: 12-42, for example, SEQ ID NO: 13, 14, 15, 16, 17, 18, 19 and/or 20, wherein the amino acid sequence comprises:
an amino acid other than arginine at a position corresponding to position 69 as shown in SEQ ID NO: 12 (or an amino acid other than arginine at a position corresponding to position 69 in any one of SEQ ID NO: 13-42, for example, SEQ ID NO: 13, 14, 15, 16, 17, 18, 19 and/or 20).

Position corresponding to position 69 in SEQ ID NO:12, relates to:
position corresponding to position 69 in SEQ ID NO:12
position corresponding to position 68 in SEQ ID NO: 13
position corresponding to position 68 in SEQ ID NO: 14
position corresponding to position 68 in SEQ ID NO: 15
position corresponding to position 68 in SEQ ID NO:16
position corresponding to position 68 in SEQ ID NO: 17
position corresponding to position 68 in SEQ ID NO: 18
position corresponding to position 67 in SEQ ID NO: 19
position corresponding to position 68 in SEQ ID NO:20
position corresponding to position 69 in SEQ ID NO:21
position corresponding to position 69 in SEQ ID NO:22
position corresponding to position 69 in SEQ ID NO:23
position corresponding to position 69 in SEQ ID NO:24
position corresponding to position 69 in SEQ ID NO:25
position corresponding to position 69 in SEQ ID NO:26
position corresponding to position 69 in SEQ ID NO:27
position corresponding to position 68 in SEQ ID NO:28
position corresponding to position 68 in SEQ ID NO:29
position corresponding to position 68 in SEQ ID NO:30
position corresponding to position 69 in SEQ ID NO:31
position corresponding to position 69 in SEQ ID NO:32
position corresponding to position 69 in SEQ ID NO:33
position corresponding to position 69 in SEQ ID NO:34
position corresponding to position 68 in SEQ ID NO:35
position corresponding to position 68 in SEQ ID NO:36
position corresponding to position 68 in SEQ ID NO:37
position corresponding to position 68 in SEQ ID NO:38
position corresponding to position 68 in SEQ ID NO:39
position corresponding to position 68 in SEQ ID NO:40
position corresponding to position 70 in SEQ ID NO:41
position corresponding to position 68 in SEQ ID NO:42

The β chain peptide of this disclosure may have antigen binding functionality, such as tumor antigen binding functionality.

It was found that mutating the amino acid at the position as naturally occurring in the β chain peptide surprisingly increases stability of the peptide, and can increase production yield (e.g., in grams of peptide or construct comprising said) per volume of culture by more than 2-fold.

In the β chain peptide of this disclosure, the amino acid other than arginine at a position corresponding to position 69 as shown in SEQ ID NO: 12 (or an amino acid other than arginine at a position corresponding to position 69 in any one of SEQ ID NO: 13-42) may be tyrosine or conservative substitution thereof. Alternatively, it may be phenylalanine or conservative substitution thereof, tyrosine or conservative substitution thereof, or tryptophan or conservative substitution thereof.

The β chain peptide of this disclosure may be a T-cell receptor 3-chain (variable) domain, preferably a T-cell receptor 06-chain (variable) domain. In addition or alternatively, the β chain peptide of this disclosure may be comprised in an (exogeneous) immune receptor or extracellular domain thereof, preferably a αβ T-cell receptor or extracellular domain thereof.

This disclosure also provides a nucleic acid or nucleic acid combination encoding the β chain peptide of this disclosure. Further, provided is a cell expressing the β chain peptide of this disclosure, wherein the cell preferably is an immune cell, more preferably a human T cell or human NK cell, more preferably an αβ T-cell or a γδ T-cell.

This disclosure further envisages a method of producing the β chain peptide of this disclosure, comprising expressing the β chain peptide of this disclosure, thereby producing the β chain peptide of this disclosure. The β chain peptide of this disclosure may be secreted into the growth medium of the host cell.

Also foreseen is a pharmaceutical composition comprising the β chain peptide of this disclosure and/or the β chain peptide of this disclosure for use in therapy, preferably for use in the treatment of a cancer and/or an infection. Such treatment may involve administering p chain peptide of this disclosure, e.g., in an effective amount, to a subject in need thereof. Preferably, the subject is human.

In the context of this disclosure, the term “(poly)peptide” is equivalent to the term “protein” and/or a (poly)peptide may be part of a protein, i.e., comprised in a protein or protein domain. A (poly)peptide has a particular amino acid sequence. A “variant” of the polypeptide of this disclosure preferably has an amino acid sequence that has at least 25% sequence identity to a reference polypeptide. A (poly)peptide of the disclosure is isolated when it is no longer in its natural environment. A peptide according to this disclosure may have a length of between 1-500 bp, 10⁻⁵⁰⁰ bp, 50-250 bp, or at least 5, 10, 20, 30, 40, 50 bp and/or at most 100, 150, 200, 300, 400, 500, 1000 bp.

T cells, or T lymphocytes, belong to a group of white blood cells named lymphocytes, which play a role in cell-mediated immunity. T cells originate from hematopoietic stem cells in the bone marrow, mature in the thymus (that is where the T is derived from), and gain their full function in peripheral lymphoid tissues. During T-cell development, CD4⁻CD8⁻ T-cells (negative for both the CD4 and CD8 co-receptor) are committed either to an ap (alpha beta) or γδ (gamma delta) fate as a result of an initial R or 6 TCR gene rearrangement. Cells that undergo early β chain rearrangement express a pre-TCR structure composed of a complete β chain and a pre-TCRα chain on the cell surface. Such cells switch to a CD4⁺CD8⁺ state, rearrange the TCRα chain locus, and express an αβTCR on the surface. CD4⁻CD8⁻ T cells that successfully complete the y gene rearrangement before the R gene rearrangement express a γδTCR and remain CD4⁻CD8⁻ [47]. The T cell receptor associates with the CD3 protein to form a T cell receptor complex. T cells, i.e., expressing an αβTCR or a 76TCR, express the T cell receptor complex on the cell surface. The γδT-cells constitute about 1-5% of the total population of T cells. The extracellular region of a T cell receptor chain comprises a variable region. The variable region of a T cell receptor chain three complementarity determining regions (CDR1, CDR2, CDR3) are located. These regions are in general the most variable and contribute to diversity among TCRs. CDR regions are composed during the development of a T-cell where so-called Variable-(V), Diverse-(D), and Joining-(J)-gene segments are randomly combined to generate diverse TCRs. The constant region of a T cell receptor chain, i.e., being either an alpha, beta, gamma or delta chain, does not substantially vary. Similarly, the framework regions of a T cell receptor chain, i.e., being either an alpha, beta, gamma or delta chain, do not substantially vary either.

“γδT cells” or “gamma delta T cells” represent a small subset of T cells for which the antigenic molecules that trigger their activation is largely unknown. Gamma delta T cells may be considered a component of adaptive immunity in that they rearrange TCR genes to produce junctional diversity and will develop a memory phenotype. However, various subsets may also be considered part of the innate immunity where a restricted TCR is used as a pattern recognition receptor. For example, Vγ9/Vδ2 T cells are specifically and rapidly activated by a set of non-peptidic phosphorylated isoprenoid precursors, collectively named phosphoantigens. γδT-cells may be identified using an antibody specific for the γδ T-cell receptor. Antibodies suitable for FACS are widely available. Conditions are selected, such as provided by the antibody manufacturer that allows the selection of negative and/or positive cells. Examples of antibodies that may be suitable are available from BD Pharmingen (BD, 1 Becton Drive, Franklin Lakes, NJ USA), γδTCR-APC (clone B1, #555718) or as available from Beckman Coulter, pan-76TCR-PE (clone INMU510, #IM1418U). Also, from such selected cells, the nucleic acid (or amino acid sequence) sequence corresponding to the γT cell receptor chain and/or the δT cell receptor chain may be determined. Hence, γδT cells may also be defined as being cells comprising a nucleic acid (or amino acid) sequence corresponding to a γT-cell receptor chain and/or a δ2T-cell receptor chain.

Natural Killer cells (NK cells) are defined as large granular lymphocytes (LGL) and constitute the third kind of cells differentiated from the common lymphoid progenitor generating B and T lymphocytes. NK cells are known to differentiate and mature in the bone marrow, lymph node, spleen, tonsils and thymus where they then enter into the circulation. NK cells do not express T-cell antigen receptors (TCR) or Pan T marker CD3 or surface immunoglobulins (Ig) B cell receptors, but they usually express the surface markers CD16 (FcγRIII) and CD56 in humans, NK1.1 or NK1.2 in C57BL/6 mice. Up to 80% of human NK cells also express CD8.

The term ‘conserved substitutions’ as used herein may refer to replacement of one or more amino acids in a polypeptide without substantial loss of functionality. It is common general knowledge that it is possible to substitute a certain amino acid by another one, without loss of activity of the polypeptide. For example, the following amino acids can typically be exchanged for one another:
Ala, Ser, Thr, Gly (small aliphatic, nonpolar or slightly polar residues)
Asp, Asn, Glu, Gln (polar, negatively charged residues and their amides)
His, Arg, Lys (polar, positively charged residues)
Met, Leu, Ile, Val (Cys) (large aliphatic, nonpolar residues)
Phe, Tyr, Trp (large aromatic residues)
(refer, for example, to [67]).

Preferred “substitutions” are those that are conservative, i.e., wherein the residue is replaced by another of the same general type. In making changes, the hydropathic index of amino acids may be considered (see, e.g., [68]). It is known in the art that certain amino acids may be substituted by other amino acids having a similar hydropathic index or score and still result in a polypeptide having similar biological activity. In making such changes, the substitution of amino acids whose hydropathic indices are within ±2 is preferred, those that are within ±1 are more preferred, and those within ±0.5 are even more preferred. Similarly, select amino acids may be substituted by other amino acids having a similar hydrophilicity, as set forth in U.S. Pat. No. 4,554,101. In making such changes, as with the hydropathic indices, the substitution of amino acids whose hydrophilicity indices are within +2 is preferred, those that are within ±1 are more preferred, and those within +0.5 are even more preferred.

As used herein, the term “sequence identity” refers to a measure of the identity of nucleotide sequences or amino acid sequences. In general, the sequences are aligned so that the highest order match is obtained. “Identity” per se has an art-recognized meaning and can be calculated using published techniques. See, e.g.: [48], [49], [50], [51], [52]. While there exist a number of methods to measure identity between two polynucleotide or polypeptide sequences, the term “identity” is well known to skilled artisans [53]. Methods commonly employed to determine identity or similarity between two sequences include, but are not limited to, those disclosed in [54], [55]. Methods to determine identity and similarity are codified in computer programs. For example, NCBI Nucleotide Blast with standard settings (blastn, blast.ncbi.nlm.nih.gov). Preferred computer program methods to determine identity and similarity between two sequences include, but are not limited to, GCS program package [56]. [57].

As an illustration, by a nucleotide sequence or amino acid sequence having at least, for example, 95% “identity” to a reference sequence, it is intended that the nucleotide sequence or amino acid sequence is identical to the reference sequence except that there may be up to five point mutations per each 100 nucleotides or amino acids of the reference sequence. In other words, to obtain a nucleotide sequence or amino acid sequence being at least 95% identical to a reference sequence, up to 5% of the nucleotides or amino acids in the reference sequence may be deleted and/or substituted with another nucleotide or amino acid, and/or a number of nucleotides or amino acids up to 5% of the total nucleotides or amino acids in the reference sequence may be inserted into the reference sequence. Preferably, the sequence identity refers to the sequence identity over the entire length of the sequence. It is further understood that, when referring to “sequences” herein, generally the actual physical molecules with a certain sequence of subunits (e.g., amino acids or nucleotides) are referred to.

In this disclosure and in its claims, the verb “to comprise” and its conjugations are used in their non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one.”

### Sequences

| Amino acid | Three letter code | One letter code |
|---|---|---|
| alanine | ala | A |
| arginine | arg | R |
| asparagine | asn | N |
| aspartic acid | asp | D |
| asparagine or aspartic acid | asx | B |
| cysteine | cys | C |
| glutamic acid | glu | E |
| glutamine | gln | Q |
| glutamine or glutamic acid | glx | Z |
| glycine | gly | G |
| histidine | his | H |
| isoleucine | ile | I |
| leucine | leu | L |
| lysine | lys | K |
| methionine | met | M |
| phenylalanine | phe | F |
| proline | pro | P |
| serine | ser | S |
| threonine | thr | T |
| tryptophan | trp | W |
| tyrosine | tyr | Y |
| valine | val | V |
| any amino acid | any | X |

### Overview of Vgamma 9 Sequences.

CDR regions are in italic font, important residues in bold.

### Position of Stabilizing Residue Bold Underlined (γR73Y Mutation)

SEQ ID NO: 1:
AGHLEQPQISSTKTLSKTARLECVVSXXXXXXXXVYWYRERPGEVIQ
FLVSXXXXXXXRKESGIPSGKFEVDRIPETSTSTLTIHNVEKQDIAT
YYXXXXX
(TRGV9*01 germline, used in GAB-AJ8, TEG-MNP2,
TEG-LM1)

An example sequence with specific CDR region is represented below, wherein yield affecting position 73 is underlined. Potency affecting position E22 is in dark gray and bold, important residue positions T18, R20, E70, D72, T81, T83, H85 are in dark gray, and permissive residues/non tested positions S31, G56, V58, S66, I74, K60, E76, T79 are in mid-gray.

>TRGV9*01
SEQ ID NO: 43:
AGHLEQPQIS STKTLSKTAR LECVVSGITI SATSVYWYRE RPGEVIQFLV
SISYDGTVRK ESGIPSGKFE VDRIPETSTS TLTIHNVEKQ DIATYYCALW E
(Vγ9-R73Y, used in GAB-AJ8s = GAB-AJ8_{γR73Y})
SEQ ID NO: 2
AGHLEQPQIS STKTLSKTAR LECVVSGITI SATSVYWYRE RPGEVIQFLV
SISYDGTVRK ESGIPSGKFE VDYIPETSTS TLTIHNVEKQ DIATYYCALW E
(Vγ9-E22W-R73Y, used in GAB-AJ8s_{γE22W}, GAB-A3S_{γE22W},
GAB-LM1SE22W)
SEQ ID NO: 3
AGHLEQPQIS STKTLSKTAR LWCVVSGITI SATSVYWYRE RPGEVIQFLV
SISYDGTVRK ESGIPSGKFE VDYIPETSTS TLTIHNVEKQ DIATYYCALW E
(Vγ9-R73Y-T81H, used in GAB-AJ8s_{γT81H})
SEQ ID NO: 4
AGHLEQPQIS STKTLSKTAR LECVVSGITI SATSVYWYRE RPGEVIQFLV
SISYDGTVRK ESGIPSGKFE VDYIPETSTS HLTIHNVEKQ DIATYYCALW E
(Vγ9-R73Y-T81W, used in GAB-AJ8s_{γT81W})
SEQ ID NO: 5
AGHLEQPQIS STKTLSKTAR LECVVSGITI SATSVYWYRE RPGEVIQFLV
SISYDGTVRK ESGIPSGKFE VDYIPETSTS WLTIHNVEKQ DIATYYCALW E
(Vγ9-E22W, used in T cells transduced with γδTCR MNP2γE22W
or TEG-LM1_{γE22W})
SEQ ID NO: 6
AGHLEQPQIS STKTLSKTAR LWCVVSGITI SATSVYWYRE RPGEVIQFLV
SISYDGTVRK ESGIPSGKFE VDRIPETSTS TLTIHNVEKQ DIATYYCALW E

### Sequences of Vγ9-Acceptor Cassettes

Nucleotide sequences of the Vγ9-acceptor cassettes. These cassettes were sub-cloned into the GAB expression vector using the BglII and EcoNI restriction sites. The introduced BsmBI-acceptor site is highlighted in bold.

(>Vg9_18_22 acc)
SEQ ID NO: 44
AGATCTCCGCCGCCACCATGGCATGCCCTGGCTTCCTGTGGGCACTTGTGATCTCCACCTGTCT
TGAATTTTCCATGGCCCGTACGGGCGCCGGACACCTGGAACAGCCCCAGATCAGCAGCACCAAG
ACCCTGAGCAAGTGAGACGTTTCCTGATGGACTGACGTCTCACGTGGTGTCCGGCATCACCATC
AGCGCCACCTCCGTGTACTGGTACAGAGAGAGACCCGGCGAGGTCATCCAGTTCCTGGTGTCCA
TCAGCTACGACGGCACCGTGCGGAAAGAGAGCGGCATCCCCAGCGGCAAGTTCGAGGTGGACTA
CATCCCCGAGACCAGCACCTCCACCCTGACCATCCACAACGTGGAGAAGCAGGACATCGCCACC
TACTACTGCGCCCTGTGGGAGG
(>Vg9_29_33_acc)
SEQ ID NO: 7
AGATCTCCGCCGCCACCATGGCATGCCCTGGCTTCCTGTGGGCACTTGTGATCTCCACCTGTCT
TGAATTTTCCATGGCCCGTACGGGCGCCGGACACCTGGAACAGCCCCAGATCAGCAGCACCAAG
ACCCTGAGCAAGACCGCCAGGCTGGAATGCGTGGTGTCCGGCATCTGAGACGTTTCCTGATGGA
CTGACGTCTCATCCGTGTACTGGTACAGAGAGAGACCCGGCGAGGTCATCCAGTTCCTGGTGTC
CATCAGCTACGACGGCACCGTGCGGAAAGAGAGCGGCATCCCCAGCGGCAAGTTCGAGGTGGAC
TACATCCCCGAGACCAGCACCTCCACCCTGACCATCCACAACGTGGAGAAGCAGGACATCGCCA
CCTACTACTGCGCCCTGTGGGAGG
(>Vg9_56_60_acc)
SEQ ID NO: 8
AGATCTCCGCCGCCACCATGGCATGCCCTGGCTTCCTGTGGGCACTTGTGATCTCCACCTGTCT
TGAATTTTCCATGGCCCGTACGGGCGCCGGACACCTGGAACAGCCCCAGATCAGCAGCACCAAG
ACCCTGAGCAAGACCGCCAGGCTGGAATGCGTGGTGTCCGGCATCACCATCAGCGCCACCTCCG
TGTACTGGTACAGAGAGAGACCCGGCGAGGTCATCCAGTTCCTGGTGTCCATCAGCTACGACTG
AGACGTTTCCTGATGGACTGACGTCTCAGAGAGCGGCATCCCCAGCGGCAAGTTCGAGGTGGAC
TACATCCCCGAGACCAGCACCTCCACCCTGACCATCCACAACGTGGAGAAGCAGGACATCGCCA
CCTACTACTGCGCCCTGTGGGAGG
(>Vg9_66_70_acc)
SEQ ID NO: 9
AGATCTCCGCCGCCACCATGGCATGCCCTGGCTTCCTGTGGGCACTTGTGATCTCCACCTGTCT
TGAATTTTCCATGGCCCGTACGGGCGCCGGACACCTGGAACAGCCCCAGATCAGCAGCACCAAG
ACCCTGAGCAAGACCGCCAGGCTGGAATGCGTGGTGTCCGGCATCACCATCAGCGCCACCTCCG
TGTACTGGTACAGAGAGAGACCCGGCGAGGTCATCCAGTTCCTGGTGTCCATCAGCTACGACGG
CACCGTGCGGAAAGAGAGCGGCATCCCCAGAGACGTTTCCTGATGGACTGACGTCTCAGTGGAC
TACATCCCCGAGACCAGCACCTCCACCCTGACCATCCACAACGTGGAGAAGCAGGACATCGCCA
CCTACTACTGCGCCCTGTGGGAGG
(>Vg9_72_76_acc)
SEQ ID NO: 10
AGATCTCCGCCGCCACCATGGCATGCCCTGGCTTCCTGTGGGCACTTGTGATCTCCACCTGTCT
TGAATTTTCCATGGCCCGTACGGGCGCCGGACACCTGGAACAGCCCCAGATCAGCAGCACCAAG
ACCCTGAGCAAGACCGCCAGGCTGGAATGCGTGGTGTCCGGCATCACCATCAGCGCCACCTCCG
TGTACTGGTACAGAGAGAGACCCGGCGAGGTCATCCAGTTCCTGGTGTCCATCAGCTACGACGG
CACCGTGCGGAAAGAGAGCGGCATCCCCAGCGGCAAGTTCGAGGTGTGAGACGTTTCCTGATGG
ACTGACGTCTCAACCAGCACCTCCACCCTGACCATCCACAACGTGGAGAAGCAGGACATCGCCA
CCTACTACTGCGCCCTGTGGGAGG
(>Vg9_81_85_acc)
SEQ ID NO: 11
AGATCTCCGCCGCCACCATGGCATGCCCTGGCTTCCTGTGGGCACTTGTGATCTCCACCTGTCT
TGAATTTTCCATGGCCCGTACGGGCGCCGGACACCTGGAACAGCCCCAGATCAGCAGCACCAAG
ACCCTGAGCAAGACCGCCAGGCTGGAATGCGTGGTGTCCGGCATCACCATCAGCGCCACCTCCG
TGTACTGGTACAGAGAGAGACCCGGCGAGGTCATCCAGTTCCTGGTGTCCATCAGCTACGACGG
CACCGTGCGGAAAGAGAGCGGCATCCCCAGCGGCAAGTTCGAGGTGGACTACATCCCCGAGACC
AGCACCTCCAGAGACGTTTCCTGATGGACTGACGTCTCAAACGTGGAGAAGCAGGACATCGCCA
CCTACTACTGCGCCCTGTGGGAGG
List of human TCR Vgenes containing an Arginine corresponding
to VγR73
(IMGT numbering Vgamma9-R80)
(TRBV2)
SEQ ID NO: 12
EPEVTQTPSHQVTQMGQEVILRCVPISNHLYFYWYRQILGQKVEFLVSFYNNEISEKSEIFDDQ
FSVERPDGSNFTLKIRSTKLEDSAMYFCASSE
(TRBV6-1)
SEQ ID NO: 13
NAGVTQTPKFQVLKTGQSMTLQCAQD
MNHNSMYWYRQDPGMGLRLIYYSASEGTTDKGEVPNGYNVSRLNKREFSLRLESAAPSQTSVYF
CASSE
(TRBV6-2)
SEQ ID NO: 14
NAGVTQTPKFRVLKTGQSMTLLCAQDMNHEYMYWYRQDPGMGLRLIHYSVGEGTTAKGEVPDGY
NVSRLKKQNFLLGLESAAPSQTSVYFCASSY
(TRBV6-3)
SEQ ID NO: 15
NAGVTQTPKFRVLKTGQSMTLLCAQDMNHEYMYWYRQDPGMGLRLIHYSVGEGTTAKGEVPDGY
NVSRLKKQNFLLGLESAAPSQTSVYFCASSY
(TRBV6-4)
SEQ ID NO: 16
IAGITQAPTSQILAA GRRMTLRCTQD
MRHNAMYWYRQDLGLGLRLIHYSNTAGTTGKGEVPDGYSVSRANTDDFPLTLASAVPSQTSVYF
CASSD
(TRBV6-5)
SEQ ID NO: 17
NAGVTQTPKFQVLKTGQSMTLQCAQDMNHEYMSWYRQDPGMGLRLIHYSVGAGITDQGEVPNGY
NVSRSTTEDEPLRLLSAAPSQTSVYFCASSY
(TRBV6-6)
SEQ ID NO: 18
NAGVTQTPKFRILKIGQSMTLQCTQDMNHNYMYWYRQDPGMGLKLIYYSVGAGITDKGEVPNGY
NVSRSTTEDFPLRLELAAPSQTSVYFCASSY
(TRBV6-8)
SEQ ID NO: 19
NAGVTQTPKFHILKTGQSMTLQCAQDMNHGYMSWYRQDPGMGLRLIYYSAAAGT
TDKEVPNGYNVSRLNTEDFPLRLVSAAPSQTSVYLCASSY
(TRBV6-9)
SEQ ID NO: 20
NAGVTQTPKFHILKTGQSMTLQCAQDMNHGYLSWYRQDPGMGLRRIHYSVAAGITDKGEVPDGY
NVSRSNTEDFPLRLESAAPSQTSVYFCASSY
(TRBV7-2)
SEQ ID NO: 21
GAGVSQSPSNKVTEKGKDVELRCDPISGHTALYWYRQSLGQGLEFLIYFQGNSAPDKSGLPSDR
FSAERTGGSVSTLTIQRTQQEDSAVYLCASSL
(TRBV7-3)
SEQ ID NO: 22
GAGVSQTPSNKVTEKGKYVELRCDPISGHTALYWYRQSLGQGPEFLIYFQGTGAADDSGLPNDR
FFAVRPEGSVSTLKIQRTERGDSAVYLCASSL
(TRBV7-4)
SEQ ID NO: 23
GAGVSQSPRYKVAKRGRDVALRCDSISGHVTLYWYRQTLGQGSEVLTYSQSDAQRDKSGRPSGR
FSAERPERSVSTLKIQRTEQGDSAVYLCASSL
(TRBV7-6)
SEQ ID NO: 24
GAGVSQSPRYKVTKRGQDVALRCDPISGHVSLYWYRQALGQGPEFLTYFNYEAQQDKSGLPNDR
FSAERPEGSISTLTIQRTEQRDSAMYRCASSL
(TRBV7-7)
SEQ ID NO: 25
GAGVSQSPRYKVTKRGQDVTLRCDPISSHATLYWYQQALGQGPEFLTY
FNYEAQPDKSGLPSDRESAERPEGSISTLTIQRTEQRDSAMYRCASSL
(TRBV7-8)
SEQ ID NO: 26
GAGVSQSPRYKVAKRGQDVALRCDPISGHVSLFWYQQALGQGPEFLTYFQNEAQLDKSGLPSDR
FFAERPEGSVSTLKIQRTQQEDSAVYLCASSL
(TRBV7-9)
SEQ ID NO: 27
DTGVSQNPRHKITKRGQNVTFRCDPISEHNRLYWYRQTLGQGPEFLTYFQNEAQLEKSRLLSDR
FSAERPKGSFSTLEIQRTEQGDSAMYLCASSL
(TRBV10-1)
SEQ ID NO: 28
DAEITQSPRHKITETGRQVTLACHQTWNHNNMFWYRQDLGHGLRLIHYSYGVQDTNKGEVSDGY
SVSRSNTEDLPLTLESAASSQTSVYFCASSE
(TRBV10-2)
SEQ ID NO: 29
DAGITQSPRYKITETGRQVTLMCHQTWSHSYMFWYRQDLGHGLRLIYYSAAADITDKGEVPDGY
VVSRSKTENFPLTLESATRSQTSVYFCASSE
(TRBV10-3)
SEQ ID NO: 30
DAGITQSPRHKVTETGTPVTLRCHQTENHRYMYWYRQDPGHGLRLIHYSYGVKDTDKGEVSDGY
SVSRSKTEDFLLTLESATSSQTSVYFCAISE
(TRBV11-1)
SEQ ID NO: 31
EAEVAQSPRYKITEKSQAVAFWCDPISGHATLYWYRQILGQGPELLVQFQDESVVDDSQLPKDR
FSAERLKGVDSTLKIQPAELGDSAMYLCASSL
(TRBV11-2)
SEQ ID NO: 32
EAGVAQSPRYKIIEKRQSVAFWCNPISGHATLYWYQQILGQGPKLLIQFQNNGVVDDSQLPKDR
FSAERLKGVDSTLKIQPAKLEDSAVYLCASSL
(TRBV11-3)
SEQ ID NO: 33
EAGVVQSPRYKIIEKKQPVAFWCNPISGHNTLYWYLQNLGQGPELLIRYENEEAVDDSQLPKD
RFSAERLKGVDSTLKIQPAELGDSAVYLCASSL
(TRBV14)
SEQ ID NO: 34
EAGVTQFPSHSVIEKGQTVTLRCDPISGHDNLYWYRRVMGKEIKFLLHFVKESKQDESGMPNNR
FLAERTGGTYSTLKVQPAELEDSGVYFCASSQ
(TRBV15)
SEQ ID NO: 35
DAMVIQNPRYQVTQFGKPVTLSCSQTLNHNVMYWYQQKSSQAPKLLFHYYDKDENNEADTPDNF
QSRRPNTSFCFLDIRSPGLGDTAMYLCATSR
(TRBV19)
SEQ ID NO: 36
DGGITQSPKYLFRKEGQNVTLSCEQNLNHDAMYWYRQDPGQGLRLIYYSQIVNDFQKGDIAEGY
SVSREKKESFPLTVTSAQKNPTAFYLCASSI
(TRBV24-1)
SEQ ID NO: 37
DADVTQTPRNRITKTGKRIMLECSQTKGHDRMYWYRQDPGLGLRLIYYSFDVKDINKGEISDGY
SVSRQAQAKFSLSLESAIPNQTALYFCATSDL
(TRBV25-1)
SEQ ID NO: 38
EADIYQTPRYLVIGTGKKITLECSQTMGHDKMYWYQQDPGMELHLIHYSYGVNSTEKGDLSS
ESTVSRIRTEHFPLTLESARPSHTSQYLCASSE
(TRBV27)
SEQ ID NO: 39
EAQVTQNPRYLITVTGKKLTVTCSQNMNHEYMSWYRQDPGLGLRQIYYSMNVEVTDKGDVPEGY
KVSRKEKRNFPLILESPSPNQTSLYFCASSL
(TRBV28)
SEQ ID NO: 40
DVKVTQSSRYLVKRTGEKVFLECVQDMDHENMFWYRQDPGLGLRLIYFSYDVKMKEKGDIPEGY
SVSREKKERFSLILESASTNQTSMYLCASSL
(TRBV29-1)
SEQ ID NO: 41
SAVISQKPSRDICQRGTSLTIQCQVDSQVTMMFWYRQQPGQSLTLIATANQGSEATYESGFVID
KFPISRPNLTFSTLTVSNMSPEDSSIYLCSVE
(TRBV30)
SEQ ID NO: 42
SQTIHQWPATLVQPVGSPLSLECTVEGTSNPNLYWYRQAAGRGLQLLFYSVGIGQISSEVPQ
NLSASRPQDRQFILSSKKLLLSDSGFYLCAWS

### Overview of Vdelta2 Sequences

CDR regions are in italic font
Important residues in bold

> TRDV2*03 germline
(Used in as delta chain in GABs AJ8 and A3)
SEQ ID NO: 52
AIELVPEHQTVPVSIGVPATLRCSMKGEAIGNYYINWYRKTQGNTMTFIYREKDIYGPGFKDNF
QGDIDIAKNLAVLKILAPSERDEGSYYCACDT
> Vδ2-G31V
(Used as delta chain in GAB-AJ8s-δG31V)
SEQ ID NO: 53
AIELVPEHQTVPVSIGVPATLRCSMKGEAIVNYYINWYRKTQGNTMTFIYREKDIYGPGFKDNF
QGDIDIAKNLAVLKILAPSERDEGSYYCACDT
> Vδ2-G31M
(Used as delta chain in GAB-AJ8s-δG31M)
SEQ ID NO: 54
AIELVPEHQTVPVSIGVPATLRCSMKGEAIMNYYINWYRKTQGNTMTFIYREKDIYGPGFKDNE
QGDIDIAKNLAVLKILAPSERDEGSYYCACDT
> Vδ2-K53S
(Used as delta chain in GAB-AJ8s-δK53S and
GAB-A3s-δK53S)
SEQ ID NO: 55
AIELVPEHQTVPVSIGVPATLRCSMKGEAIGNYYINWYRKTQGNTMTFIYRESDIYGPGFKDNF
QGDIDIAKNLAVLKILAPSERDEGSYYCACDT
> Vδ2-K53A
(Used as delta chain in GAB-AJ8s-8K53A)
SEQ ID NO: 56
AIELVPEHQTVPVSIGVPATLRCSMKGEAIGNYYINWYRKTQGNTMTFIYREADIYGPGFKDNF
QGDIDIAKNLAVLKILAPSERDEGSYYCACDT
> GAB-AJ8s-Vδ2-CDR1_2acc
(KpnI + BamHII for subcloning; double BsmBI stuffer)
SEQ ID NO: 69
GGTACCCCGCCGCCACCATGGCATGCCCTGGCTTCCTGTGGGCACTTGTGATCTCCACCT
GTCTTGAATTTTCCATGGCCAGCGCTATCGAGCTGGTGCCCGAGCACCAGACCGTGCCCG
TGAGCATCGGCGTGCCCGCCACCCTGCGGTGCAGCATGAAGGGCGAGGCCATCGGCAACT
ACTACATCAACTGGTACAGAAAGACCCAGGGCAACACCATGACCTTCATCTACCGGGAGA
AGGACATCTACGGCCCTGGCTTCAAGGACAACTTCCAGGGCGACATCTGAGACGTTTCCT
AATAAAATGACGTCTCACTGGCCGTGCTGAAGATCCTGGCCCCCAGCGAGAGGGACGAGG
GCAGCTACTACTGCGCCTGCGACACTGCCGGGGGTTCTTGGGACACTCGCCAAATGTTCT
TTGGCACCGGTATAAAACTCTTTGTGGAGCCCAGAAGCCAGCCCCACACCAAGCCCAGCG
TGTTCGTGATGAAGAACGGCACCAACGTGGCCTGCCTGGTGAAAGAGTTCTACCCCAAGG
ACATCCGGATCAACCTGGTGTCCAGCAAGAAGATCACCGAGTTCGACCCCGCCATCGTGA
TCAGCCCCAGCGGCAAGTACAACGCCGTGAAGCTGGGCAAGTACGAGGACAGCAACAGCG
TGACCTGCAGCGTGCAGCACGACAACAAGACCGTGCACAGCACCGACTTCGAGGTGAAAA
CCGACTCCACCGACCACGTGAAGCCCAAAGAGACCGAGAACACCAAGCAGCCCAGCAAGA
GCTGAGGATCC

### Alignment TRDV2

```
                                                        * δ
Homo sapiens
(SEQ ID 57)
AIELVPEHQTVPVSIGVPATLRCSMKGEAIGNYYINWYRKTQGNTMTFIYREKDIYGPGFKDMFQGDIDIAKNLAVLKILAPSERDEGSYYCACDT
Homo sapiens
  (SEQ ID 58)

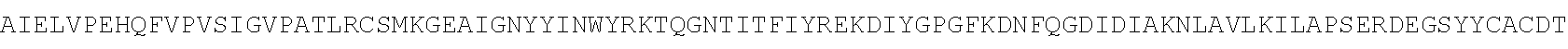

Gorilla gorilla gorilla
(SEQ ID 59)

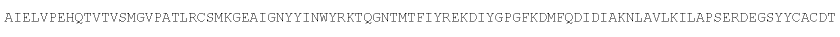

Homo sapiens
  (SEQ ID 60)

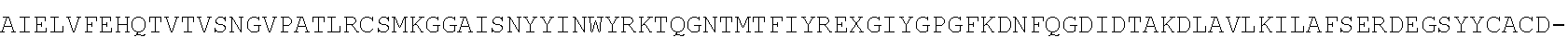

Pan troglodytes
  (SEQ ID 61)

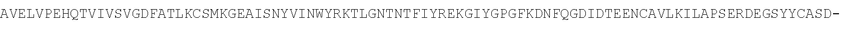

Gorilla gorilla gorilla
(SEQ ID 62)

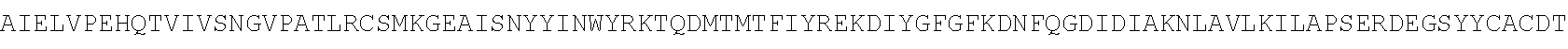

Pongo abelii
(SEQ ID 63)

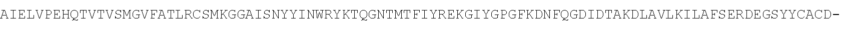

Macaca mulatta
(SEQ ID 64)

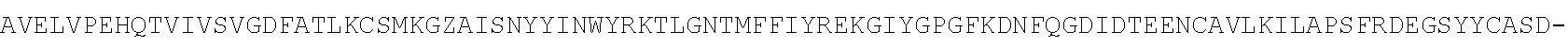

Macace mulatta
(SEQ ID 65)

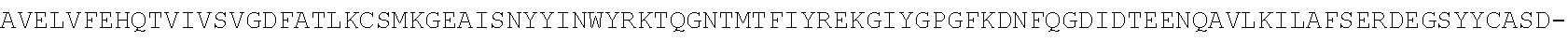

Aotus nancymaae
(SEQ ID 66)

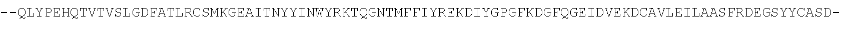

Saquinus oedipus
(SEQ ID 67)

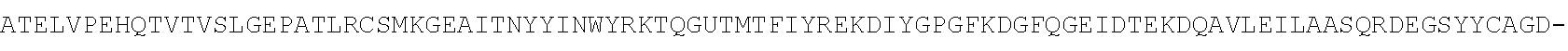

Sapa [symbol]   us apella
(SEQ ID 68)

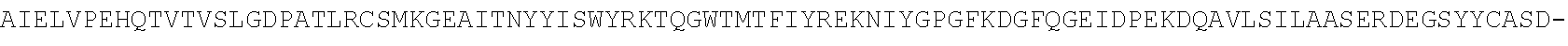

*Vd3 GLY31 Preferred substitutions: VAL, MET, ALA (hydrophobic); alternative substitutions
(slightly better to wt): ILE, LEU, SER,and [symbol]   HR
δ Vd2 LYS53 Preferred substitutions: SER, ALA
```

[symbol]

indicates data missing or illegible when filed

In case of inconsistency between the above sequences and the corresponding sequences in the sequence listing, the above sequences may be used. Alternatively, the sequences in the sequence listing may be used.

The following Examples illustrate the different embodiments of the disclosure. Unless stated otherwise all recombinant DNA techniques are carried out according to standard protocols as described in e.g., [69], [70], [71].

### EXPERIMENTAL SECTION

### Example 1

### Material and Methods

### Molecular Modeling and Affinity Prediction

The prediction of the Vγ9-BTN2A1 interaction was performed using the docking program HADDOCK using the online server (version 2.2) [28, 29]. The input model for the Vγ9 domain was derived from pdb file 1hxm, chain B residues 1-230 (see e.g., ebi.ac.uk/pdbe/entry/pdb/1hxm). For BTN2A1 a homology model was generated using the structure prediction program phyre2 [30] based on the extracellular domains of uniprot entry Q7KYR7, residues 29-248, and used as input model for HADDOCK. For each input model a list of active residues was provided, based on mutagenesis date described in literature [13, 14]. For Vγ9 the list contained residues 20, 22, 60, 70, 76, and 85, based on mutations R20A, E22A, K60A, E70A, E76A, and H85A reported by Rigau et al. [13]. For BTN2A1 selected residues were 37, 41, 96, 98, and 107 from the homology model, corresponding to mutations R65A, K79A, R124A, Y126A, and E135A reported previously [14]. The largest cluster (cluster 1) had a HADDOCK score of −103.0+/−6.5 and contained 69 models, for further analysis model 1_1 was picked. The Vγ9-BTN2A1 complex model was inspected and analyzed using Open-Source Pymol (Schrödinger, LLC). A list of Vγ9-residues, that potentially bind to BTN2A1, was made based on the following criteria: a Vγ9-residue that 1. has a Vγ9-Ca atom to BTN2A1-“any atom” distance ≤9.0 Å and 2. has a surface exposed sidechain. This list was used as input for several programs that predict changes in binding affinity between the wild type versus mutant protein. For all prediction programs the online available webservers were used with default settings. The programs used were; EvoEF [31, 32], ELASPIC2 [33, 34], and mCSM-PPI2 [35].

### Introduction of Selected Vγ9 Mutants in GAB Expression Plasmids

To introduce mutations in the Vγ9 domain of the GAB, the previously described GAB expression vector [20] was altered to facilitate rapid introduction of mutations using golden gate cloning. To this end the Vγ9 domain in the GAB expression vector was substituted by a so called “Vγ9-acceptor cassette.” In total 6 plasmids were constructed containing a specific stretch of DNA within the Vγ9 domain that could be digested with the BsmBI restriction enzyme; nucleotide sequence of the BsmBI-adapter: (A/T) GAGACGTTTCCTGATGGACTGACGTCTCA (SEQ ID:45, 46). Sequences of the 6 different Vγ9-acceptor cassettes are listed in appendix 1.

For the selected mutations DNA oligo pairs were designed for the corresponding acceptor to reintroduce the Vγ9-residues including a single point mutation. The DNA oligos were ordered at Integrated DNA Technologies Europe. Both the sense and antisense oligos had a 4 nucleotide 5′ extension compatible with the overhangs left after BsmBI digestion. After annealing of the oligo pairs, these double stand DNA fragments, with compatible overhangs, were ligated in the BsmBI-digested Vγ9-acceptor vector using T4 ligase and after a short (5′-15′) incubation at room temperature, the ligation reaction mix was used to transform E. coli TOP10 cells using a standard heat shock protocol. The transformed bacteria were plated on ampicillin containing LB-ager plates for selection. Single colonies were used to inoculate liquid LB cultures for plasmid preparation using the NucleoSpin Plasmid EasyPure kit from Macherey-Nagel. The purified plasmids were send for Sanger sequencing to confirm the successful introduction of the point mutations.

### Expression of GABs in 293 F Cells

293 F cells were cultured in Gibco Freestyle Expression medium and transfected with DNA: polyethylenimine (PEI) complexes for the expression of GABs. 25 kDa linear PEI (Polysciences, Germany) was added in a 3-to-1 ratio to DNA in Freestyle medium. Per million cells, 1.25 μg DNA and 3.75 g PEI were premixed in 1/10 or 1/30 of the culture volume and after a brief vortex incubated for 10′-15′ at room temperature. The DNA:PEI mix was added dropwise to the cultures with a cell density of 10\hat{ }6 cells/ml. The cultures were incubated on a shaking platform at 37° C. with 8% CO₂ for 6 days. At day 6 the expression medium was harvested after centrifugation at 3000×g for 10′.

### Western Blot Analysis

293 F expression media was mixed with 4× leammli non-reducing sample buffer (Biorad) and incubated for 5′ at 95° C. Samples were loaded on a 4-20% Miniprotean TGX gel (Biorad) after electrophoresis the gel was put on a nitrocellulose membrane (Trans-blot turbo; Biorad) and the proteins were transferred to the membrane. The membrane was incubated with 5% milk powder solution in PBS for 45′ at room temperature. The membrane was incubated with 5 ml of PBS-tween containing 1 μl anti 6×His (BD Pharmingen) and 0.75 μl Goat-anti-mouse HRP (southern biotech) for 30′ at room temperature. The blot was washed extensively with PBS-T and incubated for 30″ with ECL reagent (Cytiva Amersham) and imaged using a ChemiDoc MP Imaging System (Biorad).

### Purification of GABs

GABs were purified from the expression medium using a 2 step purification protocol as described before [20]. In short, after concentration and diafiltration of the expression media using a Vivaflow 200 cassette (10 kDa MWCO, Sartorius, Germany), it was loaded on a 1 ml HisTrap column (Cytivia) and the His tag containing GAB was eluted using a linear imidazole gradient. Fractions containing GAB were buffer exchanged to a low ionic strength buffer and loaded on a 1 ml HiTrap Q column (Cytivia) and eluted using a linear NaCl gradient. Fractions containing GAB were concentrated and stored in 20 μg aliquots at −80° C. until further use. All buffers were at pH 8.0.

### Cells and Cell Lines

Buffy coats obtained from Sanquin Blood Bank (Amsterdam, The Netherlands) were used to isolate PBMCs using Ficoll-Paque Plus (Cytivia). T cells were expanded from PBMCs using CD3/CD28 dynabeads (Thermo Fisher Scientific) in RPMI1640 medium (Gibco) containing 2.5% heat inactivated pooled human serum (Sanquin Blood Bank, The Netherlands), 1% Pen/Strep (Invitrogen) and 1.7×10³ IU/ml of rhIL-7 and 1.5×10² IU/ml rhIL-15 (both Milteny Biotec, Germany). 48-72 hours before T cell assays the media was replaced with media without cytokines.

The adherent cell lines, SCC9(-lucGFP) and MZ1851RC(-lucGFP), were cultured in DMEM medium (Gibco) supplemented with 10% FCS (Bodinco, The Netherlands) and pen/strep (Invitrogen). RMPI8226(-lucGFP) was cultured in RPMI1640 medium supplemented with 10% FCS and 1% Pen/Strep.

TEGs were generated as reported before [19]. In short T cells were transduced with retroviral particles containing DNA encoding γTCR chain, δTCR chain, and antibiotic resistance genes. After the antibiotic selection the TEGs were expanded using a Rapid Expansion Protocol before functional testing.

### IFNγ Release Assay Using GAB Expression Medium

293 F expression medium containing GAB (mutants) was serial diluted in complete RPMI1640 medium (3-fold dilution series), 50 μl was transferred to a 96 well U-bottom plate. Rested T cells were resuspended in complete RPMI1640 at a concentration of 10\hat{ }6 cells/ml and 50 μl was added to the GABs in the 96 well U-bottom plate. Tumor cell lines were resuspended in complete RPMI1640 medium supplemented with 120 μM pamidronate at a concentration of 10\hat{ }6 cells/ml for SCC9 or 5 10≡cells/ml for MZ1851RC and 50 μl of a tumor cell line suspension was added to the wells containing GAB and T cells. The co-cultures were incubated for 20 h at 37° C. 5% CO₂, cells were pelleted using centrifugation, and the IFNγ concentrations was determined using the Human IFN gamma Uncoated ELISA Kit (Invitrogen). Each plate contained next to the GAB-AJ8 mutants expression medium dilution, also expression medium of GAB-AJ8s and GAB-LM1 as controls. The IFNγ concentration of each GAB-AJ8 mutant was normalized to the corresponding dilution of GAB-AJ8s.

### IFNγ Release Assay Using Purified GAB

5 10\hat{ }4 effector cells and 5 10\hat{ }4 target cells or 2.5 10\hat{ }4 MZ1851RC cells, were incubated together, with or without GAB (different concentrations, as indicated) for 20 hours at 37° C. 5% CO₂. To increase intracellular phosphoantigen levels pamidronate was added to the co-culture at concentrations indicated in the figure legends. The supernatant was harvested after 20 hours, and the level of IFNγ was determined using the IFN gamma Human Uncoated ELISA Kit (Invitrogen).

### Luciferase-Based Killing Assay

10\hat{ }4 target cells stably expressing luciferase were incubated with T cells in a 1:5 target/effector cell ratio, with different GAB concentrations (as indicated) in the absence or presence pamidronate (calbiochem, United States). Or 10\hat{ }4 RPMI8226-lucGFP cells were incubated with TEGs at different target/effector cell ratio in the absence or presence pamidronate After 20 hours, beetle luciferin (Promega, United States) was added to the wells (125 μg/ml) and bioluminescence was measured on SoftMax Pro plate reader. The signal in treatment wells was normalized to the signal measured for targets and T cells only, which was assumed to represent 100% living cells.

### Results

Prediction Vγ9 Mutations that Increase the Affinity for BTN2A1

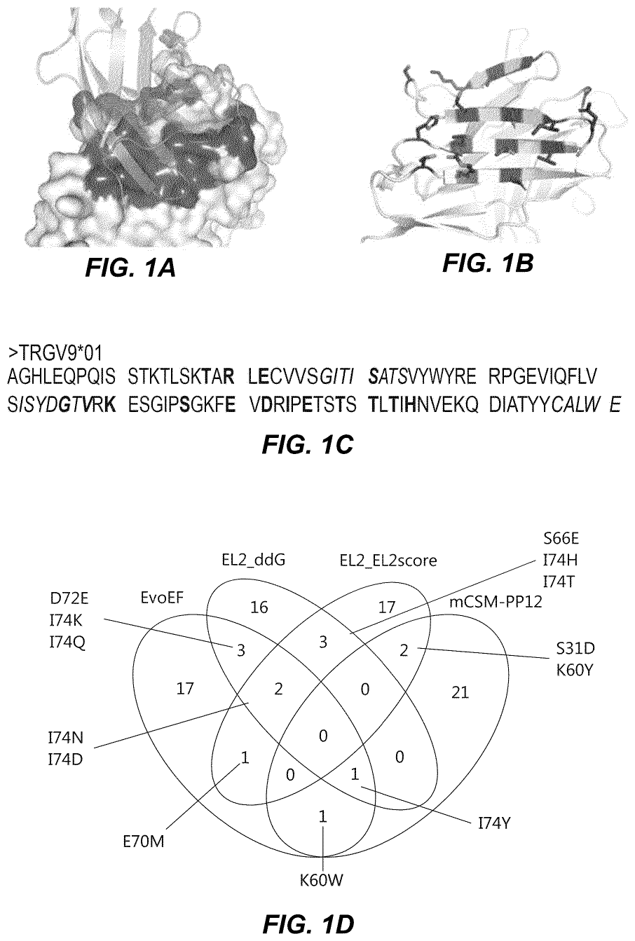

The first step of the approach to improve the affinity of the Vγ9 domain for BTN2A1 was to generate a molecular interaction model of the Vγ9 domain-BTN2A1 complex. For this, the mutations that affect the affinity of these proteins were used, which have been reported by Rigau et al. and Karunakaran et al. [13, 14], as input for the docking program HADDOCK [28, 29]. As expected, all input residues were located in the interface of the predicted complex and the overall domain-domain orientations resembles the model that Karunakaran et al. have shown in their report [14]. Sixteen residues in the Vγ9 domain were identified that fulfilled the two criteria for further investigation, these residues were in close proximity to BTN2A1, Vγ9-Cα to BTN2A1 distance ≤9.0 Å, and had a surface exposed sidechain (FIG. 1A). As mutating and testing these 16 residues to all other amino acids, resulting in 304 possibilities, using experimental methods would be a daunting task, computational affinity predictors narrowing down the number of mutations to test was explored. Three different online available affinity predictors, EvoEF, ELASPIC2, and mCSM-PPI2, were used to predict the effect of single mutations in any of the 16 residues; for ELASPIC2 both the ddG as well as EL2 score were used in further analysis. The prediction of the most favorable mutations was found to differ substantially between the affinity predictors. To limit the analysis to the most favorable mutations, the Top25 of each prediction output were used for further comparison. Within this set of mutations, 15 out of the 16 interface residues were present, only T79 was not in any of the Top25s with any mutation, likely due to the large distance, 9.0 Å, to BTN2A1. Only 13 mutations were shared in the Top25 of at least 2 prediction outputs, and 3 of these mutations were in the Top25 of 3 prediction outputs (FIG. 1), resulting in 84 unique mutations for further testing. The limited overlap in the predicted affinity enhancing mutations is in part due to the input model, which was not experimentally determined and thus has a relatively high degree of uncertainty. The quality of the input model is very important for the performance of affinity predictors [36]. A secondary reason for the difference in the Top25 mutants between the different predictors is the differences in underlying algorithms of the predictors.

### Mutation Vγ9_{R73Y} Increases GAB Production Yield Twofold

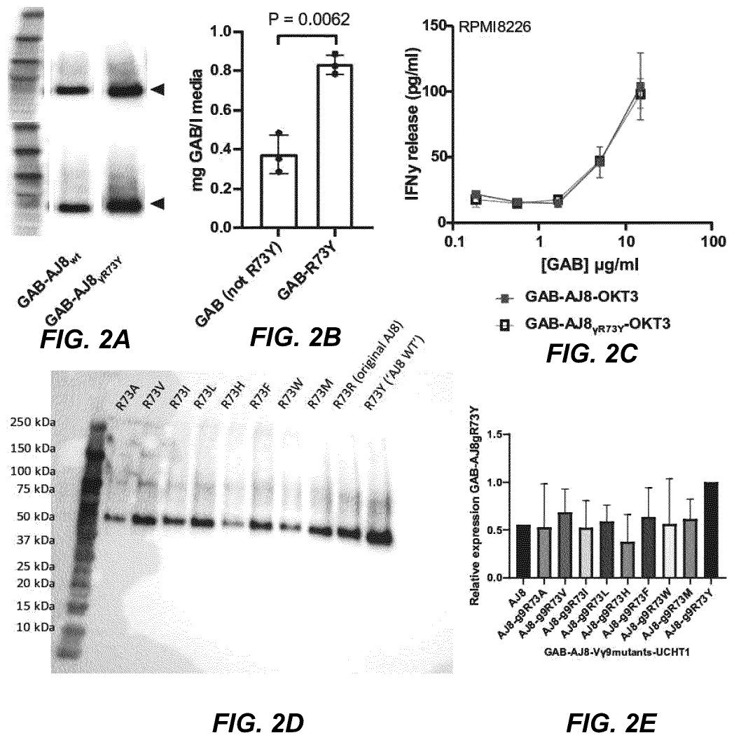

The primary goal was increasing the potency of the Vγ9 domain in Vγ9Vδ2 TCR based therapeutics, but mutations that increase the production yield of soluble Vγ9Vδ2 TCRs was also interesting, as poor protein yield could be a limiting step for translating soluble therapeutic formats, like GABs, into the clinic. While inspecting the modeled Vγ9-BTN2A1 interface for Vγ9 residues involved in the interaction with BTN2A1, an arginine was found that had its charged side chain inserted in to the hydrophobic core of the Vγ9 domain, Vγ9_{R73}. Although such buried charged residues can be present in proteins, in many cases they either form a salt bridge with a residue of opposite charge or have important functions in the protein function [37]. In general, buried ionizable amino acids are tolerated, but it was considered that they may tend to reduce the stability of the protein. Therefore, the arginine was substituted for a bulky tyrosine and it was noticed that the expression was increased in initial test expressions that were analyzed by western blot (FIG. 2A). As a change in western blot intensity of the GAB might not translate into increased protein yields after purification, a side-by-side expression and purification analysis of GABs was performed with or without the Vγ9_{R73Y}. This mutation significantly increased the GAB yield after purification twofold (FIG. 2B).

As the Vγ9_{R73Y} mutation is located in/near the BTN2A1 binding site, the Vγ9_{R73Y} mutation could affect GAB activity. As increased GAB yields are only useful when there is no loss in activity, T cells activation potential of GAB-AJ8_{wt} and GAB-AJ8_{γR73Y} was compared using an IFNγ release assay targeting the RPMI8226 cell line. No differences in activation were observed between GAB-AJ8_{wt} and GAB-AJ8_{γR73Y}, demonstrating that the Vγ9_{R73Y} mutation can be used without a negative effect on GAB potency (FIG. 2C).

In order to test whether other hydrophobic residues at position Vγ9-R73 were able to induce an increase in expression as seen for Vγ9_{R73Y}, Vγ9-R73 was substituted for the following hydrophobic residues, alanine, valine, isoleucine, leucine, histidine, phenylalanine, tryptophan, and methionine, in GAB-AJ8 and assessed their expression level in a small scale HEK293F culture using Western blot analysis (FIG. 2D). Although expression levels were reasonably good, none of the other substitutions for Vγ9-R73 resulted in higher expression levels compared to Vγ9_{R73Y} (FIG. 2E).

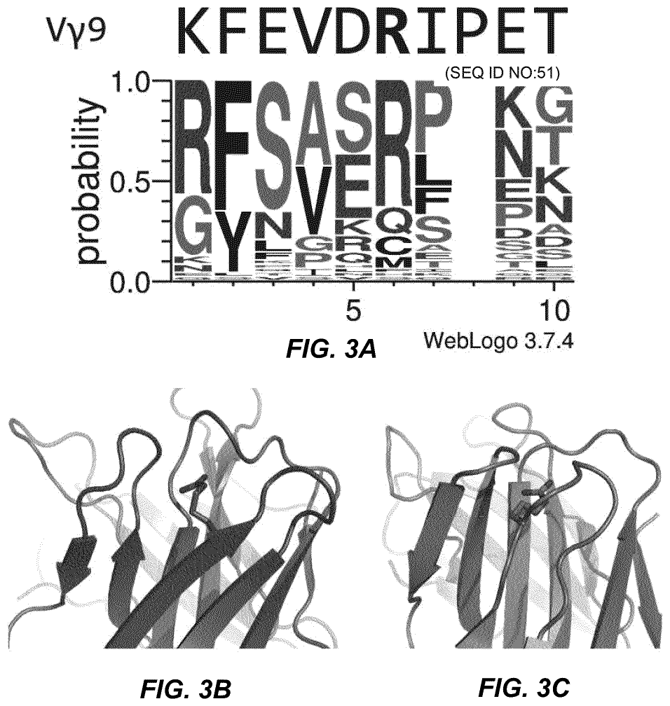

The R73 in Vγ9 is not present at equivalent positions in other human Vγ genes, but when the human Vβ genes deposited in IMGT[40] were analyzed, it was found that there is an arginine present at this position in over half of the functional Vβ genes (Table 1, FIG. 3A). A structural comparison between TRGV9 and TRBV6 containing TCRs showed that in both the γ as β chain the arginine was buried in the hydrophobic core of the variable domain (FIGS. 3B and 3C). Mutating this arginine in the Vβ genes to a tyrosine will most likely lead to a more stable β chain as well.

TABLE 1
Comparison between y9 and ß chains shows that in both the y as β chain the arginine is buried in the hydrophobic core of the variable domain
(FIGS. 3B and 3C). Mutating this arginine in the β chain to e.g., a tyrosine will lead to a more stable ß chain.

| Human | |
|---|---|
| Vgene | Sequence alignment according to IMGT |
| | [symbol] |
| TRGV9 | ACHLEQPQISSTKTL SKTARLECVVS GITI....SATS VYWYRERP GEVIQFLVS ISYD...GTV RKESGIPSG KFEVDRIPET STSTLTIHNVEK QDIATYYC ALWEV |
| TRBV2 | EPEVTQTPSHQVTQM GQEVILRCVPI SNH.......LY FYWYRQIL GQKVEFLVS FYN....NEI SEKSEIFDQ QFSVERP.DG SNFTLKIRSTKL EDSAMYFC ASSE |
| TRBV6-1 | NAGVTQTPKFQVLKT GQSMTLQCAQQ MNH.......NS MYWYRQDP GMGLRLIYY SAS....EGT TQKSEVP.N GYNVSRL.NK REFSLRLESAAP SQTSVYFC ASSE |
| TRBV6-2 | NAGVTQTPKFRVLKT GQSMTLLCAQQ MNH.......EY MYWYRQDP GMGLRLIHY SVG....EGT TAKGEVP.D GYNVSRL.KK QNFLLGLESAAP SQTSVYFC ASSY |
| TRBV6-3 | NAGVTQTPKFRVLKT GQSMTLLCAQD MNH.......EY MYWYRQDP GMGLRLIHY SVG....EGT TAKGEVP.D GYNVSRL.KK QNFLLGLESAAP SQTSVYFC ASSY |
| TRBV6-4 | IAGITQAPTSQILAA GRRMTLRCTQD MRH.......NA MYWYRQDŁ GLGLRLIHY SNT....AGT TGKGEVP.D GYSVSRA.NT DDFPLTLASAVP SQTSVYFC ASSD |
| TRBV6-5 | NAGYTQTPKFQVLKT GQSMTLQCAQD MNH.......EY MSWYRQDP GMGLRLIHY SVG....AGI TDQSEVP.N GYNVSRS.TT EDFPLRLLSAAP SQTSVYFC ASSY |
| TRBV6-6 | NAGYTQTPKFRILKI GQSMTLQCTQQ MNH.......NY MYWYRQDP GMGLKLIYY SVG....AGI TDKGEVP.N GYNVSRS.TT EDFPLRLELAAP SQTSVYFC ASSY |
| TRBV6-8 | NAGYTQTPKFHILKT GQSMTLQCAQQ MNH.......GY MSWYRQDP GMGLRLIYY SAA....AGT TDK.EVP.N GYNVSRL.NT EDFPLALVSAAP SQTSVYLC ASSY |
| TRBV6-9 | NAGYTQTPKFHILKT GQSMTLQCAQQ MNH.......GY LSWYRQDP GMGLRRIHY SVA....AGI TDKGEVP.D GYNVSRS.NT EDFPLALESAAP SQTSVYFC ASSY |
| TRBV7-2 | GAGVSQSPSNKVTEK GKDVELRCDPI SGH.......TA LYWYRQSL GQGLEFLIY FQG....NSA PDKSGLPSD RFSAERT.GG SVSTLTIQRTQQ EDSAVYLC ASSL |
| TRBV7-3 | GAGVSQTPSNKVTEK GKYVELRCDPI SGH.......TA LYWYRQSL GQGPEFLIY FQG....TGA ADDSGLPND RFFAVRP.EG SVSTLKIQRTER GDSAVYLC ASSL |
| TRBV7-4 | GAGVSQSPRYKVAKR GRDVALRCDSI SGH.......VT LYWYRQTL GQGSEVLTY SQS....DAQ RDKSGRPSG AFSAERP.ER SVSTLKIQRTEQ GDSAVYLC ASSL |
| TRBV7-6 | GAGVSQSPRYKVTKR GQDVALRCDPI SGH.......VS LYWYRQAL GQGPEFLTY FNY....EAQ QDKSGLPND RFSAERP.EG SISTLTIQRTEQ RDSAMYRC ASSL |
| TRBV7-7 | SAGVSQSPRYKVTKR GQQVTLRCQPI SSH.......AT LYWYQQAL GQGPEFLTY FNY....EAQ PDKSGLPSD RFSAERP.EG SISTLTIQRTEQ RDSAMYRC ASSL |
| TRBV7-8 | GAGVSQSPRYKVAKR GçDVALRCQPI SGH.......VS LFWYQQAL GQGPEFLTY FQN....EAQ LDKSGLPSD RFFAERP.EG SVSTLKIQRTQQ EDSAVYLC ASSL |
| TRBV7-9 | DTGVSQNPRHKITKR GQNVTFRCDPI SEH.......NR LYWYRQTL GQGPEFLTY FQN....EAQ LEKSRLLSD RFSAERP.KG SFSTLEIQRTEQ GDSAMYLC ASSL |
| TRBV10-1 | DAEITQSPRHKITET GRQVTLACHQT WNH.......NN MFWYRQDL GHGLRLIHY SYG....VQQ TNKGEVS.D GYSVSRS.NT EDLPLTLESAAS SQTSVYFC ASSE |
| TRBV10-2 | DAGITQSPRYKITET GRQVTLMCHQT WSH.......SY MFWYRQDL GHGLRLIYY SAA....ADI TDKGEVP.D GYVVSRS.KT ENFPLTLESATR SQTSVYFC ASSE |
| TRBV10-3 | DAGITQSPRHKVTET GTPVTLRCHQT ENH.......RY MYWYRQDP GHGLRLIHY SYG....VKD TDKGEVS.D GYSVSRS.KT EDFLLTLESATS SQTSVYFC AISE |
| TRBV11-1 | EAEVAQSPRYKITEK SQAVAFWCDPI SGH.......AT LYWYRQIL GQGPELLVQ FQD....ESV VDDSQLPKD RFSAERL.KG VDSTLKIQPAEL GDSAMYLC ASSL |
| TRBV11-2 | EAGVAQSPRYKIIEK RQSVAFWCNPI SGH.......AT LYWYQQIL GQGPKLLIQ FQN....NGV VDDSQLPKD RFSAERL.KG VQSTLKIQPAKL EDSAVYLC ASSL |
| FRBV11-3 | EAGVVQSPRYKIIEK KQPVAFWQNPI SGH.......NT LYWYLQNL GQGPELLIR YEN....EEA VDDSQLPKD RFSAERL.KG VDSTLKIQPAEL GDSAVYLC ASSL |
| TRBV14 | EAGVTQFPSHSVIEK GQTVTLRCQPI SGH.......QN LYWYRRVM GKEIKFLLH FVK....ESK QDESGMPNN RFLAERT.GG TYSTLKVQPAEL EDSGVYFC ASSQ |
| TRBV15 | DAMVIQNPRYQVTQF SKPVTLSCSQT LNH.......NV MYWYQQKS SQAPKLLFH YYD....KDF NNEADTP.D NFQSRRP.NT SFCFLDIRSPGL GDTAMYLC ATSR |
| TRBV19 | DQGITQSPKYLFRKE GQNVTLSCEQN LNH.......DA MYWYRQDP GQGLALIYY SQI....VND FQKSDIA.E GYSVSRE.KK ESFPLTVTSAQK NPTAFYLC ASSI |
| TRBV24-1 | DADVTQTPRNRITKT GKRIMLECSQT KGH.......DR MYWYRQDP GLGLRLIYY SFD....VKD INKGEIS.D GYSVSRQ.AQ AKFSLSLESAIP NQTALYFC ATSDL |
| TRBV25-1 | EADIYQTPRYLVIGT GKKITLECSQT MGH.......DK MYWYQQDP GMELHLIHY SYG....VNS TEKSDLS.S ESTVSRI.RT EHFPLTLESARP SHTSQYLC ASSE |
| TRBV27 | EAQVTQNPRYLITVT GKKLTVTCSQN MNH.......EY MSWYRQDP GLGLRQIYY SMN....VEV TDKGQVP.E GYKVSRK.EK RNFPLILESPSP NQTSLYFC ASSL |
| TRBV2S | DVKVTQSSRYLVKRT GEKVFLECVQD MDH.......EN MFWYRQDP GLGLRLIYF SYD....VKM KEKGDIP.E GYSVSRE.KK ERFSLILESAST NQTSMYLC ASSL |
| TRBV29-1 | SAVISQKPSRDICQR GTSLTIQCQVD SQV.......TM MFWYRQQP GQSLTLIAT ANQG...SEA TYESGFVID KFPISRP.NL TESTLTVSNMSP EDSSIYLC SVE |
| TRBV30 | SQTIHQWPATLVQPV GSPLSLECTVE GTS......NFN LYWYRQAA GRGLQLLFY SVG.....IG QISSEVP.Q NLSASRP.QD RQFILSSKKLLL SDSGFYLC ANS |
| | [symbol] |

### Limited Number of Predicted Vγ9 Mutations Enhance T Cell Activation Compared to Vγ9s

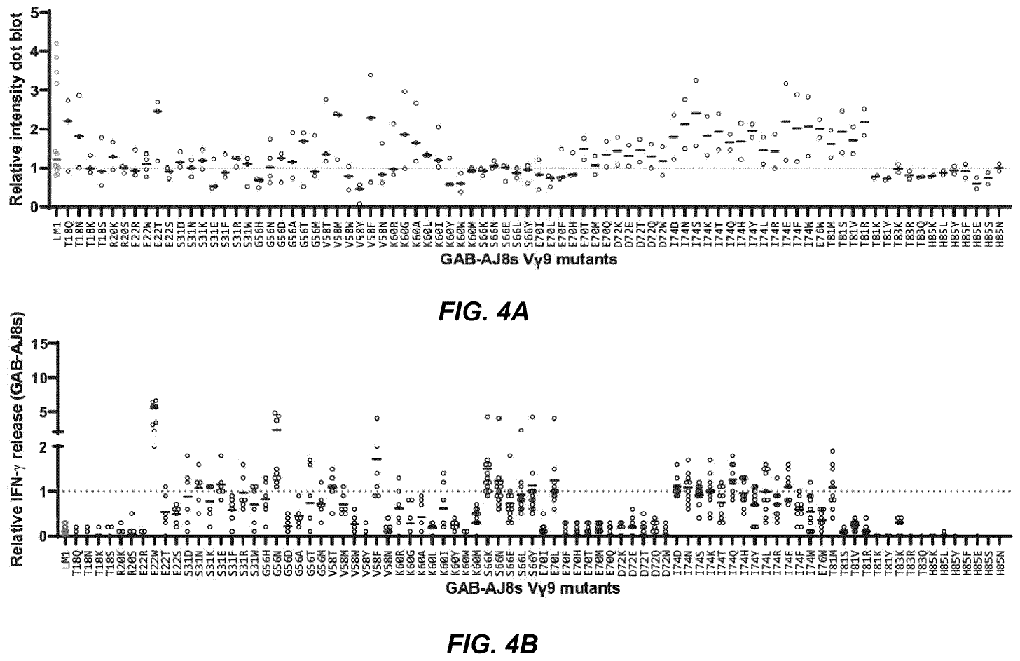

To assess the effect of the predicted mutations on the activity of the Vγ9Vδ2 TCR, the recently developed GAB [20] was used. All 84 unique mutations from the prediction output were introduced, as an additional mutation next to Vγ9_{R73Y}, in the Vγ9-chain of TCR AJ8 using the golden gate assembly strategy [41]. GABs containing the expression enhancing mutation Vγ9_{R73Y} will be indicated with an addition ‘s’ following the TCR name, like this GAB-AJ8s. After sequence verification, the plasmids were used to transiently transfect HEK293F cells for GAB production. Five days after transfection the expression media was harvested and GAB expression levels were determined using dot blot. All mutants were expressed to comparable levels as wild type GAB, but the dot blot signal varied amongst replicates, making in depth analysis of the expression levels not warranted (FIG. 4A).

The GAB containing expression media was used to test the T cell activation potential of the GAB mutants in a co-culture of T cells with tumor cells, either MZ1851RC or SCC9. To boost intracellular phosphoantigen levels in the tumor cells, 30 μM of pamidronate was included in the co-culture. As marker for T cell activation, IFNγ release was used, which was measured using an IFNγ ELISA. Next to the AJ8 mutants and AJ8s GABs [20], a poorly activating GAB was used as a negative control, based on Vγ9Vδ2 TCR LM1 [19]. To be able to compare the results between the different assays, the IFNγ release was normalized to GAB-AJ8s condition on the same assay plate. Almost half of the mutants induced no or low IFNγ release by the T cells, comparable to GAB-LM1s. As expected, mutations introduced in Vγ9 residues that had been identified by Rigau et al. to be crucial for BTN2A1 binding, such as R20, E70, and H85 [13], also abrogated T cell activation in this assay, except for mutation E70L, which had comparable activity to GAB-AJ8s. Next to these already reported residues, additional Vγ9 residues were found in the screen for which mutagenesis resulted in strongly reduced T cell activation potential, for most or all substitutions tested. These newly identified Vγ9 residues, that were important for the interaction with BTN2A1, were in numerical order: T18, E22, D72, T81, T83. For other Vγ9 residues tested in our screen, like S31, S66, and 174, mutagenesis did not result in a decrease in T cell activation potential of GAB for any of the introduced mutations, making it not likely that these residues are essential for the interaction with BTN2A1 (FIG. 4B).

Out of the 84 mutants tested in GAB screening, only a few induced more T cell activation compared to GAB-AJ8s. One of these mutants, Vγ9_{E22W}, showed the most potent induction of T cell activation in the IFNγ release assay, with a mean fold increase of 5.6. Some other mutations induced fractional higher T cell activation, between 1.2- and 2.3-fold increase, with a greater inter assay variability, like G56N, V58F, S66K, S66N, E70L, and I74Q (FIG. 4B).

Mutation Vγ9_{E22W} increases GAB potency 10-fold

In the screening method, GAB, containing expression media, was used to identify potential activity enhancing mutations. One limitation of the screening method was that it did not correct for the difference in GAB concentration, thus observed differences in cytokine secretion could merrily reflect the differences in protein yield. For a more reliable comparison between GAB-AJ8s and the Vγ9-mutations, selected Vγ9-mutants were expressed and purified, with a focus on mutants, which showed during screening enhanced activity, namely E22W, G56N, V58F, S66K, E70L, and I74Q. This set of purified GABs was used in T cell activation assays and titrated GAB mutants to allow a more thorough assessment of differences in potency.

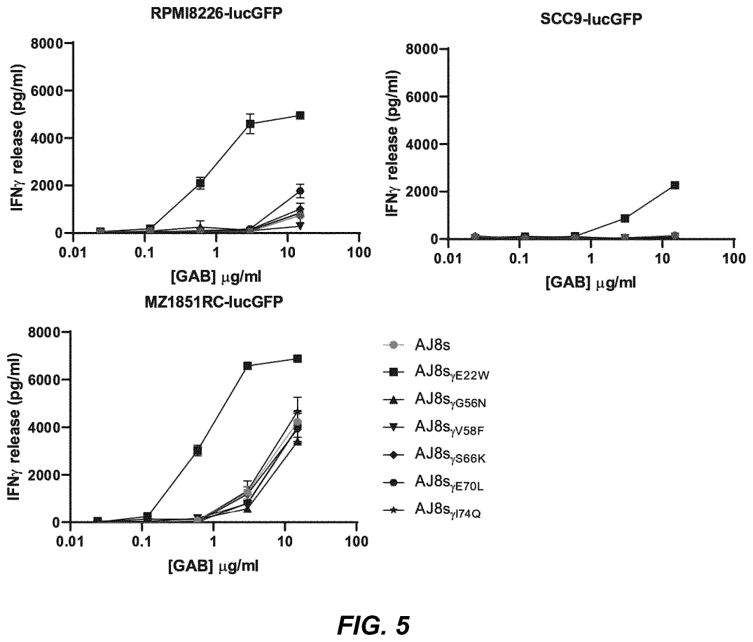

First, the IFNγ release of T cells co-cultured with tumor cell lines and GABs was assessed in presence of 30 μM pamidronate. As seen in the initial screening, GAB-AJ8_{γE22W} induced higher levels of IFNγ compared to GAB-AJ8s and was also able to induce T cell activation at lower GAB concentration for all 3 tested cell lines. The other GAB-Vγ9-mutants had comparable activity to GAB-AJ8s (FIG. 5).

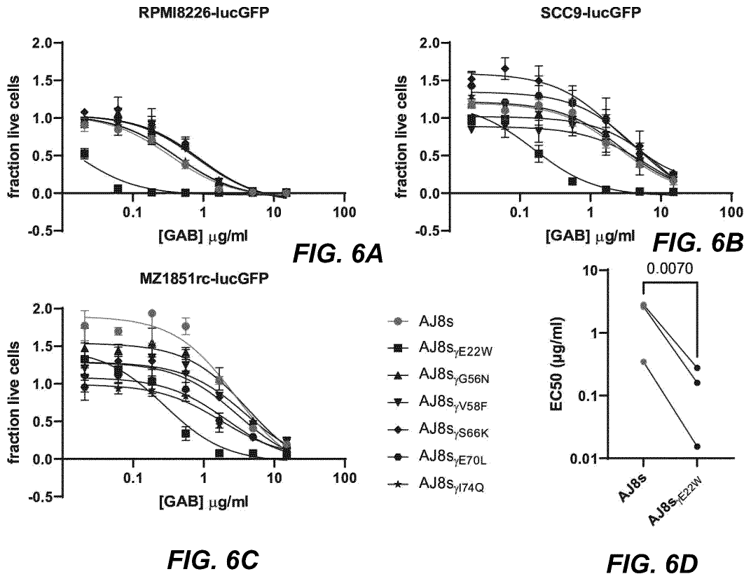

Next to IFNγ release, the effects of the Vγ9-mutants on T cell induced tumor cytotoxicity were also interesting. Luciferase expressing tumor cell lines were co-cultured with T cells, GABs and 30 μM pamidronate and after 20 hours the fraction of viable cells was determined using luciferase activity as read out. Again GAB-AJ8_{γE22W} stood out, increasing the activity >10 fold, for all 3 tumor cell lines (FIGS. 6A-6C). While for the other Vγ9-mutations there was no consistent enhancement in potency across the different tumor cell lines (FIGS. 6A-6C), which was more apparent when the EC50 was calculated. Analysis of the differences in EC50 values between GAB-AJ8s and GAB-AJ8_{γE22W} showed a significant increase in potency for GAB-AJ8_{γE22W}, with a mean fold difference of 15.5 (95% CI; 5.8-41.9) (FIG. 6D).

### Mutation Vγ9_{E22W} Decreases the Dependency on Pamidronate for Tumor Cell Recognition

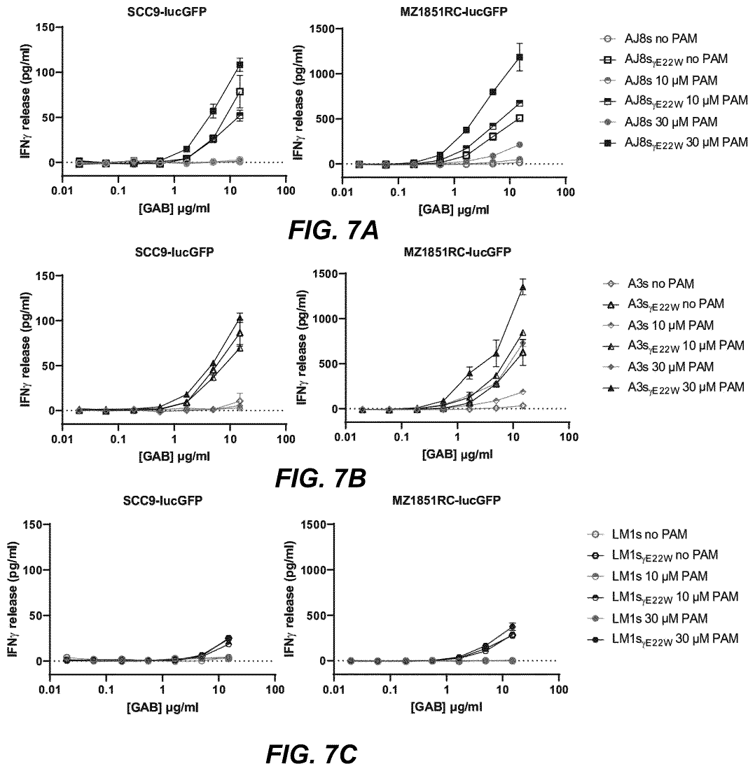

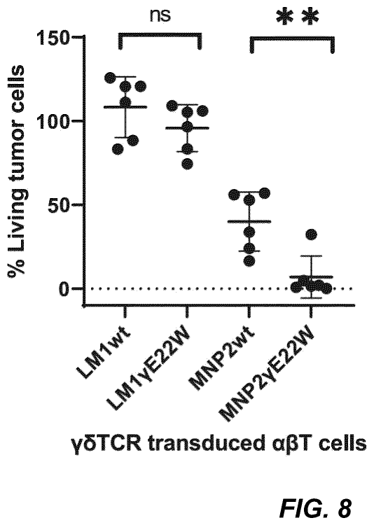

One of the major obstacles in Vγ9Vδ2 TCR based therapies is the low levels of phosphoantigens in tumor cells, leading to a suboptimal activation of Vγ9Vδ2 T cells or Vγ9Vδ2-TCR based therapies. Many of the current strategies require the co-administration of aminobisphosphonates, such as pamidronate or zoledronate, to increase the intracellular levels of phosphoantigens[8]. In an earlier report, differences in pamidronate dependency of GAB was observed between tumor cell lines, SCC9 required a high concentration of pamidronate to be targeted by GABs, while MZ1851RC was already recognized without the addition of pamidronate [20]. Here, the influence of pamidronate on the activation potential of GAB-AJ8s_{γE22W} was tested by assessing the T cell activation without pamidronate and in presence of 10 and 30 μM pamidronate. In line with earlier findings, GAB-AJ8s could not induce any T cell activation in combination with the highly pamidronate dependent cell line SCC9, however in all 3 pamidronate conditions GAB-AJ8s_{γE22W} was able to induce T cell activation, with no large differences in IFNγ release between the pamidronate conditions (FIG. 7A). In contrast to previous results, no T cell activation is induced by GAB-AJ8s without the addition of pamidronate using MZ1851RC as target cell line, while at 30 μM pamidronate GAB-AJ8s did induce T cell activation. As seen for SCC9, GAB-AJ8s_{γE22W} also induced T cell activation at all 3 pamidronate conditions using MZ1851RC. For this target cell line, a larger effect of the pamidronate concentration was not seen, with over a 2-fold increase in IFNγ at the highest GAB concentration (15 μg/ml), between the no pamidronate and 30 μM pamidronate condition (FIG. 7A).

Previously, it has been shown that the CDR3δ can have a substantial influence on the activation potential of GABs. GAB-AJ8s has intermediate potency, while two other reported Vγ9Vδ2TCR used in the GAB format, GAB-C15 and GAB-A3, showed high potency[20]. In order to test if the Vγ9_{E22W} mutation also enhanced the potency of GAB-A3, produced GAB-A3s_{γE22W} was produced and its potency was compared to GAB-A3s in T cell activation assay. Again, SCC9 and MZ1851RC were used as target cell lines and incubated them with T cells, three concentrations pamidronate and a dilution series of the GABs. Similar to what was observed for GAB-AJ8, GAB-A3s did not induce any T cell activation in combination with SCC9, while GAB-A3s_{γE22W} did induce T cell activation (FIG. 7B). However, no clear differences in activation potential between GAB-AJ8s_{γE22W} and GAB-A3s_{γE22W} could be observed (FIGS. 7A, 7B). When using MZ1851RC as target cell line, GAB-A3s did induce T cell activation in the presence of 10 and 30 μM pamidronate, but GAB-A3s_{γE22W} induced higher levels of IFNγ release at each pamidronate concentration (FIG. 7B).

The limited difference in T cell activation potential between GAB-AJ8s_{γE22W} and GAB-A3s_{γE22W} raises the question whether the CDR3δ sequence is important at all. To investigate this, the poorly functional Vγ9Vδ2 TCR LM1[19] was used. As described above, the T cell activation potential of GAB-LM1s and GAB-LM1s_{γE22W} was compared using the tumor cell lines SCC9 and MZ1851RC. In line with earlier observations, GAB-LM1s does not induce IFNγ release by T cells in any of the conditions (FIG. 7C)[20]. The introduction of the Vγ9_{E22W} mutation in GAB-LM1s led to a slight increase in INFγ release by T cells for both tumor cell lines, SCC9 and MZ1851RC, irrespective of the pamidronate concentration (FIG. 7C). However, there is a clear difference in T cell activation potential between GAB-LM1s_{γE22W} and the more potent GAB-AJ8s_{γE22W} and GAB-A3s_{γE22W}, confirming that GAB potency was still dependent on the CDR3δ composition after the introduction of the Vγ9_{E22W} mutation, although less pronounced compared GABs consisting of a wild type V79 domain.

Mutation Vγ9_{E22W} Increases Potency of αβT Cells Transduced with Vγ9Vδ2TCR

To explore if the Vγ9_{E22W} mutation would also increase the potency Vγ9Vδ2TCR transduced αβT cells [19, 21], αβT cells expressing Vγ9Vδ2TCRs were generated with or without the Vγ9_{E22W} mutation using retroviral transduction. Four different Vγ9Vδ2TCRs were transduced; the poorly activating Vγ9Vδ2TCR LM1s (wt and Vγ9_{E22W}) and the functional Vγ9Vδ2TCR MNP2 (wt and Vγ9_{E22W}). These transduced T cells were assessed for their killing potential using luciferase transduced RPMI8226 cells. As expected Vγ9Vδ2TCR LM1wt did not induce any killing, while the functional Vγ9Vδ2TCR MNP2 wt killed around 60% of the RPMI8226 cells. Comparable to the GAB induced activation, Vγ9Vδ2TCR LM1_{γE22W} showed little activity, while Vγ9Vδ2TCR MNP2_{γE22W} increased the killing potential of the transduced T cells significantly, resulting in over 90% cell dead of RPMI8226 cells (FIG. 8).

### Screening for Additional Enhancing Mutations in Important Residues

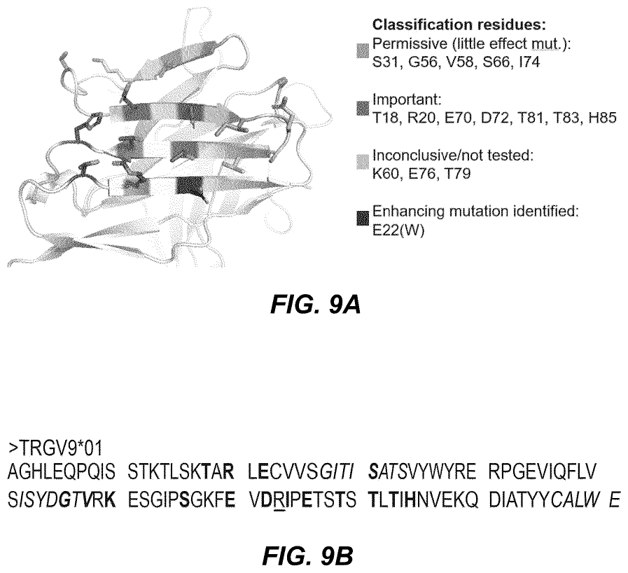

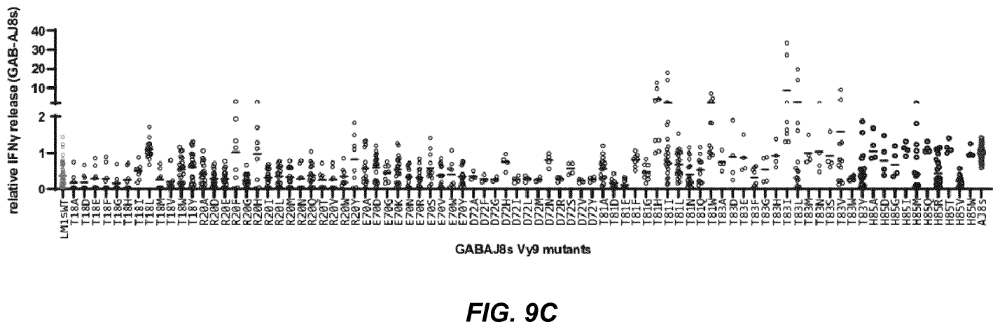

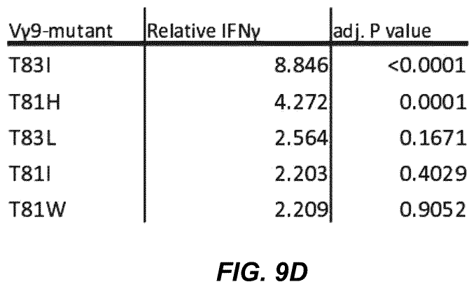

The screening based on the affinity prediction led to only one Vγ9 mutant with significant higher activation potential when using purified GABs. However, the results of screening this panel of Vγ9 mutants provided more insights in the binding interface (FIG. 4B). Several Vγ9 residues could be mutated to a variety of different amino acids without changing the effectivity of the GAB. These residues, S31, G56, V58, S66, and 174, were called permissive residues, and are located at the edge of the BTN2A1 binding interface on the Vγ9 domain (FIG. 9A). Another group of residues, called the important residues, T18, R20, E70, D72, T81, T83, and H85, did not allow for a wide variety of amino acid substations and are thus strongly involved in the interaction with BTN2A1. These residues clustered to together on the Vγ9 domain (FIG. 9A), and also contained the residues previously identified to be important for the interaction with BTN2A1, R20, E70, and H85 [13].

To explore whether additional potency enhancing mutations could be identified in these important residues, a new panel of Vγ9 mutants was generated in GAB-AJ8s, which covered the remaining amino acid substitutions that had not been tested in the initial screen. Again many of the introduced mutations resulted in similar activation as the poorly functional Vγ9Vδ2TCR LM1sGAB (FIG. 9C). However, two mutations had significantly higher IFNγ production compared to GAB-AJ8s, Vγ9 mutations T81H (4.2 mean fold difference) and T83I (8.8 mean fold difference), while several other mutations, Vγ9 mutations T81I, T81W, and T83L, induced a 2-fold increase in IFNγ production (FIG. 9D).

Mutations Vγ9_{T91H} and Vγ9_{T81W} Increase GAB Potency 2-Fold

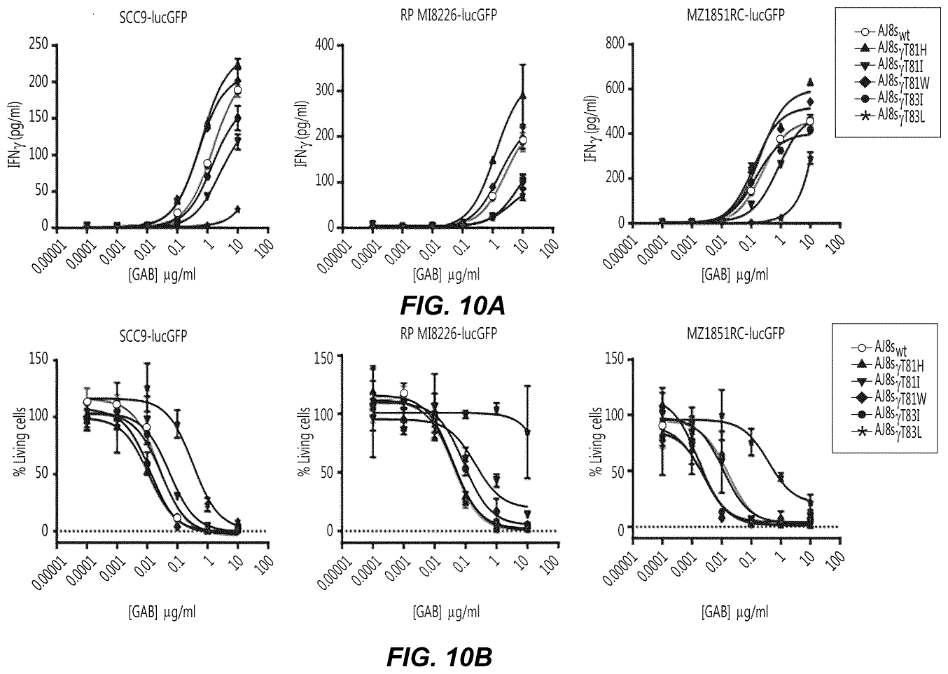

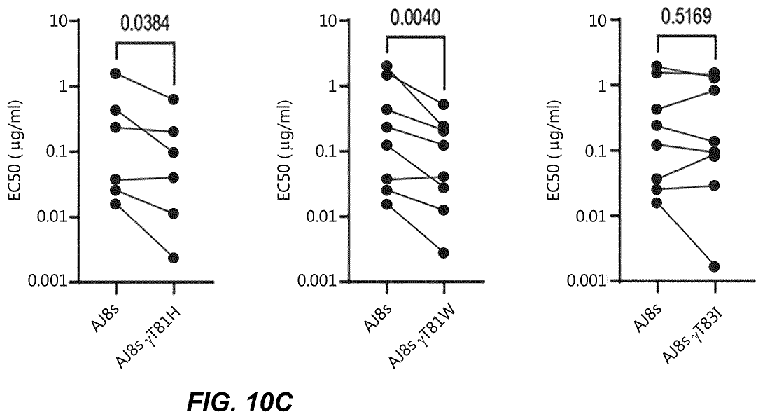

The five selected GAB-AJ8s-Vγ9 mutations, T81H, T81I, T81W, T83I, and T83L, were expressed and in HEK293F cell and purified from the expression media using a 2 step purification strategy. The potency of the GAB-Vγ9 mutants was assessed in co-cultures of T cells and tumor cell lines in the presence of 30 μM pamidronate. GAB-AJ8s_{γT81H} and GAB-AJ8s_{γT81W} show a slight increase in potency compared to GAB-AJ8s when IFNγ secretion by T cells was assessed (FIG. 10A). The other three GAB-Vγ9 mutants, Vγ9 T81I, T83I, and T83L were of comparable or lower potency as GAB-AJ8s (FIG. 10A).

The same trend was observed when T cell mediated tumor cell killing was assessed (FIG. 10B). To formally assess differences in potency, EC50 values were determined for GAB-AJ8s, GAB-AJ8s_{γT81H}, GAB-AJ8s_{γT81W}, and GAB-AJ8s_{γT83I} for each individual experiment. Paired analysis confirmed that GAB-AJ8s_{γT81H} and GAB-AJ8s_{γT81W} have a significant increase in potency compared to GAB-AJ8s, with a mean fold difference of 2.4 and 2.8 in EC50 value, respectively, while GAB-AJ8s_{γT83I} showed no difference in potency (FIG. 10C). This latter observation was unexpected as in the screening with non-purified GAB expression media, GAB-AJ8s_{γT83I} induced the highest fold difference in the relative IFNγ secretion. As mentioned before, expression differences of GAB aggregates in the non-purified expression media could result in false positive signal.

In summary, it was found that the mutation Vγ9_{R73Y} can stabilize protein expression and e.g., enhance yield and expression of GABs, and most likely also stabilize the TCR within the TEG format. It was found that there is an arginine present at this position in over half of the functional Vβ genes, implying that this strategy can be used also for stabilizing Vβ genes for diagnostic and therapeutic purposes. It was confirmed that mutations introduced in Vγ9 residues that have been identified to be crucial for BTN2A1 binding such as R20, E70, and H85 [13], also induced low T cell activation, though interestingly Vγ9-E70 could be replaced by a leucine without any loss in GAB potency. These findings are extended by showing GAB-AJ8s_{γE22W} has, when compared to GAB-AJ8s, a 10-fold increased activity for both IFNγ release and tumor killing. It was also shown that GAB-AJ8s_{γE22W} could induce T cell activation in the absence of pamidronate and that this activation was further enhanced by increasing the pamidronate concentration. Moreover, introducing the Vγ9_{E22W} mutation in the more potent GAB-A3s also increased the activity of this GAB. Next to the bispecific GAB format, αβT cells transduced with Vγ9Vδ2TCRs, show similar behavior, namely the Vγ9_{E22W} mutation in combination with a functional Vγ9Vδ2TCR increased the killing capacity of the these transduced αβT cells. Thus, the Vγ9_{E22W} mutation has the potential to enhance Vγ9Vδ2 TCR-based immune therapies. Finally, two additional mutations were identified that increase the potency of GAB-AJ8 twofold, namely Vγ9_{T91H} and Vγ9_{T81W}.

### Conclusions

Four mutations in the Vγ9-domain were identified that are beneficial for GABs. Vγ9_{R73Y} and increase the GAB production yield twofold, probably by stabilizing the Vγ9 domain. This mutation is most likely also useful for engineering approaches using the β chains. Vγ9_{E22W} increases GAB potency 10 fold and potentiates other Vγ9V2-TCR based therapeutics, like TEGs. Two others mutations, Vγ9_{T91H} and Vγ9_{T81W}, increase the GAB potency twofold. Accordingly, the relationship between substituting specific residues and effect thereof on the capacity of the peptides according to this disclosure to bind BTN2A1 or on its stability is shown in Tables 2 and 3.

TABLE 2
Relationship between substituting specific residues of the BTN2A1
binding peptide according to this disclosure and effect thereof

| | Numbering of | | Effect of conservative |
|---|---|---|---|
| | amino acids | Effect of substitution | substitution of each of |
| | with respect | of each of the amino | the amino acids, i.e., |
| | to SEQ ID | acids by amino acid | substitution by amino |
| | NO: 1/SEQ ID | from other type | acid from same type |
| Region | NO: 43 | group* | group* |
| AGHLEQPQISSTKTLSK . A . L | 1-17, | does not affect binding | does not affect binding |
| (SEQ ID NO: 47) | 19, 21 | to BTN2A1/still | to BTN2A1/still |
| | | binds BTN2A1 | binds BTN2A1 |
| T.R. | 18, 20 | Large negative impact | Medium negative |
| | | binding to BTN2A1/ | impact binding to |
| | | may still bind | BTN2A1/may still |
| | | BTN2A1 | bind BTN2A1 |
| E | 22 | Very large positive | Putative positive |
| | | impact binding to | impact binding to |
| | | BTN2A1 (observed | BTN2A1 |
| | | for E22W and E22F) | |
| CVVSGITISATSVYWYRERP | 23-69 | does not affect binding | does not affect binding |
| GEVIQFLVSISYDGTVRKES | | to BTN2A1/still | to BTN2A1/still |
| GIPSGKF | | binds BTN2A1 | binds BTN2A1 |
| (SEQ ID NO: 48) | | | |
| E (or L). D | 70, 72 | Large negative impact | Medium negative |
| | | binding to BTN2A1/ | impact binding to |
| | | may still bind | BTN2A1/may still |
| | | BTN2A1 | bind BTN2A1 |
| R | 73 | Very large positive | Large positive impact |
| | | impact stability and | stability and does not |
| | | does not affect binding | affect binding to |
| | | to BTN2A1/still | BTN2A1/still binds |
| | | binds BTN2A1 | BTN2A1 |
| T | 81 | Large positive impact | Putative positive |
| | | binding to BTN2A1 | impact binding to |
| | | (observed for T81H | BTN2A1 |
| | | and T81W) | |
| T (or I) . H | 83, 85 | Large negative impact | Medium negative |
| | | binding to BTN2A1/ | impact binding to |
| | | may still bind | BTN2A1/may still |
| | | BTN2A1 | bind BTN2A1 |
| . V .. IPETSTS . L . I. NVE | 71, 74-80, | does not affect binding | does not affect binding |
| KQDIATYYCALWE | 82, 84, | to BTN2A1/still | to BTN2A1/still |
| (SEQ ID NO: 49) | 85-101 | binds BTN2A1 | binds BTN2A1 |

*Type groups of amino acids
Ala, Ser, Thr, Gly (small aliphatic, nonpolar or slightly polar residues)
Asp, Asn, Glu, Gln (polar, negatively charged residues and their amides)
His, Arg, Lys (polar, positively charged residues)
Met, Leu, Ile, Val (Cys) (large aliphatic, nonpolar residues)
Phe, Tyr, Trp (large aromatic residues)

TABLE 3
Relationship between substituting specific residues of the β chain
peptide according to this disclosure and effect thereof

| | Numbering of | | |
|---|---|---|---|
| | amino acids with | | |
| | respect to SEQ ID | | Effect of conservative |
| | NO: 12 (or | Effect of substitution | substitution of each of |
| | corresponding | of each of the amino | the amino acids, i.e., |
| | position in | acids by amino acid | substitution by amino |
| | SEQ ID NO: | from other type | acid from same type |
| Region | 13-42) | group* | group* |
| EPEVTQTPSHQVTQMG | 1-96 | does not affect | does not affect stability |
| QEVILRCVPISNHLYFYW | (excl 69) | stability/still | /still provides |
| YRQILGQKVEFLVSFYNN | | provides adequate | adequate stability |
| EISEKSEIFDDQFSVE . | | stability | |
| PDGSNFTLKIRSTKLED | | | |
| SAMYFCASSE | | | |
| (SEQ ID NO: 50) | | | |
| R | 69 | Very large positive | Putative positive |
| | | impact stability, | impact stability |
| | | especially for large | |
| | | aromatic residues | |

*Type groups of amino acids
Ala, Ser, Thr, Gly (small aliphatic, nonpolar or slightly polar residues)
Asp, Asn, Glu, Gln (polar, negatively charged residues and their amides)
His, Arg, Lys (polar, positively charged residues)
Met, Leu, Ile, Val (Cys) (large aliphatic, nonpolar residues)
Phe, Tyr, Trp (large aromatic residues)

### Example 2

### Substitution of Vγ9-E22 by Phenylalanine Increases Potency of GAB-AJ8s

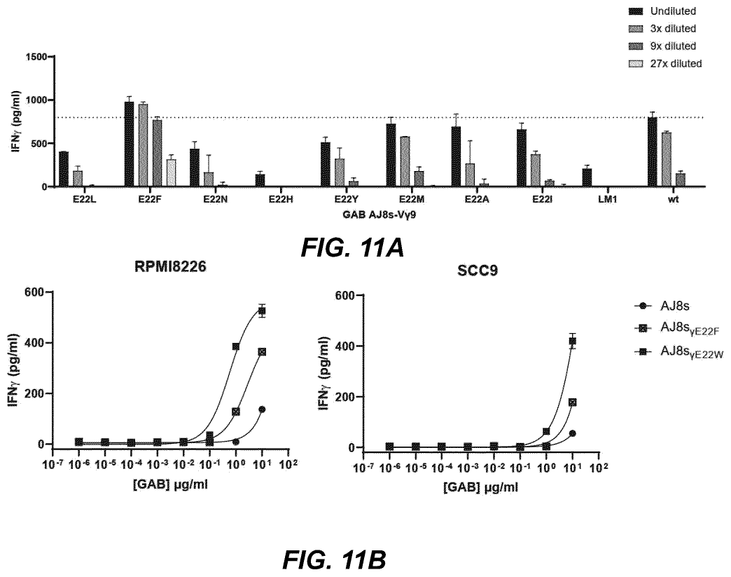

Above, Vγ9_{E22W} was identified as one of the mutations that increase the potency of GABs and TEGs, however the number of substitutions tested at this position was limited. This number was extended by introducing eight more substitutions at position Vγ9_{E22} using the golden gate cloning strategy described above. These GAB-AJ8s variants were expressed in HEK293F cells and the expression media was used in a T cell activation assay. After 20 hours of coculture of T cells, MZ1851RC tumors cells, 30 μM pamidronate and GAB expression media, the levels of IFNγ in the coculture media were determined using an ELISA. Out of these eight GAB-AJ8s-Vγ9E22-mutants, particularly one mutant, GAB-AJ8s-Vγ9_{E22F}, outperformed GAB-AJ8s (FIG. 11A). Several other hydrophobic substitutions, like alanine, leucine, isoleucine, methionine, and tyrosine, led to similar or slightly impaired activity compared to GAB-AJ8s, while a histidine on position 22 completely abrogated activity (FIG. 11A). However, using unpurified GAB expression media can lead to slightly skewed results, as no corrections were made for differences in expressions levels or aggregates. Therefore, GAB-AJ8s-Vγ9_{E22F} was expressed and purified and assessed for its potency alongside purified GAB-AJ8s and GAB-AJ8s_{γE22W}, using an IFNγ release assay using RPMI8226 and SCC9 as tumor targets. The substitution of glutamic acid at position 22 by phenylalanine results in a more potent GAB-AJ8s, but does not outperform GAB-AJ8s_{γE22W} (FIG. 11B).

### Certain Vδ2-Mutants Enhance the Potent of GAB-AJ8s

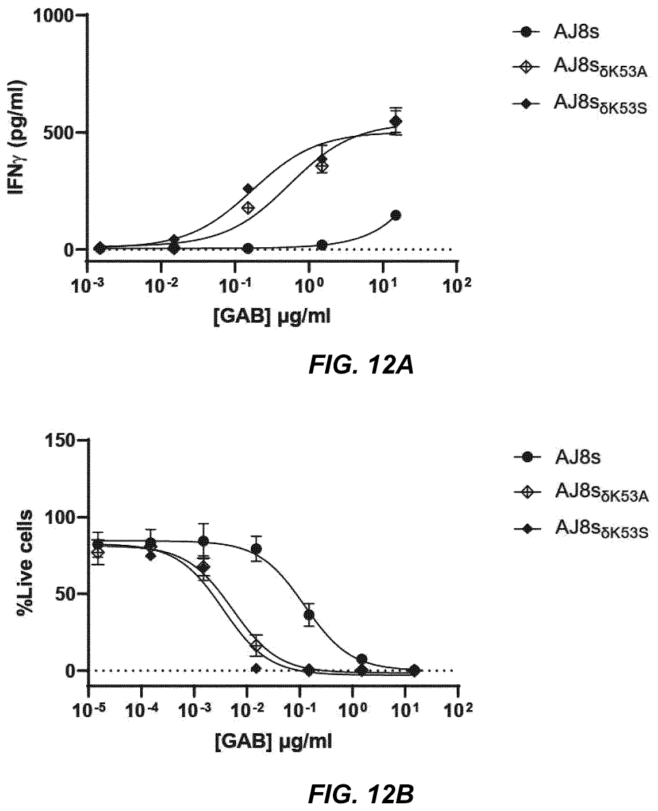

Recently, affinity enhancing mutations in Vδ2-CDR2, residue K53, were disclosed [72 and patent document WO2023102615A1]. In order to determine whether mutations at this position would also enhance the potency of GAB-AJ8s, mutations K53S and K53A, were introduced in the AJ86 chain, and GAB-AJ8s_{δK53S} and GAB-AJ8s_{δK53A} were expressed in HEK293F and purified as described above. The purified GABs were used in activity assays using RPMI8226 as tumor target. In line with the reported increased affinity of these Vδ2-K53 mutants, both GAB-AJ8s_{δK53S} and GAB-AJ8s_{δK53A} were more potent compared to GAB-AJ8s in inducing T cell activation, measured by IFNγ release (FIG. 12A), and T cell mediated tumor cell killing (FIG. 12B). No substantial differences in potency were observed between the two Vδ2-K53 mutants in these assays.

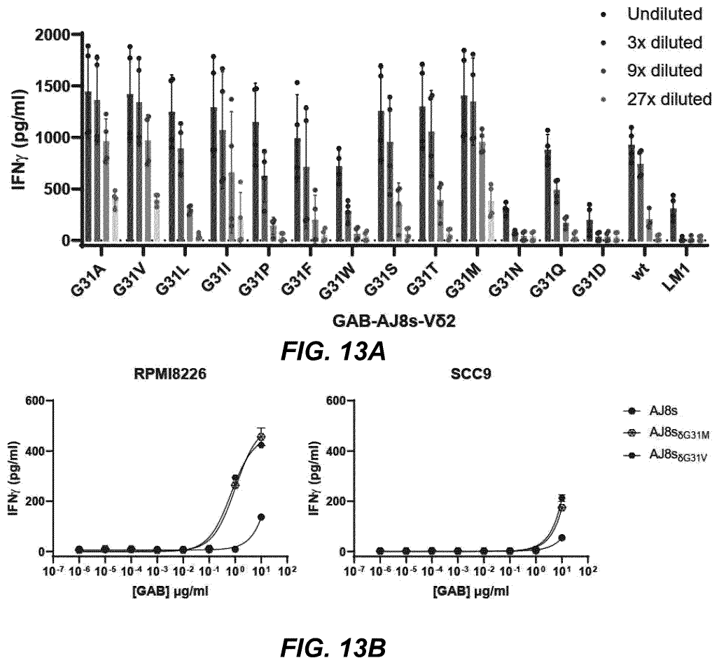

The impact of residues in the Vδ2-CDR1 have also been addressed in the past by limited mutagenesis or by combining human and primate Vγ9 and Vδ2 chains [73, 74]. The Vδ2-CDR1 is fairly conserved among primates, except for Vδ2-residue 31, which is a glycine in human (Homo sapiens), chimpanzee (Pan troglodytes), and in some gorilla (Gorilla gorilla gorilla), but a serine or threonine in other primate species (see “Alignment TRDV2” as disclosed herein). To gain a better understanding of which amino acids at Vδ2-residue 31 could lead to functional GAB-AJ8s, mutations at Vδ2-residue 31 were introduced in a GAB expression vector containing GAB-AJ8s-Vδ2-CDR1_2acc using golden-gate cloning, as described for the Vγ9-mutants above. After sequence verification, the expression plasmids were used to transfect HEK293F cells for GAB production. After five days of expression the supernatant was collected and used in coculture assays with donor derived T cells and MZ1851RC cancer cell line to assess the dose dependent activation of T cells by GABs. Substitution to small polar amino acids, GAB-AJ8s_{δG31S} and GAB-AJ8s_{δG31T}, that are used in primate Vδ2 chains, led to similar activity compared to GAB-AJ8s, whereas the use of bulkier polar amino acids, like asparagine or glutamine, or charged amino acids, like aspartic acid, led to decreased activity (FIG. 13A). Substituting Vδ2-G31 by any hydrophobic amino acids (alanine, valine, leucine, isoleucine, or methionine), led to similar or increased potency of the GAB-AJ8s-variant compared to GAB-AJ8s, an indication that at this position a hydrophobic amino acid was preferred (FIG. 13A). To confirm that this increase in potency was caused by the introduced mutation and not by expression differences, a small or large hydrophobic amino acid, valine or methionine, respectively, was used as substitution for glycine. These GAB-AJ8s-variants, GAB-AJ8s_{δG31V} and GAB-AJ8s_{δG31M}, were expressed, purified, and used in a T cell activation assay alongside GAB-AJ8s to assess their potency under controlled conditions, using RPMI8226 and SCC9 as tumor targets. Both AJ8s_{δG31V} and GAB-AJ8s_{δG31M} were more potent compared to GAB-AJ8s as shown by increased IFNγ release by T cells at lower GAB concentration after coculture with tumor cells (FIG. 13B). In conclusion, mutating Vδ2-G31 to a hydrophobic amino acid results in GAB, or any other Vδ2-based therapeutic, with increased potency.

Combinations of Vγ9E22W with Potency Enhancing Vδ2-Mutants Results in GAB with Superior Efficacy

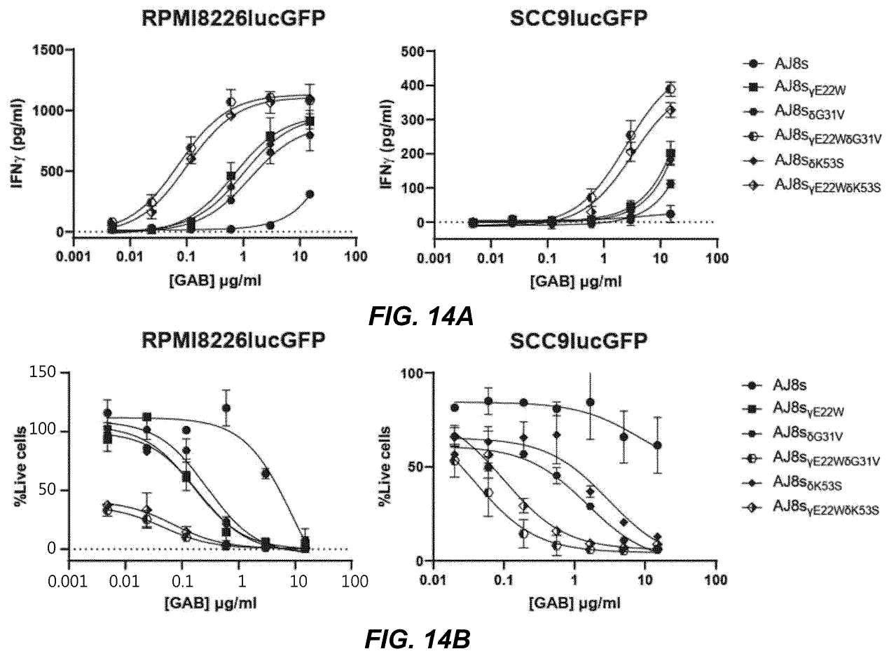

Vγ9_{E22W} does not only increase the potency of GAB-AJ8s but also of GAB-A3s, that is based on the intrinsically more potent TCR A3 [75, 76], as shown above (FIG. 7). However, TCR-A3 consist of germline Vδ2-CDR1 and -CDR2 sequences, so it remains a question if affinity enhancing mutations in these regions combined with Vγ9E22W would lead to GABs with even higher potency. To address this, GABs containing affinity enhancing mutations in both the Vγ9 and Vδ2 chain were expressed and purified, and tested alongside the single mutations for their ability to activate T cells when cocultured with tumor cells. The panel of GABs to be tested consisted of: GAB-AJ8s, GAB-AJ8s_{γE22W}, AJ8s_{δG31V}, GAB-AJ8s_{γE22WδG31V}, GAB-AJ8s_{δK53S}, and GAB-AJ8s_{γE22WδK53S}. First, the IFNγ level after 20 hours of coculture was measured using an ELISA. All single mutations, GAB-AJ8s_{γE22W}, AJ8s_{δG31V}, and GAB-AJ8s_{δK53S}, were of comparable potency and, as established above, they were an order of magnitude more potent with respect to GAB-AJ8s (FIG. 14A). The combined Vγ9 and Vδ2 mutations in GAB-AJ8s_{γE22WδG31V} and GAB-AJ8s_{γE22WδK53S} resulted in GAB variants that were 10 times more potent than the single mutants. These differences were seen for both hematological and solid tumor cell lines, RPMI8226 and SCC9, respectively. Lastly, T cell induced cytotoxicity was determined using luciferase expressing tumor cells. In line with the results obtained using the IFNγ release assay, GAB-AJ8s_{γE22WδG31V} and GAB-AJ8s_{γE22WδK53S} were, again, over 10-fold more potent than the single Vγ9 or Vδ2 mutants and over 100-fold more potent compared to GAB-AJ8s in T cell mediated killing of RPMI8226 and SCC9 tumor cells (FIG. 14B).

### Conclusions

Affinity maturation has been used for decades to improve the efficacy and potency of therapeutic proteins, like antibodies and αβTCRs. Here, the affinity of the Vγ9 chain to BTN2A1 and the Vδ2 chain to BTN3 Å has been enhanced by mutagenesis. Combinations of the most potent Vγ9 chain mutant E22W with Vδ2 chain mutant G31V or K53S in the GAB format, led to GABs with unprecedented potency. Most likely, triple or quadruple mutants, combining Vγ9 mutations E22W and/or T81H/W with the here described Vδ2 mutations at positions G31 and K53 would lead to even be more potent GABs or TEGs.

### REFERENCES

1. Waldman, A. D., J. M. Fritz, and M. J. Lenardo, A guide to cancer immunotherapy: from T cell basic science to clinical practice. Nat Rev Immunol, 2020. 20(11): p. 651-668.
2. Goebeler, M. E. and R. C. Bargou, T cell-engaging therapies—BiTEs and beyond. Nat Rev Clin Oncol, 2020. 17(7): p. 418-434.
3. McMurray, J. L., et al., Transcriptional profiling of human Vdelta1 T cells reveals a pathogen-driven adaptive differentiation program. Cell Rep, 2022. 39(8): p. 110858.
4. Willcox, B. E. and C. R. Willcox, gammadelta TCR ligands: the quest to solve a 500-million-year-old mystery. Nat Immunol, 2019. 20(2): p. 121-128.
5. Uldrich, A. P., M. Rigau, and D. I. Godfrey, Immune recognition of phosphoantigen-butyrophilin molecular complexes by gammadelta T cells. Immunol Rev, 2020. 298(1): p. 74-83.
6. Exley, M. A., P. Dellabona, and G. Casorati, Exploiting CD1-restricted T cells for clinical benefit. Mol Immunol, 2021. 132: p. 126-131.
7. Keller, A N., et al., MAIT cells and MR1-antigen recognition. Curr Opin Immunol, 2017. 46: p. 66-74.
8. Sebestyen, Z., et al., Translating gammadelta (gammadelta) T cells and their receptors into cancer cell therapies. Nat Rev Drug Discov, 2020. 19(3): p. 169-184.
9. Morita, C. T., et al., Nonpeptide antigens, presentation mechanisms, and immunological memory of human Vgamma2Vdelta2 T cells: discriminating friend from foe through the recognition of prenyl pyrophosphate antigens. Immunol Rev, 2007. 215: p. 59-76.
10. Harly, C., et al., Key implication of CD277 butyrophilin-3 (BTN3A) in cellular stress sensing by a major human gammadelta T-cell subset. Blood, 2012. 120(11): p. 2269-2279.
11. Sandstrom, A., et al., The intracellular B30.2 domain of butyrophilin 3A1 binds phosphoantigens to mediate activation of human Vgamma9Vdelta2 T cells. Immunity, 2014. 40(4): p. 490-500.
12. Hsiao, C. C., et al., Ligand-induced interactions between butyrophilin 2A1 and 3A1 internal domains in the HMBPP receptor complex. Cell Chem Biol, 2022. 29(6): p. 985-995 e5.
13. Rigau, M., et al., Butyrophilin 2A1 is essentialfor phosphoantigen reactivity by gammadelta T cells. Science, 2020. 367(6478).
14. Karunakaran, M. M., et al., Butyrophilin-2A1 Directly Binds Germline-Encoded Regions of the Vgamma9Vdelta2 TCR and Is Essential for Phosphoantigen Sensing. Immunity, 2020. 52(3): p. 487-498 e6.
15. Rossjohn, J., et al., T cell antigen receptor recognition of antigen-presenting molecules. Annu Rev Immunol, 2015. 33: p. 169-200.
16. Vyborova, A., et al., gamma9delta2T cell diversity and the receptor interface with tumor cells. J Clin Invest, 2020. 130(9): p. 4637-4651.
17. Lai, A. Y., et al., Cutting Edge: Bispecific gammadelta T Cell Engager Containing Heterodimeric BTN2A1 and BTN3A1 Promotes Targeted Activation of Vgamma9Vdelta2(+) T Cells in the Presence of Costimulation by CD28 or NKG2D. J Immunol, 2022. 209(8): p. 1475-1480.
18. Wang, H., Z. Fang, and C. T. Morita, Vgamma2Vdelta2 T Cell Receptor recognition of prenyl pyrophosphates is dependent on all CDRs. J. Immunol., 2010. 184(11): p. 6209-6222.
19. Grunder, C., et al., gamma9 and delta2CDR3 domains regulate functional avidity of T cells harboring gamma9delta2TCRs. Blood, 2012. 120(26): p. 5153-5162.
20. van Diest, E., et al., Gamma delta TCR anti-CD3 bispecific molecules (GABs) as novel immunotherapeutic compounds. J Immunother Cancer, 2021. 9(11).
21. Marcu-Malina, V., et al., Redirecting alphabeta T cells against cancer cells by transfer of a broadly tumor-reactive gammadeltaT-cell receptor. Blood, 2011. 118(1): p. 50-59.
22. Straetemans, T., et al., Untouched GMP-Ready Purified Engineered Immune Cells to Treat Cancer. Clin Cancer Res, 2015. 21(17): p. 3957-68.
23. Zheng, J., et al., A novel antibody-like TCRgammadelta-Ig fusion protein exhibits antitumor activity against human ovarian carcinoma. Cancer Lett, 2013. 341(2): p. 150-8.
24. Stone, J. D., D. T. Harris, and D. M. Kranz, TCR affinity for p/MHC formed by tumor antigens that are self-proteins: impact on efficacy and toxicity. Curr Opin Immunol, 2015. 33: p. 16-22.
25. D'Ippolito, E., et al., T cell engineering for adoptive T cell therapy: safety and receptor avidity. Cancer Immunol Immunother, 2019. 68(10): p. 1701-1712.
26. Spear, T. T., et al., Understanding TCR affinity, antigen specificity, and cross-reactivity to improve TCR gene-modified T cells for cancer immunotherapy. Cancer Immunol Immunother, 2019. 68(11): p. 1881-1889.
27. Kramer, L., et al., Challenges of γ9δ2TCR affinity maturation when using phage display. bioRxiv, 2021: p. 2021.05.20.445024.
28. van Zundert, G. C. P., et al., The HADDOCK2.2 Web Server: User-Friendly Integrative Modeling of Biomolecular Complexes. J Mol Biol, 2016. 428(4): p. 720-725.
29. Wassenaar, T. A., et al., WeNMR: Structural Biology on the Grid. Journal of Grid Computing, 2012. 10(4): p. 743-767.
30. Kelley, L. A., et al., The Phyre2 web portal for protein modeling, prediction and analysis. Nat Protoc, 2015. 10(6): p. 845-58.
31. Huang, X., et al., SSIPe: accurately estimating protein-protein binding affinity change upon mutations using evolutionary profiles in combination with an optimized physical energy function. Bioinformatics, 2020. 36(8): p. 2429-2437.
32. Pearce, R., et al., EvoDesign: Designing Protein-Protein Binding Interactions Using Evolutionary Interface Profiles in Conjunction with an Optimized Physical Energy Function. J Mol Biol, 2019. 431(13): p. 2467-2476.
33. Berliner, N., et al., Combining structural modeling with ensemble machine learning to accurately predict protein fold stability and binding affinity effects upon mutation. PLoS One, 2014. 9(9): p. e107353.
34. Strokach, A., T. Y. Lu, and P. M. Kim, ELASPIC2 (EL2): Combining Contextualized Language Models and Graph Neural Networks to Predict Effects of Mutations. J Mol Biol, 2021. 433(11): p. 166810.
35. Rodrigues, C. H. M., et al., mCSM-PPI2: predicting the effects of mutations on protein-protein interactions. Nucleic Acids Res, 2019. 47(W1): p. W338-W344.
36. Geng, C. L., et al., Finding the Delta Delta G spot: Are predictors of binding affinity changes upon mutations in protein-protein interactions ready for it? Wiley Interdisciplinary Reviews-Computational Molecular Science, 2019. 9(5).
37. Zhou, H. X. and X. Pang, Electrostatic Interactions in Protein Structure, Folding, Binding, and Condensation. Chem Rev, 2018. 118(4): p. 1691-1741.
38. Isom, D. G., et al., High tolerance for ionizable residues in the hydrophobic interior of proteins. Proc Natl Acad Sci USA, 2008. 105(46): p. 17784-8.
39. Harms, M. J., et al., Arginine residues at internal positions in a protein are always charged. Proc Natl Acad Sci USA, 2011. 108(47): p. 18954-9.
40. Lefranc, M. P., et al., IMGT®, the international ImMunoGeneTics information System® 25 years on. Nucleic Acids Res, 2015. 43(Database issue): p. D413-22.
41. Engler, C., R. Kandzia, and S. Marillonnet, A one pot, one step, precision cloning method with high throughput capability. PLoS One, 2008. 3(11): p. e3647.
42. Shi et al., Mol Cancer. 2014 Sep. 21; 13:219
43. Sebestyen et al., 2016. Cell Rep 15: 1973-85
44. Gu et al., 2017. PNAS 114: E7311-E20
45. Sandstrom et al., 2014. Immunity 40: 490-500
46. Dudley et al., 2008. J Clin Oncol 26: 5233-9
47. Claudio Tripodo et al. Gamm delta T cell lymphomas Nature Reviews Clinical Oncology 6, 707-717 (December 2009)
48. COMPUTATIONAL MOLECULAR BIOLOGY, Lesk, A. M., ed., Oxford University Press, New York, 1988
49. BIOCOMPUTING: INFORMATICS AND GENOME PROJECTS, Smith, D. W., ed., Academic Press, New York, 1993
50. COMPUTER ANALYSIS OF SEQUENCE DATA, PART I, Griffin, A. M., and Griffin, H. G., eds., Humana Press, New Jersey, 1994
51. SEQUENCE ANALYSIS IN MOLECULAR BIOLOGY, von Heinje, G., Academic Press, 1987
52. SEQUENCE ANALYSIS PRIMER; Gribskov, M. and Devereux, J., eds., M Stockton Press, New York, 1991
53. Carillo, H., and Lipton, D., SIAM J. Applied Math (1988) 48:1073
54. GUIDE TO HUGE COMPUTERS, Martin J. Bishop, ed., Academic Press, San Diego, 1994
55. Carillo, H., and Lipton, D., SIAM J. Applied Math (1988) 48:1073
56. Devereux, J., et al., Nucleic Acids Research (1984) 12(1):387
57. Atschul, S. F. et al., J. Molec. Biol. (1990) 215:403
58. Koerber et al, 2015. J Mol Biol 427: 576-86
59. Skerra, 2007. Current Opinion Biotechnol 18: 295-304
60. Skrlec et al, 2015. Trends Biotechnol 33: 408-418
61. Liao et al., 2000. Gene Ther 7: 339-47
62. Arakawa et al., 1996. J Biochem 120: 657-62
63. Adair et al., 1994. Human Antibodies 5: 41-47
64. Kipriyanov et al., 1997. Protein Engin Design Selection 10: 445-453
65. Levine et al., 2017. Mol Ther Methods Clin Dev 4: 92-101
66. Fallaux, F. J. et al. 1998. Hum Gene Ther 9: 1909-1917
67. Schulz, G. E. et al, Principles of Protein Structure, Springer-Verlag, New York, 1979, and Creighton, T. E., Proteins: Structure and Molecular Principles, W. H. Freeman & Co., San Francisco, 1984
68. Kyte et al., J. Mol. Biol. 157, 105-132 (1982)
69. Sambrook et al. (1989) Molecular Cloning: A Laboratory Manual, Second Edition, Cold Spring Harbor Laboratory Press
70. Sambrook and Russell (2001) Molecular Cloning: A Laboratory Manual, Third Edition, Cold Spring Harbor Laboratory Press, NY
71. Volumes 1 and 2 of Ausubel et al. (1994) Current Protocols in Molecular Biology, Current Protocols, USA
72. Fulford, T. S., et al., Vγ9Vδ2 T cells recognize butyrophilin 2A1 and 3A1 heteromers. bioRxiv, 2023: p. 2023.08.30.555639.
73. Wang, H., Z. Fang, and C. T. Morita, Vgamma2Vdelta2 T Cell Receptor recognition of prenyl pyrophosphates is dependent on all CDRs. J Immunol, 2010. 184(11): p. 6209-22.
74. Wang, H., et al., Conservation of nonpeptide antigen recognition by rhesus monkey Vgamma 2V delta 2 T cells. J Immunol, 2003. 170(7): p. 3696-706.
75. Vyborova, A., et al., γ9δ2T cell diversity and the receptor interface with tumor cells. J Clin Invest, 2020.
76. van Diest, E., et al., Gamma delta TCR anti-CD3 bispecific molecules (GABs) as novel immunotherapeutic compounds. J Immunother Cancer, 2021. 9(11).
77. Nature Communications volume 14, Article number: 7617 (2023)

1. A Butyrophilin Subfamily 2 Member A1 (BTN2A1) binding peptide comprising an amino acid sequence that binds BTN2A1, wherein the amino acid sequence has at least 70% sequence identity with SEQ ID NO: 1, wherein the amino acid sequence comprises:
a tryptophan or phenylalanine at a position corresponding to position 22 as shown in SEQ ID NO: 1;
a tyrosine or conservative substitution thereof chosen from tryptophan and phenylalanine at a position corresponding to position 73 as shown in SEQ ID NO: 1; and/or
a histidine or conservative substitution thereof chosen from arginine and lysine, or tryptophan or conservative substitution thereof chosen from phenylalanine and tyrosine at a position corresponding to position 81 as shown in SEQ ID NO: 1.

2. The BTN2A1 binding peptide of claim 1, which comprises:
a threonine or conservative substitution thereof chosen from alanine, serine, glycine and leucine at a position corresponding to position 18 as shown in SEQ ID NO: 1;
an arginine or conservative substitution thereof chosen from histidine, and lysine at a position corresponding to position 20 as shown in SEQ ID NO: 1;
a glutamic acid or conservative substitution thereof chosen from aspartic acid, asparagine, and glutamine, or leucine or conservative substitution thereof chosen from methionine, isoleucine, valine and cysteine at a position corresponding to position 70 as shown in SEQ ID NO: 1;
an aspartic acid or asparagine, at a position corresponding to position 72 as shown in SEQ ID NO: 1;
a threonine or conservative substitution thereof chosen from alanine, serine and glycine, or a histidine or conservative substitution thereof chosen from arginine and lysine, or tryptophan or conservative substitution thereof chosen from phenylalanine and tyrosine at a position corresponding to position 81 as shown in SEQ ID NO: 1;
a threonine or conservative substitution thereof chosen from alanine, serine and glycine or isoleucine or conservative substitution thereof chosen from methionine, leucine, valine and cysteine at a position corresponding to position 83 as shown in SEQ ID NO: 1; and/or
a histidine at a position corresponding to position 85 as shown in SEQ ID NO: 1.

3. The BTN2A1 binding peptide of claim 1, wherein the amino acid sequence has at least 80% sequence identity with SEQ ID NO:1.

4. The BTN2A1 binding peptide of claim 1, which is a T-cell receptor γ-chain.

5. Tie BTN2A1 binding peptide of claim 1, which is in combination with a Butyrophilin Subfamily 3 Member A1 (BTN3A1) and/or BTN3A2 and/or BTN3A3 binding peptide comprising an amino acid sequence that binds BTN3A1 and/or BTN3A2 and/or BTN3A3, wherein the amino acid sequence has at least 70% sequence identity with SEQ ID NO:52, wherein the amino acid sequence comprises:
a valine, methionine, alanine, isoleucine or leucine at a position corresponding to position 31 as shown in SEQ ID NO:52; and/or
a serine or alanine at a position corresponding to position 53 as shown in SEQ ID NO:52.

6. The BTN2A1 binding peptide of claim 1, which is comprised in an (exogeneous) immune receptor or extracellular domain thereof.

7. A construct comprising:
i) γδ T-cell receptor or extracellular domain thereof according to claim 6; and
ii) a toxin and/or a label.

8. A construct comprising:
i) γδ T-cell receptor or extracellular domain thereof according to claim 6; and
ii) an effector cell binding domain.

9. The construct according to claim 8, wherein
a T-cell binding domain binds CD3, CD4, CD8, CD 16, CD56, CD103, CD134, CD154 and/or CD314; and/or is a single chain Fv anti-CD3, CD4, CD8, CD 16, CD56, CD103, CD134, CD154 and/or CD314 binding domain; and/or
a Natural Killer (NK) cell-binding domain binds CD16, NKG2D, NKp30, NKp44, NKp46, and/or DNAM; and/or is a single chain Fv anti-CD16, NKG2D, NKp30, NKp44, NKp46, and/or DNAM binding domain.

10. The construct according to claim 8, wherein
a T-cell binding domain binds PD1, LAG3, CTLA4, TIGIT, CD96, BTLA, VISTA, TIM3, LAIR1, (inhibitory) KIR, CD160 and/or immune receptor with an intracellular ITIM or ITSM motif; and/or is a single chain Fv anti-PD1, LAG3, CTLA4, TIGIT, CD96, BTLA, VISTA, TIM3, LAIR1, (inhibitory) KIR, CD160 and/or immune receptor with an intracellular ITIM or ITSM motif binding domain; and/or
a Natural Killer (NK) cell binding domain binds NKG2A, CD96, TIGIT, (inhibitory) KIR, PD1, TIM3, LAG3, CD112R, CD160, LAIR1 and/or immune receptor with an intracellular ITIM or ITSM motif and/or is a single chain Fv anti-NKG2A, CD96, TIGIT, (inhibitory) KIR, PD1, TIM3, LAG3, CD 112R, CD160, LAIR1 and/or immune receptor with an intracellular ITIM or ITSM motif binding domain.

11. The construct of claim 7, wherein the construct is a fusion protein.

12. A nucleic acid or nucleic acid combination encoding the BTN2A1 binding peptide of claim 1.

13. A cell that expresses the nucleic acid or nucleic acid combination according to claim 12.

14. The cell of claim 13, wherein the cell is an immune cell.

15. A method of producing the BTN2A1 binding peptide of claim 1, the method comprising expressing a nucleic acid or nucleic acid combination that encodes the BTN2A1 binding peptide in a host cell thereby producing the BTN2A1 binding peptide.

16. A pharmaceutical composition comprising the BTN2A1 binding peptide of claim 1.

17. (canceled)

18. (canceled)

19. A nucleic acid or nucleic acid combination encoding the construct of claim 7.

20. A method of producing the construct of claim 7, the method comprising: expressing a nucleic acid or nucleic acid combination that encodes the construct in a host cell thereby producing the construct.

21. A pharmaceutical composition comprising:
the construct of claim 7.

22. A pharmaceutical composition comprising:
the cell of claim 13.


SEQUENCE LISTING (69 sequences)

SEQ ID NO: 1 - protein, 101 aa, unidentified
AGHLEQPQISSTKTLSKTARLECVVSXXXXXXXXVYWYRERPGEVIQFLVSXXXXXXXRKESGIPSGKFEVDRIPETSTSTLTIHNVEKQDIATYYXXXXX

SEQ ID NO: 2 - protein, 101 aa, unidentified
AGHLEQPQISSTKTLSKTARLECVVSGITISATSVYWYRERPGEVIQFLVSISYDGTVRKESGIPSGKFEVDYIPETSTSTLTIHNVEKQDIATYYCALWE

SEQ ID NO: 3 - protein, 101 aa, unidentified
AGHLEQPQISSTKTLSKTARLWCVVSGITISATSVYWYRERPGEVIQFLVSISYDGTVRKESGIPSGKFEVDYIPETSTSTLTIHNVEKQDIATYYCALWE

SEQ ID NO: 4 - protein, 101 aa, unidentified; identical to SEQ ID NO: 3

SEQ ID NO: 5 - protein, 101 aa, unidentified
AGHLEQPQISSTKTLSKTARLECVVSGITISATSVYWYRERPGEVIQFLVSISYDGTVRKESGIPSGKFEVDYIPETSTSWLTIHNVEKQDIATYYCALWE

SEQ ID NO: 6 - protein, 101 aa, unidentified
AGHLEQPQISSTKTLSKTARLWCVVSGITISATSVYWYRERPGEVIQFLVSISYDGTVRKESGIPSGKFEVDRIPETSTSTLTIHNVEKQDIATYYCALWE

SEQ ID NO: 7 - DNA, 408 bp, unidentified
  source 1..408: mol_type = unassigned DNA
agatctccgccgccaccatggcatgccctggcttcctgtgggcacttgtgatctccacctgtcttgaattttccatggcccgtacgggcgccggacacctggaacagccccagatcagcagcaccaagaccctgagcaagaccgccaggctggaatgcgtggtgtccggcatctgagacgtttcctgatggactgacgtctcatccgtgtactggtacagagagagacccggcgaggtcatccagttcctggtgtccatcagctacgacggcaccgtgcggaaagagagcggcatccccagcggcaagttcgaggtggactacatccccgagaccagcacctccaccctgaccatccacaacgtggagaagcaggacatcgccacctactactgcgccctgtgggagg

SEQ ID NO: 8 - DNA, 408 bp, unidentified
  source 1..408: mol_type = unassigned DNA
agatctccgccgccaccatggcatgccctggcttcctgtgggcacttgtgatctccacctgtcttgaattttccatggcccgtacgggcgccggacacctggaacagccccagatcagcagcaccaagaccctgagcaagaccgccaggctggaatgcgtggtgtccggcatcaccatcagcgccacctccgtgtactggtacagagagagacccggcgaggtcatccagttcctggtgtccatcagctacgactgagacgtttcctgatggactgacgtctcagagagcggcatccccagcggcaagttcgaggtggactacatccccgagaccagcacctccaccctgaccatccacaacgtggagaagcaggacatcgccacctactactgcgccctgtgggagg

SEQ ID NO: 9 - DNA, 408 bp, unidentified
  source 1..408: mol_type = unassigned DNA
agatctccgccgccaccatggcatgccctggcttcctgtgggcacttgtgatctccacctgtcttgaattttccatggcccgtacgggcgccggacacctggaacagccccagatcagcagcaccaagaccctgagcaagaccgccaggctggaatgcgtggtgtccggcatcaccatcagcgccacctccgtgtactggtacagagagagacccggcgaggtcatccagttcctggtgtccatcagctacgacggcaccgtgcggaaagagagcggcatccccagagacgtttcctgatggactgacgtctcagtggactacatccccgagaccagcacctccaccctgaccatccacaacgtggagaagcaggacatcgccacctactactgcgccctgtgggagg

SEQ ID NO: 10 - DNA, 408 bp, unidentified
  source 1..408: mol_type = unassigned DNA
agatctccgccgccaccatggcatgccctggcttcctgtgggcacttgtgatctccacctgtcttgaattttccatggcccgtacgggcgccggacacctggaacagccccagatcagcagcaccaagaccctgagcaagaccgccaggctggaatgcgtggtgtccggcatcaccatcagcgccacctccgtgtactggtacagagagagacccggcgaggtcatccagttcctggtgtccatcagctacgacggcaccgtgcggaaagagagcggcatccccagcggcaagttcgaggtgtgagacgtttcctgatggactgacgtctcaaccagcacctccaccctgaccatccacaacgtggagaagcaggacatcgccacctactactgcgccctgtgggagg

SEQ ID NO: 11 - DNA, 408 bp, unidentified
  source 1..408: mol_type = unassigned DNA
agatctccgccgccaccatggcatgccctggcttcctgtgggcacttgtgatctccacctgtcttgaattttccatggcccgtacgggcgccggacacctggaacagccccagatcagcagcaccaagaccctgagcaagaccgccaggctggaatgcgtggtgtccggcatcaccatcagcgccacctccgtgtactggtacagagagagacccggcgaggtcatccagttcctggtgtccatcagctacgacggcaccgtgcggaaagagagcggcatccccagcggcaagttcgaggtggactacatccccgagaccagcacctccagagacgtttcctgatggactgacgtctcaaacgtggagaagcaggacatcgccacctactactgcgccctgtgggagg

SEQ ID NO: 12 - protein, 96 aa, unidentified
EPEVTQTPSHQVTQMGQEVILRCVPISNHLYFYWYRQILGQKVEFLVSFYNNEISEKSEIFDDQFSVERPDGSNFTLKIRSTKLEDSAMYFCASSE

SEQ ID NO: 13 - protein, 95 aa, unidentified
NAGVTQTPKFQVLKTGQSMTLQCAQDMNHNSMYWYRQDPGMGLRLIYYSASEGTTDKGEVPNGYNVSRLNKREFSLRLESAAPSQTSVYFCASSE

SEQ ID NO: 14 - protein, 95 aa, unidentified
NAGVTQTPKFRVLKTGQSMTLLCAQDMNHEYMYWYRQDPGMGLRLIHYSVGEGTTAKGEVPDGYNVSRLKKQNFLLGLESAAPSQTSVYFCASSY

SEQ ID NO: 15 - protein, 95 aa, unidentified; identical to SEQ ID NO: 14

SEQ ID NO: 16 - protein, 95 aa, unidentified
IAGITQAPTSQILAAGRRMTLRCTQDMRHNAMYWYRQDLGLGLRLIHYSNTAGTTGKGEVPDGYSVSRANTDDFPLTLASAVPSQTSVYFCASSD

SEQ ID NO: 17 - protein, 95 aa, unidentified
NAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSY

SEQ ID NO: 18 - protein, 95 aa, unidentified
NAGVTQTPKFRILKIGQSMTLQCTQDMNHNYMYWYRQDPGMGLKLIYYSVGAGITDKGEVPNGYNVSRSTTEDFPLRLELAAPSQTSVYFCASSY

SEQ ID NO: 19 - protein, 94 aa, unidentified
NAGVTQTPKFHILKTGQSMTLQCAQDMNHGYMSWYRQDPGMGLRLIYYSAAAGTTDKEVPNGYNVSRLNTEDFPLRLVSAAPSQTSVYLCASSY

SEQ ID NO: 20 - protein, 95 aa, unidentified
NAGVTQTPKFHILKTGQSMTLQCAQDMNHGYLSWYRQDPGMGLRRIHYSVAAGITDKGEVPDGYNVSRSNTEDFPLRLESAAPSQTSVYFCASSY

SEQ ID NO: 21 - protein, 96 aa, unidentified
GAGVSQSPSNKVTEKGKDVELRCDPISGHTALYWYRQSLGQGLEFLIYFQGNSAPDKSGLPSDRFSAERTGGSVSTLTIQRTQQEDSAVYLCASSL

SEQ ID NO: 22 - protein, 96 aa, unidentified
GAGVSQTPSNKVTEKGKYVELRCDPISGHTALYWYRQSLGQGPEFLIYFQGTGAADDSGLPNDRFFAVRPEGSVSTLKIQRTERGDSAVYLCASSL

SEQ ID NO: 23 - protein, 96 aa, unidentified
GAGVSQSPRYKVAKRGRDVALRCDSISGHVTLYWYRQTLGQGSEVLTYSQSDAQRDKSGRPSGRFSAERPERSVSTLKIQRTEQGDSAVYLCASSL

SEQ ID NO: 24 - protein, 96 aa, unidentified
GAGVSQSPRYKVTKRGQDVALRCDPISGHVSLYWYRQALGQGPEFLTYFNYEAQQDKSGLPNDRFSAERPEGSISTLTIQRTEQRDSAMYRCASSL

SEQ ID NO: 25 - protein, 96 aa, unidentified
GAGVSQSPRYKVTKRGQDVTLRCDPISSHATLYWYQQALGQGPEFLTYFNYEAQPDKSGLPSDRFSAERPEGSISTLTIQRTEQRDSAMYRCASSL

SEQ ID NO: 26 - protein, 96 aa, unidentified
GAGVSQSPRYKVAKRGQDVALRCDPISGHVSLFWYQQALGQGPEFLTYFQNEAQLDKSGLPSDRFFAERPEGSVSTLKIQRTQQEDSAVYLCASSL

SEQ ID NO: 27 - protein, 96 aa, unidentified
DTGVSQNPRHKITKRGQNVTFRCDPISEHNRLYWYRQTLGQGPEFLTYFQNEAQLEKSRLLSDRFSAERPKGSFSTLEIQRTEQGDSAMYLCASSL

SEQ ID NO: 28 - protein, 95 aa, unidentified
DAEITQSPRHKITETGRQVTLACHQTWNHNNMFWYRQDLGHGLRLIHYSYGVQDTNKGEVSDGYSVSRSNTEDLPLTLESAASSQTSVYFCASSE

SEQ ID NO: 29 - protein, 95 aa, unidentified
DAGITQSPRYKITETGRQVTLMCHQTWSHSYMFWYRQDLGHGLRLIYYSAAADITDKGEVPDGYVVSRSKTENFPLTLESATRSQTSVYFCASSE

SEQ ID NO: 30 - protein, 95 aa, unidentified
DAGITQSPRHKVTETGTPVTLRCHQTENHRYMYWYRQDPGHGLRLIHYSYGVKDTDKGEVSDGYSVSRSKTEDFLLTLESATSSQTSVYFCAISE

SEQ ID NO: 31 - protein, 96 aa, unidentified
EAEVAQSPRYKITEKSQAVAFWCDPISGHATLYWYRQILGQGPELLVQFQDESVVDDSQLPKDRFSAERLKGVDSTLKIQPAELGDSAMYLCASSL

SEQ ID NO: 32 - protein, 96 aa, unidentified
EAGVAQSPRYKIIEKRQSVAFWCNPISGHATLYWYQQILGQGPKLLIQFQNNGVVDDSQLPKDRFSAERLKGVDSTLKIQPAKLEDSAVYLCASSL

SEQ ID NO: 33 - protein, 96 aa, unidentified
EAGVVQSPRYKIIEKKQPVAFWCNPISGHNTLYWYLQNLGQGPELLIRYENEEAVDDSQLPKDRFSAERLKGVDSTLKIQPAELGDSAVYLCASSL

SEQ ID NO: 34 - protein, 96 aa, unidentified
EAGVTQFPSHSVIEKGQTVTLRCDPISGHDNLYWYRRVMGKEIKFLLHFVKESKQDESGMPNNRFLAERTGGTYSTLKVQPAELEDSGVYFCASSQ

SEQ ID NO: 35 - protein, 95 aa, unidentified
DAMVIQNPRYQVTQFGKPVTLSCSQTLNHNVMYWYQQKSSQAPKLLFHYYDKDFNNEADTPDNFQSRRPNTSFCFLDIRSPGLGDTAMYLCATSR

SEQ ID NO: 36 - protein, 95 aa, unidentified
DGGITQSPKYLFRKEGQNVTLSCEQNLNHDAMYWYRQDPGQGLRLIYYSQIVNDFQKGDIAEGYSVSREKKESFPLTVTSAQKNPTAFYLCASSI

SEQ ID NO: 37 - protein, 96 aa, unidentified
DADVTQTPRNRITKTGKRIMLECSQTKGHDRMYWYRQDPGLGLRLIYYSFDVKDINKGEISDGYSVSRQAQAKFSLSLESAIPNQTALYFCATSDL

SEQ ID NO: 38 - protein, 95 aa, unidentified
EADIYQTPRYLVIGTGKKITLECSQTMGHDKMYWYQQDPGMELHLIHYSYGVNSTEKGDLSSESTVSRIRTEHFPLTLESARPSHTSQYLCASSE

SEQ ID NO: 39 - protein, 95 aa, unidentified
EAQVTQNPRYLITVTGKKLTVTCSQNMNHEYMSWYRQDPGLGLRQIYYSMNVEVTDKGDVPEGYKVSRKEKRNFPLILESPSPNQTSLYFCASSL

SEQ ID NO: 40 - protein, 95 aa, unidentified
DVKVTQSSRYLVKRTGEKVFLECVQDMDHENMFWYRQDPGLGLRLIYFSYDVKMKEKGDIPEGYSVSREKKERFSLILESASTNQTSMYLCASSL

SEQ ID NO: 41 - protein, 96 aa, unidentified
SAVISQKPSRDICQRGTSLTIQCQVDSQVTMMFWYRQQPGQSLTLIATANQGSEATYESGFVIDKFPISRPNLTFSTLTVSNMSPEDSSIYLCSVE

SEQ ID NO: 42 - protein, 94 aa, unidentified
SQTIHQWPATLVQPVGSPLSLECTVEGTSNPNLYWYRQAAGRGLQLLFYSVGIGQISSEVPQNLSASRPQDRQFILSSKKLLLSDSGFYLCAWS

SEQ ID NO: 43 - protein, 101 aa, unidentified
AGHLEQPQISSTKTLSKTARLECVVSGITISATSVYWYRERPGEVIQFLVSISYDGTVRKESGIPSGKFEVDRIPETSTSTLTIHNVEKQDIATYYCALWE

SEQ ID NO: 44 - DNA, 406 bp, unidentified
  source 1..406: mol_type = unassigned DNA
agatctccgccgccaccatggcatgccctggcttcctgtgggcacttgtgatctccacctgtcttgaattttccatggcccgtacgggcgccggacacctggaacagccccagatcagcagcaccaagaccctgagcaagtgagacgtttcctgatggactgacgtctcacgtggtgtccggcatcaccatcagcgccacctccgtgtactggtacagagagagacccggcgaggtcatccagttcctggtgtccatcagctacgacggcaccgtgcggaaagagagcggcatccccagcggcaagttcgaggtggactacatccccgagaccagcacctccaccctgaccatccacaacgtggagaagcaggacatcgccacctactactgcgccctgtgggagg

SEQ ID NO: 45 - DNA, 30 bp, unidentified
  source 1..30: mol_type = unassigned DNA
agagacgtttcctgatggactgacgtctca

SEQ ID NO: 46 - DNA, 30 bp, unidentified
  source 1..30: mol_type = unassigned DNA
tgagacgtttcctgatggactgacgtctca

SEQ ID NO: 47 - protein, 21 aa, unidentified
AGHLEQPQISSTKTLSKXAXL

SEQ ID NO: 48 - protein, 47 aa, unidentified
CVVSGITISATSVYWYRERPGEVIQFLVSISYDGTVRKESGIPSGKF

SEQ ID NO: 49 - protein, 30 aa, unidentified
VXIPETSTSXLXIXNVEKQDIATYYCALWE

SEQ ID NO: 50 - protein, 95 aa, unidentified
EPEVTQTPSHQVTQMGQEVILRCVPISNHLYFYWYRQILGQKVEFLVSFYNNEISEKSEIFDDQFSVEPDGSNFTLKIRSTKLEDSAMYFCASSE

SEQ ID NO: 51 - protein, 10 aa, unidentified
KFEVDRIPET

SEQ ID NO: 52 - protein, 96 aa, unidentified
AIELVPEHQTVPVSIGVPATLRCSMKGEAIGNYYINWYRKTQGNTMTFIYREKDIYGPGFKDNFQGDIDIAKNLAVLKILAPSERDEGSYYCACDT

SEQ ID NO: 53 - protein, 96 aa, unidentified
AIELVPEHQTVPVSIGVPATLRCSMKGEAIVNYYINWYRKTQGNTMTFIYREKDIYGPGFKDNFQGDIDIAKNLAVLKILAPSERDEGSYYCACDT

SEQ ID NO: 54 - protein, 96 aa, unidentified
AIELVPEHQTVPVSIGVPATLRCSMKGEAIMNYYINWYRKTQGNTMTFIYREKDIYGPGFKDNFQGDIDIAKNLAVLKILAPSERDEGSYYCACDT

SEQ ID NO: 55 - protein, 96 aa, unidentified
AIELVPEHQTVPVSIGVPATLRCSMKGEAIGNYYINWYRKTQGNTMTFIYRESDIYGPGFKDNFQGDIDIAKNLAVLKILAPSERDEGSYYCACDT

SEQ ID NO: 56 - protein, 96 aa, unidentified
AIELVPEHQTVPVSIGVPATLRCSMKGEAIGNYYINWYRKTQGNTMTFIYREADIYGPGFKDNFQGDIDIAKNLAVLKILAPSERDEGSYYCACDT

SEQ ID NO: 57 - protein, 96 aa, unidentified; identical to SEQ ID NO: 52

SEQ ID NO: 58 - protein, 96 aa, unidentified
AIELVPEHQTVPVSIGVPATLRCSMKGEAIGNYYINWYRKTQGNTITFIYREKDIYGPGFKDNFQGDIDIAKNLAVLKILAPSERDEGSYYCACDT

SEQ ID NO: 59 - protein, 96 aa, unidentified
AIELVPEHQTVTVSMGVPATLRCSMKGEAIGNYYINWYRKTQGNTMTFIYREKDIYGPGFKDNFQGDIDIAKNLAVLKILAPSERDEGSYYCACDT

SEQ ID NO: 60 - protein, 96 aa, unidentified
AIELVPEHQTVPVSIGVPATLRCSMKGEAIGNYYINWYRKTQGNTITFIYREKDIYGPGFKDNFQGDIDIAKNLAVLRILAPSERDEGSYYCACDT

SEQ ID NO: 61 - protein, 96 aa, unidentified
AIELVPEHQTVTVSMGVPATLSCSMKGEAIGNYYINWYRKTQGNTMTFIYREKDIYGPGFKDNFQGDIDIAKNLAVLKILAPSERDEGSYYCACDT

SEQ ID NO: 62 - protein, 96 aa, unidentified
AIELVPEHQTVTVSMGVPATLRCSMKGEAISNYYINWYRKTQDNTMTFIYREKDIYGPGFKDNFQGDIDIAKNLAVLKILAPSERDEGSYYCACDT

SEQ ID NO: 63 - protein, 95 aa, unidentified
AIELVPEHQTVTVSMGVPATLRCSMKGGAISNYYINWYRKTQGNTMTFIYREKGIYGPGFKDNFQGDIDTAKDLAVLKILAPSERDEGSYYCACD

SEQ ID NO: 64 - protein, 95 aa, unidentified
AVELVPEHQTVIVSVGDPATLKCSMKGEAISNYYINWYRKTLGNTMTFIYREKGIYGPGFKDNFQGDIDTEENQAVLKILAPSERDEGSYYCASD

SEQ ID NO: 65 - protein, 95 aa, unidentified
AVELVPEHQTVIVSVGDPATLKCSMKGEAISNYYINWYRKTQGNTMTFIYREKGIYGPGFKDNFQGDIDTEENQAVLKILAPSERDEGSYYCASD

SEQ ID NO: 66 - protein, 93 aa, unidentified
QLVPEHQTVTVSLGDPATLRCSMKGEAITNYYINWYRKTQGNTMTFIYREKDIYGPGFKDGFQGEIDVEKDQAVLEILAASERDEGSYYCASD

SEQ ID NO: 67 - protein, 95 aa, unidentified
ATELVPEHQTVTVSLGEPATLRCSMKGEAITNYYINWYRKTQGNTMTFIYREKDIYGPGFKDGFQGEIDTEKDQAVLEILAASQRDEGSYYCAGD

SEQ ID NO: 68 - protein, 95 aa, unidentified
AIELVPEHQTVTVSLGDPATLRCSMKGEAITNYYISWYRKTQGNTMTFIYREKNIYGPGFKDGFQGEIDPEKDQAVLEILAASERDEGSYYCASD

SEQ ID NO: 69 - DNA, 791 bp, unidentified
  source 1..791: mol_type = unassigned DNA
ggtaccccgccgccaccatggcatgccctggcttcctgtgggcacttgtgatctccacctgtcttgaattttccatggccagcgctatcgagctggtgcccgagcaccagaccgtgcccgtgagcatcggcgtgcccgccaccctgcggtgcagcatgaagggcgaggccatcggcaactactacatcaactggtacagaaagacccagggcaacaccatgaccttcatctaccgggagaaggacatctacggccctggcttcaaggacaacttccagggcgacatctgagacgtttcctaataaaatgacgtctcactggccgtgctgaagatcctggcccccagcgagagggacgagggcagctactactgcgcctgcgacactgccgggggttcttgggacactcgccaaatgttctttggcaccggtataaaactctttgtggagcccagaagccagccccacaccaagcccagcgtgttcgtgatgaagaacggcaccaacgtggcctgcctggtgaaagagttctaccccaaggacatccggatcaacctggtgtccagcaagaagatcaccgagttcgaccccgccatcgtgatcagccccagcggcaagtacaacgccgtgaagctgggcaagtacgaggacagcaacagcgtgacctgcagcgtgcagcacgacaacaagaccgtgcacagcaccgacttcgaggtgaaaaccgactccaccgaccacgtgaagcccaaagagaccgagaacaccaagcagcccagcaagagctgaggatcc